(19) United States
(12) Patent Application Publication
(10) Pub. No.: US 2017/0306406 A1
(43) Pub. Date: Oct. 26, 2017

(54) DNA METHYLATION MARKERS FOR NEURODEVELOPMENTAL SYNDROMES

(71) Applicant: THE HOSPITAL FOR SICK CHILDREN, Toronto (CA)

(72) Inventors: Rosanna WEKSBERG, Toronto (CA); Sanaa CHOUFANI, Maple (CA); Daria GRAFODATSKAYA, Mississauga (CA); Darci BUTCHER, Toronto (CA)

(21) Appl. No.: 15/520,570

(22) PCT Filed: Oct. 21, 2015
(86) PCT No.: PCT/CA2015/051059

§ 371 Date: Apr. 20, 2017

Related U.S. Application Data
(60) Provisional application No. 62/115,922, filed on Feb. 13, 2015.
(60) Provisional application No. 62/067,073, filed on Oct. 22, 2014.

(51) Int. Cl. C12Q 1/68

(52) U.S. Cl. CPC C12Q 1/6883; C12Q 2600/118; C12Q 2600/154

(57) ABSTRACT

The present disclosure provides epigenetic signatures, comprising genomic CpG dinucleotide sequences, genes, and/or genomic regions, which are differentially methylated in individuals with CHARGE syndrome relative to non-CHARGE syndrome controls, and their use in methods and kits for detecting and/or screening for CHARGE syndrome, or the likelihood of CHARGE syndrome. The present disclosure also provides epigenetic signatures, comprising genomic CpG dinucleotide sequences, genes, and/or genomic regions, which are differentially methylated in individuals with Kabuki syndrome relative to non-Kabuki syndrome controls, and their use in methods and kits for detecting and/or screening for Kabuki syndrome, or the likelihood of Kabuki syndrome.

### RELATED APPLICATION

This application claims the benefit of priority to U.S. Provisional Applications Nos. 62/067,073 filed Oct. 22, 2014 and 62/115,922 filed Feb. 13, 2015, respectively. The contents of which are incorporated herein by reference in their entirety.

### FIELD

The disclosure relates to methods and kits for detecting and/or screening for CHARGE syndrome (CS), or an increased likelihood of CS, in a human subject. The disclosure further relates to methods and kits for detecting and/or screening for Kabuki syndrome (KS), or an increased likelihood of KS, in a human subject.

### INTRODUCTION

Epigenetics, which refers to changes in gene expression that occur without a change in DNA sequence¹, is a vital genome-wide regulatory system, the primary function of which is to modulate gene expression. Epigenetic regulation determines where and when genes are expressed via a number of mechanisms including DNA methylation, histone modifications and

ATP-dependent chromatin remodeling. According to the Disease Annotated Chromatin Epigenetics Resource (DAnCER)² 633 human genes encode proteins that have experimentally confirmed involvement in regulating epigenetic modifications and chromatin remodeling. An additional ˜1,600 genes have been predicted, using bioinformatics tools, to be involved in epigenetic regulation².

To date, mutations and deletions or insertions in just over 30 of these genes with known functions in regulating the epigenome have been identified as being causative in syndromic and non-syndromic intellectual disability (S-ID and NS-ID)³⁻¹⁴. One of these genes is chromodomain helicase DNA-binding protein 7 (CHD7). A member of a family of chromatin remodeling proteins, CHD7 has been shown to be important in early embryonic development. CHD7 is expressed in human embryonic stem (hES) cells and that expression is increased, and required, for hESs to form multipotent migratory neural crest like cells (hNCLC)¹⁵. Neural crest cells (NCC) contribute to a number of tissues in the developing embryo¹⁶. In animal models, both mouse and Xenopus laevis, knockdown of CHD7 disrupts the migration of NCC¹⁷⁻¹⁹. Hemizygosity of CHD7 results in the aberrant development of craniofacial structures, heart and other organ abnormalities^{19,20}.

CHARGE syndrome can be clinically characterized by the coloboma of the eye, heart defects, choanal atresia, retardation of growth and development, genital hypoplasia, and ear/deafness/vestibular/olfactory/other cranial nerve disorders²¹. Its incidence is 1 in 8 500 to 10 000 live births^{22,23.} CHARGE syndrome patients face a wide variety of life-threatening conditions, with high mortality rates in the first year of life, including cardiac abnormalities, feeding and/or breathing difficulties²³. The majority of CHARGE syndrome (OMIM #214800) cases (˜60% to 80%) are due to haploinsufficiency of CHD7, due to de novo nonsense, deletion, or missense mutations²⁴. More than 500 pathogenic mutations in CHD7 have been identified, many of which are unique to the patient^{25,26}.

In human cell lines using chromatin immunoprecipitation (ChIP) CHD7 has been shown to bind to chromatin regions that are active as demonstrated by histone H3 lysine 4 methylation (H3K4) and DNAse1 hypersensitivity of these binding sites²⁷′²⁸. CHD7 binding sites in hES are localized to enhancers and promoters determined by overlapping features, including p300 binding, H3K4 mono-, di- and tri methylation²⁸. It has been previously determined that loss of function mutations in KDMSC (OMIM#314690), an H3K4 demethylase, causes alterations in DNA methylation demonstrating cross talk between DNA methylation and chromatin modification²⁹.

Phenotypic overlap between CHARGE syndrome and another neurodevelopmental syndrome, Kabuki syndrome, can sometimes lead to the consideration of CHARGE syndrome in individuals with Kabuki syndrome. Indeed, CHARGE syndrome and Kabuki syndrome are both undergrowth syndromes. Undergrowth refers to growth deficiency compared to the norms of the population and usually affects height and weight. Growth of the head may be normal or deficient Kabuki syndrome (OMIM #147920) is a disorder with a prevalence of 1 in 32,000 births, characterized by distinct facial characteristics (inverted lower eyelids, long palpebral fissures, large dysplastic ears, arched eyebrows, short nasal septum, cleft palate and abnormal teeth), various degrees of intellectual disability and other congenital malformations (cardiac, renal and skeletal)³⁴.

In 2010, mutations in the KMT2D (also known as MLL2) gene were identified as the cause of the majority of Kabuki syndrome cases³³. KMT2D, located on chromosome 12, belongs to the trithorax group of histone modifying proteins. It contains several domains suited for its function, including a PHD domain for histone binding, a FYRN domain found in chromatin associating proteins and a SET domain found in many methyltransferases. The Drosophila homolog of the KMT2D gene, trithorax-related (trr), has been demonstrated to trimethylate histone H3 lysine 4. This histone mark is commonly found in active or poised chromatin regions. Normal epigenetic marks, including DNA methylation (DNAm) and histone modifications, are established and maintained by genes that can be defined as “epigenes”. Mutations in epigenes result in a number of neurodevelopmental disorders, including Kabuki syndrome. Histone modifications and DNA methylation have been shown to interact through crosstalk between proteins and protein complexes which regulate chromatin structure. Specific histone marks are commonly associated with DNAm and methylation of specific CpG sites accompanying specific histone modifications. The present inventors have previously determined that loss of function mutations in KDMSC (OMIM#314690), an H3K4 demethylase, causes alterations in DNA methylation demonstrating cross talk between DNA methylation and chromatin modification³⁵.

There is a need for robust and cost-effective tests capable of identifying neurodevelopmental syndromes such as CHARGE syndrome cases and Kabuki syndrome cases, with high specificity and sensitivity. These tests may be used to identify CHARGE syndrome and Kabuki syndrome in individuals carrying variants of unknown significance.

### SUMMARY

The present disclosure provides DNA methylation markers which are capable of differentiating CHARGE syndrome (CS) cases carrying a pathogenic CHD7 mutation from non-CHARGE syndrome (non-CS) controls, including distinguishing CHARGE syndrome cases from individuals carrying a benign CHD7 variant (benign variant as referred to herein means a variant in CHD7 gene that does not alter protein function). The DNA methylation markers and the methods of their use described herein may provide useful alternative or supplementary diagnostics to currently available methods of detecting and/or screening for CS, or likelihood of CS.

In an aspect, there is provided a method of detecting and/or screening for CHARGE syndrome (CS), or an increased likelihood of CS, in a human subject, comprising determining a sample DNA methylation profile from a sample of DNA from said subject, said sample profile comprising the methylation level of at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i).

The method further comprises determining the level of similarity of said sample profile to one or more control profiles, wherein (i) a high level of similarity of the sample profile to a CS specific control profile; (ii) a low level of similarity to a non-CS control profile; and/or (iii) a higher level of similarity to a CS specific control profile than to a non-CS control profile indicates the presence of, or an increased likelihood of, CS.

In an embodiment, the CpG loci comprise (i) CpG loci from Tables 2 and/or 16 having an absolute CS delta-beta value ≧0.10, optionally ≧0.11, ≧0.12, ≧0.13, ≧0.15, ≧0.18, ≧0.20 or ≧0.22; and/or (ii) associated

CpG loci residing within 300 nucleotides, optionally within 150 nucleotides, of the CpG loci of (i).

In another aspect, there is provided a method of detecting and/or screening for CHARGE syndrome (CS), or an increased likelihood of CS, in a human subject, comprising:

determining a sample methylation profile from a sample of DNA from said subject, said sample profile comprising the methylation level of CpG loci, wherein the CpG loci are the loci from Tables 2 and/or 16 having an absolute CS delta-beta value ≧0.1; and

determining the level of similarity of said sample profile to one or more control profiles, wherein (i) a high level of similarity of the sample profile to an CS specific control profile; (ii) a low level of similarity to a non-CS control profile; and/or (iii) a higher level of similarity to a CS specific control profile than to a non-CS control profile indicates the presence of, or an increased likelihood of, CS.

In an embodiment, the CpG loci comprise CpG loci from Tables 2 and/or 16 having an absolute CS delta-beta value ≧0.10, optionally ≧0.11, ≧0.12, ≧0.13, ≧0.15, ≧0.18, ≧0.20 or ≧0.22.

In another embodiment, determining the sample methylation profile comprises the steps:
a) providing the sample comprising genomic DNA from the subject;
b) optionally, isolating DNA from the sample;
c) optionally, treating DNA from the sample with sodium bisulfite for a time and under conditions sufficient to convert non-methylated cytosines to uracils;
d) optionally, amplifying the DNA; and
e) determining the methylation level at the CpG loci by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing (RRBS), and/or enrichment-based methods such as MeDIP-seq, MBD-seq, or MRE-seq.

In another embodiment, the correlation coefficient is a linear correlation coefficient, optionally a Pearson correlation coefficient or a Spearman correlation coefficient.

In another embodiment, a higher level of similarity to the CS specific control profile than to the non-CS control profile is indicated by a higher correlation value computed between the sample profile and the CS specific control profile than an equivalent correlation value computed between the sample profile and the non-CS control profile, optionally wherein the correlation value is a correlation coefficient.

In yet another embodiment, a high level of similarity to the control profile is indicated by a Pearson correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In an embodiment, the methylation level is measured as a β-value.

In another embodiment, a Charge Syndrome Score (Charge score) is calculated according to following formula:

Charge score(B)=r (B, Charge profile)−r (B, non-Charge profile)

where r is a Pearson correlation coefficient, and B is a vector of DNA methylation levels across the selected methylation loci in the sample.

In another embodiment, determining the sample methylation profile comprises contacting the DNA with at least one agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG, and (b) an oligonucleotide comprising nucleotide sequence complementary to or identical to a nucleotide sequence of genomic DNA comprising the same selected CpG locus of (a), in which the cytosine residue of the CpG locus is replaced with a thymine residue.

In yet another embodiment, the contacting is under hybridizing conditions.

In an embodiment, the methylation levels of the selected loci of at least one control profile is derived from one or more samples, optionally from historical methylation data for a patient or pool of patients.

In another embodiment, the non-CS control profile comprises methylation levels for the selected CpG loci listed in Tables 2 and/or 16. In yet another embodiment, the CS specific control profile comprises DNA methylation levels for the selected CpG loci listed in Tables 2 and/or 16. In an embodiment, the methylation levels of associated CpG loci not listed in Tables 2 and/or 16 is assumed to be equivalent to the methylation level of a CpG loci listed in Tables 2 and/or 16 with which the CpG loci is associated.

In an embodiment, the sample is derived from blood, fibroblast tissue, buccal tissue, lymphoblastoid cell line, saliva or a prenatal sample. The prenatal sample is optionally a CVS, placenta, circulating fetal DNA and/or amniotic fluid sample. In another embodiment, the sample is derived from a tissue biopsy.

In another embodiment, the human subject is a fetus.

Another aspect provides a method of detecting and/or screening for CHARGE syndrome (CS), or an increased likelihood of CS, in a human subject, comprising determining a sample DNA methylation profile from a sample of DNA from said subject, said sample profile comprising the methylation level of at least 2, optionally at least 3, at least 4, at least 6, at least 8, at least 10, at least 16, at least 20, at least 25, at least 30, at least 35, at least 40, or all the genes from Tables 2 and/or 16.

The method further comprises determining the level of similarity of said sample profile to one or more control profiles, wherein (i) a high level of similarity of the sample profile to a CS specific control profile; (ii) a low level of similarity to a non-CS control profile; and/or (iii) a higher level of similarity to a CS specific control profile than to a non-CS control profile indicates the presence of, or an increased likelihood of, CS.

Another aspect of the disclosure provides a method of assigning a course of management for an individual with CHARGE syndrome (CS), or an increased likelihood of CS, comprising:
a) identifying an individual with CS or an increased likelihood of CS, according to the methods described herein; and
b) assigning a course of management for CS and/or symptoms of a

CS, comprising i) testing for at least one medical condition associated with CS and ii) applying an appropriate medical intervention based on the results of the testing.

In one embodiment, the medical condition is selected from ophthalmic colobomas, cardiovascular anomalies, hearing loss, airway conditions such as choanal atresia/stenosis or tracheoesophageal fistula, feeding issues, retinal detachment, growth delay, delayed puberty, renal anomalies, developmental difficulties, behavioural problems, dual sensory loss and/or neuropsychological issues such as attention deficit hyperactivity disorder or autism.

Another aspect of the disclosure provides a kit for detecting and/or screening for CHARGE syndrome, or an increased likelihood of CS, in a sample, comprising:
a) at least one detection agent for determining the methylation level of:
   i) at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i); and/or
   ii) at least 2, optionally at least 3, at least 4, at least 6, at least 8, at least 10, at least 16, at least 20, at least 25, at least 30, at least 35, at least 40, or all the genes from Tables 2 and/or 16; and
b) instructions for use.

In an embodiment, the kit further comprises bisulfite conversion reagents, methylation-dependent restriction enzymes, methylation-sensitive restriction enzymes, PCR reagents, probes and/or primers.

In an embodiment, the kit further comprises a computer-readable medium that causes a computer to compare methylation levels from a sample at the selected CpG loci to one or more control profiles and computes a correlation value between the sample and control profile. In an embodiment, the computer readable medium obtains the control profile from historical methylation data for a patient or pool of patients known to have, or not have, CHARGE syndrome. In some embodiments, the computer readable medium causes a computer to update the control profile based on the testing results from the testing of a new patient.

The present disclosure also provides DNA methylation markers which are capable of differentiating Kabuki syndrome (KS) cases carrying a pathogenic KMT2D mutation from non-Kabuki syndrome (non-KS) controls, including distinguishing Kabuki syndrome cases from individuals carrying a benign KMT2D variant (benign variant as referred to herein means a variant in KMT2D gene that does not alter protein function). The DNA methylation markers and the methods of their use described herein may provide useful alternative or supplementary diagnostics to currently available methods of detecting and/or screening for KS, or likelihood of KS.

Accordingly, an aspect of the disclosure provides a method of detecting and/or screening for Kabuki syndrome (KS), or an increased likelihood of KS, in a human subject, comprising:
determining a sample methylation profile from a sample comprising DNA from said subject, said sample profile comprising the methylation level of at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and
determining the level of similarity of said sample profile to one or more control profiles, wherein (i) a high level of similarity of the sample profile to a KS specific control profile; (ii) a low level of similarity to a non-KS control profile; and/or (iii) a higher level of similarity to a KS specific control profile than to a non-KS control profile indicates the presence of, or an increased likelihood of, KS.

In one embodiment, the selected CpG loci comprise CpG loci from Tables 9 and/or 17 having an absolute KS delta-beta value ≧0.15, optionally ≧0.16, ≧0.18, ≧0.20, ≧0.22, ≧0.24 or ≧0.25; and/or (ii) associated CpG loci residing within 300 nucleotides, optionally within 150 nucleotides, of the CpG loci of (i).

Another aspect of the disclosure provides a method of detecting and/or screening for Kabuki syndrome (KS), or an increased likelihood of KS, in a human subject, comprising:
determining a sample methylation profile from a sample comprising DNA from said subject, said sample profile comprising the methylation level of CpG loci, wherein the CpG loci are the loci from Tables 9 and/or 17; and
determining the level of similarity of said sample profile to one or more control profiles, wherein (i) a high level of similarity of the sample profile to a KS specific control profile; (ii) a low level of similarity to a non-KS control profile; and/or (iii) a higher level of similarity to a KS specific control profile than to a non-KS control profile indicates the presence of, or an increased likelihood of, KS.

In one embodiment, the selected CpG loci comprise the CpG loci from Tables 9 and/or 17 having an absolute KS delta-beta value ≧0.16.

In one embodiment, the selected CpG loci comprise the CpG loci from Tables 9 and/or 17 having an absolute KS delta-beta value ≧0.18.

In another embodiment, the selected CpG loci comprise the CpG loci from Tables 9 and/or 17 having an absolute KS delta-beta value ≧0.20.

In another embodiment, the selected CpG loci comprise the CpG loci from Tables 9 and/or 17 having an absolute KS delta-beta value ≧0.22.

In another embodiment, the selected CpG loci comprise the

CpG loci from Tables 9 and/or 17 having an absolute KS delta-beta value ≧0.24.

In another embodiment, the selected CpG loci comprise the CpG loci from Tables 9 and/or 17 having an absolute KS delta-beta value ≧0.25.

In another embodiment, determining the sample methylation profile comprises the steps:
a) providing the sample comprising genomic DNA from the subject;
b) optionally, isolating DNA from the sample;
c) optionally, treating DNA from the sample with bisulfite for a time and under conditions sufficient to convert non-methylated cytosines to uracils;
d) optionally, amplifying the DNA; and
e) determining the methylation level at the CpG loci by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing(RRBS), and/or enrichment-based methods such as MeDIP-seq, MBD-seq, or MRE-seq.

In another embodiment, a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In another embodiment, a higher level of similarity to the KS specific profile than to the non-KS control profile is indicated by a higher correlation value computed between the sample profile and the KS specific profile than an equivalent correlation value computed between the sample profile and the non-KS control profile, optionally wherein the correlation value is a correlation coefficient.

In another embodiment, the correlation coefficient is a linear correlation coefficient, optionally a Pearson correlation coefficient.

In another embodiment, methylation level is measured as a β-value. Optionally, hypermethylation is indicated by the gene having a significantly higher methylation beta value in the KS specific control profile compared to the non-KS control profile and hypomethylation is indicated by the gene having a significantly lower methylation beta value in the KS specific control profile compared to the non-KS control profile.

In another embodiment, determining the profile of methylated DNA from the subject comprises contacting the DNA with at least one agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG loci, and (b) an oligonucleotide comprising nucleotide sequence complementary to or identical to a nucleotide sequence of genomic DNA comprising the same selected CpG loci of (a), in which the cytosine residue of the CpG loci is replaced with a thymine residue.

In another embodiment, the contacting is under hybridizing conditions.

In another embodiment, the methylation levels of the selected loci of at least one control profile is derived from one or more samples, optionally from historical methylation data for a patient or pool of patients.

In another embodiment, the non-KS control profile comprises methylation levels for the selected CpG loci listed in Tables 9 and/or 17.

In another embodiment, the KS specific control profile comprises methylation levels for the selected CpG loci listed in Tables 9 and/or 17.

In another embodiment, the methylation level of a selected CpG locus not listed in Tables 9 and/or 17 is assumed to be equivalent to the methylation level of a CpG locus listed in Tables 9 and/or 17 with which the selected DNA CpG locus is associated.

In another embodiment, the sample is derived from blood, fibroblast tissue, buccal tissue, lymphoblastoid cell line, saliva or a prenatal sample, optionally a CVS, placenta, circulating fetal DNA and/or amniotic fluid sample.

In another embodiment, the human subject is a fetus.

The present disclosure also provides a method of detecting and/or screening for Kabuki syndrome (KS), or an increased likelihood of KS, in a human subject, comprising:
determining a sample methylation profile from a sample comprising DNA from said subject, said sample profile comprising the methylation level of at least 3, optionally at least 4, at least 6, at least 8, at least 10, at least 15, at least 20, at least 25, at least 50, at least 75, at least 100, at least 125, or all the genes from Tables 9 and/or 17; and
determining the level of similarity of said sample profile to one or more control profiles, wherein (i) a high level of similarity of the sample profile to an KS specific control profile; (ii) a low level of similarity to a non-KS control profile; and/or (iii) a higher level of similarity to a KS specific control profile than to a non-KS control profile indicates the presence of, or an increased likelihood of, KS.

In one embodiment, the genes are FAM65B, HOXC4 and MYO1F.

In one embodiment, determining the methylation levels of the selected genes comprises the steps:
a) providing the sample comprising genomic DNA from the subject;
b) optionally, isolating DNA from the sample;
c) optionally, treating DNA from the sample with bisulfite for a time and under conditions sufficient to convert non-methylated cytosines to uracils;
d) optionally, amplifying the DNA; and
e) determining the methylation status at the selected genes by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing (RRBS), and/or enrichment-based methods such as MeDIP-seq, MBD-seq, or MRE-seq.

In one embodiment, the methylation level is measured as a β-value.

In another embodiment, hypermethylation is indicated by the gene having a significantly higher methylation beta value in the KS specific control profile compared to the non-KS control profile and hypomethylation is indicated by the gene having a significantly lower methylation beta value in the KS specific control profile compared to the non-KS control profile.

In another embodiment, the sample is derived from blood, fibroblast tissue, buccal tissue, lymphoblastoid cell line, saliva or a prenatal sample, optionally a CVS, placenta, circulating fetal DNA and/or amniotic fluid sample.

In another embodiment, the human subject is a fetus.

The present disclosure also provides a method of determining a course of management for an individual with Kabuki syndrome (KS), or an increased likelihood of KS, comprising:
a) identifying an individual with KS or an increased likelihood of KS, according to the methods described herein; and
b) assigning a course of management for KS and/or symptoms of a KS, comprising i) testing for at least one medical condition associated with KS and ii) applying an appropriate medical intervention based on the results of the testing.

In one embodiment, the medical condition is selected from ophthalmic abnormalities, cardiovascular anomalies, hearing loss, kidney abnormalities, skeletal anomalies, dental abnormalities, feeding difficulties, endocrine problems, infection, autoimmune disorders, seizures and developmental disorders.

The present disclosure further provides a kit for detecting and/or screening for Kabuki syndrome, or an increased likelihood of KS, in a sample, comprising:
at least one detection agent for determining the methylation level of:

at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and/or

at least 3, optionally at least 4, at least 6, at least 8, at least 10, at least 15, at least 20, at least 25, at least 50, at least 75, at least 100, at least 125, or all the genes from Tables 9 and/or 17; and instructions for use.

In one embodiment, the kit further comprises bisulfite conversion reagents, methylation-dependent restriction enzymes, methylation-sensitive restriction enzymes, PCR reagents, probes and/or primers.

In another embodiment, the kit further comprises a computer-readable medium that causes a computer to compare methylation levels from a sample at the selected CpG loci to one or more control profiles and compute a correlation value between the sample and control profile.

### DRAWINGS

Embodiments are described below in relation to the drawings in which:

FIG. 1 is a volcano plot showing the relationship between the average change in blood DNA methylation in the CHD7 nonsense mutation cohort (n=15; Δβ effect size, X-axis), and the statistical significance of such changes (p-value of the Mann-Whitney U test after Benjamini-Hochberg correction for multiple testing, shown in logarithmic scale, Y-axis). Each semi-transparent point represents one of the 432,601 CpG sites. The horizontal line represents the statistical significance level p<0.01.

FIG. 2 shows hierarchical clustering of 15 CHD7 samples (black; bottom row) and 45 control samples (light grey; top row) from blood.

The clustering was generated from the DNA methylation levels across the 146 CpG sites that exhibited significant changes in methylation (p<0.01 and at least 10% DNAm difference) between the two cohorts. Samples with variants in CHD7 (n=14; dark grey, middle row) were added to the clustering to determine if they clustered with the CHD7 pathogenic variants or with the controls.

FIG. 3 shows the classification of various categories of blood DNA methylation samples. Two median-methylation profiles were built over the 146 significant CpGs: one using the 15 CHD7 nonsense pathogenic mutation samples (filled circles), and another using the 45 Control samples (squares). 1056 normal blood DNAm samples derived from GEO (crosses) were examined, 1051 of which were more similar to the Control profile (specificity>99.5%). 14 samples with variants in CHD7 (triangles) were also classified, of which 9 cases showed a higher similarity to the pathogenic nonsense mutation CHD7 cases and the remaining 5 variants of unknown significance (VUS), three were more similar to the controls. Pearson correlation was used as the similarity metric.

FIG. 4 is a volcano plot showing the relationship between the average change in blood DNA methylation in the Kabuki nonsense mutation cohort compared to normal controls (Δβ effect size, X-axis) and the statistical significance of such changes (p-value of the Mann-Whitney U test after Benjamini-Hochberg correction for multiple testing, shown in logarithmic scale, Y-axis). Each semi-transparent point represents one of the 422,139 CpG sites. The horizontal line represents the statistical significance level p=0.05. The vertical lines represent the effect size of 15% change in DNAm. The data cohorts contained 11 Kabuki nonsense samples and 45 normal controls.

FIG. 5 shows hierarchical clustering of 11 Kabuki samples and 45 control samples from blood. The heatmap shows the clustering based on the DNA methylation levels across the 287 CpG sites that exhibited significant changes in methylation (p<0.05 and at least 15% DNAm difference) between the two cohorts. Samples with variants in KMT2D (n=11) were added to the clustering to determine if they clustered with the Kabuki pathogenic samples or with the controls. Clustering was performed based on the Pearson correlation metric with average linkage (correlation scale shown on the right).

FIG. 6 shows classification of various categories of blood DNA methylation samples. Two median-methylation profiles were built over the 287 significant CpGs: one using the 11 Kabuki samples with pathogenic nonsense mutation in KMT2D (circles), and another using the 45 Control samples (squares). 1056 normal blood DNAm samples derived from GEO (crosses) were also examined, all of which were more similar to the Control profile (specificity=100%). 9 samples were classified with variants in KMT2D, of which 1 case showed a higher similarity to the pathogenic nonsense mutation Kabuki cases and the remaining 8 variants were more similar to the controls. The nine samples all had non-synonymous changes (missense mutations) in KMT2D. Two of these patients had clinical features suggestive of possible-Kabuki syndrome and the remaining seven cases were studied to rule out diagnosis of Kabuki syndrome in children with developmental problems. Pearson correlation was used as the similarity metric.

### DESCRIPTION OF VARIOUS EMBODIMENTS

The inventors have conducted genome-wide DNA methylation (DNAm) profiling using blood from individuals with CHARGE syndrome (CS), a disorder involving aberrant CHD7 function. Based on comparison of the DNA methylation profile from CS individuals to those of non-CS controls, the inventors have shown that DNA methylation profiles may be used in a test for early and accurate diagnosis of CHARGE syndrome due to CHD7 pathogenic mutations. 146 CpG loci (Table 2) plus 3 CpG loci (Table 16) were identified as showing a statistically significant (corrected p-value<0.01) difference in methylation levels between CS cases and non-CS controls.

The inventors have also conducted genome-wide DNA methylation (DNAm) profiling using blood from individuals with Kabuki syndrome (KS), a disorder involving aberrant KMT2D function. Based on comparison of the DNA methylation profile from KS individuals to those of non-KS controls, the inventors have shown that DNA methylation profiles may be used in a test for early and accurate diagnosis of Kabuki syndrome due to

KMT2D pathogenic mutations. 287 CpG loci (Table 9) plus 75 CpG loci (Table 17) were identified as showing a statistically significant (corrected p-value≦0.05) difference in methylation levels between KS cases and non-KS controls.

### I. Definitions

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the term “isolated” or “purified” when used in relation to a DNA molecule refers to a DNA molecule that is extracted and separated from one or more contaminants with which it naturally occurs.

As used herein, “methylation” refers specifically to DNA methylation, and more particularly to a modification in which a methyl group or hydroxymethyl group is added to the 5 position of a cytosine residue to form a 5-methyl cytosine (5-mCyt) or 5-hydroxymethylcytosine (5-hmC).

As used herein, “CpG locus” or “methylation locus” refers to an individual CpG dinucleotide sequence in genomic DNA which is capable of being methylated. Individual CpG loci may be identified by reference to an Illumina CpG locus (Illumina ID #) which is defined by a chromosome number, genomic coordinate (referenced to NCBI, hg19), genome build (37), and +/−strand designation to unambiguously define each CpG locus. The genomic information is publically available through the UCSC genome browser at https://cienome.ucsc.edu/.

The term “methylation level” refers to a measure of the amount of methylation at a target site (for example, a CpG locus) within a DNA molecule in a sample. For example, the level of methylation can be measured for one or more CpG dinucleotides, or for a region of DNA. If the methylation level of a target site within a sample is higher than a reference level, the sample is considered to have increased methylation relative to the reference at the target site. Conversely, if the methylation level of a target site within a sample is lower than the reference level, the sample is considered to have a decreased methylation level relative to the reference at the target site. The target site may be an individual CpG locus or a region of DNA comprising multiple CpG loci, for example, a gene promoter. Methylation levels of a target site may be measured by methods known in the art, for example, as a “β value” or “beta value”, which is calculated as:

β value=intensity of the methylated target (M)/(intensity of the unmethylated target (U)+intensity of the methylated target(M)+100)

A β value of zero indicates no methylation and a value of one indicates 100% methylation.

As used herein, the term “methylation status” refers to whether a specified target DNA site is methylated or not methylated. The target site may be an individual CpG locus or a region of DNA comprising multiple CpG loci, for example, a gene promoter. For example, a target site may have a methylation status of “methylated” or “hypermethylated” if the target has significantly higher methylation beta value in a CS (or KS) specific control profile compared to a non-CS (or non-KS) control profile. Conversely, a target site may have a methylation status of “not methylated” or “hypomethylated” if the target has significantly lower methylation beta value in a CS (or KS) specific control profile compared to a non-CS (or non-KS) control profile.

As used herein, the term “delta beta” or “delta β” refers to the difference between the β value of a methylation target in two different samples, for example, the β value of a methylation target in a CS (or KS) specific control profile and the β value of the same methylation target in a non-CS (or non-KS) control profile.

As used herein the term “gene” refers to a genomic DNA sequence that comprises a coding sequence associated with the production of a polypeptide or polynucleotide product (e.g., rRNA, tRNA). The methylation level of a gene as used herein, encompasses the methylation level of sequences which are known or predicted to affect expression of the gene, including the promoter, enhancer, and transcription factor binding sites. As used herein, the term “enhancer” refers to a cis-acting region of DNA that is located up to 1 Mbp (upstream or downstream) of a gene.

As used herein, the term “sample methylation profile” or “sample profile” refers to the methylation levels at one or more target sequences in a subject's genomic DNA. The target sequence may be an individual CpG locus or a region of DNA comprising multiple CpG loci, for example, a gene promoter or CpG island. The methylation profile of a sample tested according the methods disclosed herein is referred to as a sample profile.

In some embodiments, the sample methylation profile is compared to one or more control profiles. The control profile may be a reference value and/or may be derived from one or more samples, optionally from historical methylation data for a patient or pool of patients who are known to have, or not have, CHARGE syndrome or Kabuki syndrome. In such cases, the historical methylation data can be a value that is continually updated as further samples are collected and individuals are identified as CS or not-CS, or KS or not-KS. It will be understood that the control profile represents an average of the methylation levels for selected CpG loci as described herein. Average methylation values may, for example, be the mean values or median values.

For example, a “CS specific control profile” or “CS control profile” may be generated by measuring the methylation levels at specified target sequences in genomic DNA from an individual subject, or population of subjects, who are known to have CS and a CHD7 pathogenic mutation.

Similarly, a “non-CS control profile” may be generated by measuring the methylation levels at specified target sequences in genomic DNA from an individual subject or population of subjects who are known to not have CS.

In another example, a “KS specific control profile” or “KS control profile” may be generated by measuring the methylation levels at specified target sequences in genomic DNA from an individual subject, or population of subjects, who are known to have KS and a KMT2D pathogenic mutation. Similarly, a “non-KS control profile” may be generated by measuring the methylation levels at specified target sequences in genomic DNA from an individual subject or population of subjects who are known to not have KS.

In certain embodiments, the tissue source from which the sample profile and control profile are derived is matched, so that they are both derived from the same or similar tissue.

As used herein, the phrase “detecting and/or screening” for a condition refers to a method or process of determining if a subject has or does not have said condition. Where the condition is a likelihood or risk for a disease or disorder, the phrase “detecting and/or screening” will be understood to refer to a method or process of determining if a subject is at an increased or decreased likelihood for the disease or disorder.

As used herein, the term “sensitivity” refers to the ability of the test to correctly identify those patients with the disease or disorder, such that a 100% sensitivity indicates a test that correctly identifies all patients with the disease or disorder. Sensitivity is calculated as:

Sensitivity=(True Positives)/(True Positives+False Negatives). A high sensitivity as used herein refers to a sensitivity of greater than 50%.

As used herein, the term “specificity” refers to the ability of a test to correctly identify those patients without the disease or disorder, such that a 100% specificity indicates a test that correctly identifies all patients without the disease or disorder. Specificity is calculated as:

Specificity=(True Negatives)/(True Negatives+False Positives). A high specificity as used herein refers to a specificity of greater than 50%.

As used herein, the term “CpG” or “CG” site refers to cytosine and guanosine residues located sequentially (5′->3′) in a polynucleotide DNA sequence. The term “CpG island” refers to a region of genomic DNA characterized by a high frequency of CpG sites, for example, a CpG island may be characterized by CpG dinucleotide content of at least 60% over the length of the island. As used herein the term “CpG island shore” refers to a region of DNA occurring within 2 kbp (upstream or downstream) of a CpG island. As used herein the term “body” (in reference to a gene) refers to the genomic region covering the entire gene from the transcription start site to the end of the transcript. As used herein the term “distance from TSS” refers to the genomic difference in base pairs between specific CpG locus and the nearest transcription start site.

As used herein, a first CpG locus is “associated” with a second CpG locus, if the methylation status at the first locus is reasonably predictive of the methylation status of the second locus and vice versa. CpG loci may be considered “associated”, for example, if they occur within the same CpG island, CpG island shore, gene promoter or gene enhancer region. CpG loci may also be considered “associated” by virtue of their genomic proximity, for example, CpG loci residing within 300 nucleotides, optionally within 150 nucleotides, of each other may be considered associated.

As used herein, the term “treating DNA from the sample with bisulfite” refers to treatment of DNA with a reagent comprising bisulfite, disulfite, hydrogen sulfite or combinations thereof, for a time and under conditions sufficient to convert unmethylated DNA cytosine residues to uracil, thereby facilitating the identification of methylated and unmethylated CpG dinucleotide sequences. Bisulfite modifications to DNA may be detected according to methods known in the art, for example, using sequencing or detection probes which are capable of discerning the presence of a cytosine or uracil residue at the CpG site.

The term “subject” as used herein refers to a human subject and includes, for example, a fetus.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial,” in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

The term “hybridize” refers to the sequence specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0×sodium chloride/sodium citrate (SSC) at about 45° C. for 15 minutes, followed by a wash of 2.0×SSC at 50° C. for 15 minutes may be employed.

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10[Na+])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions. In an embodiment, stringent hybridization conditions are selected. By way of example the following conditions may be employed to achieve stringent hybridization: hybridization at 5×sodium chloride/sodium citrate (SSC)/5×Denhardt's solution/1.0% SDS at Tm −5° C. based on the above equation, followed by a wash of 0.2×SSC/0.1% SDS at 60° C. for 15 minutes. Moderately stringent hybridization conditions include a washing step in 3×SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

The term “oligonucleotide” as used herein refers to a nucleic acid substantially free of cellular material or culture medium when produced by recombinant DNA techniques, or chemical precursors, or other chemicals when chemically synthesized. The term “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand. The term also includes modified or substituted oligomers comprising non-naturally occurring monomers or portions thereof.

As used herein, the term “amplify”, “amplifying” or “amplification” of DNA refers to the process of generating at least one copy of a DNA molecule or portion thereof. Methods of amplification of DNA are well known in the art, including but not limited to polymerase chain reaction (PCR), ligase chain reaction (LCR), self-sustained sequence replication (3SR), nucleic acid sequence based amplification (NASBA), strand displacement amplification (SDA), multiple displacement amplification (MDA) and rolling circle amplification (RCA).

### II. Methods

As set out in Table 2, the instant disclosure identifies 146 distinct CpG loci, each of which show a statistically significant (corrected p-value<0.01) difference in methylation levels between individuals with CS and non-CS controls over the tested population. As set out in Table 16, the instant disclosure identifies an additional 3 CpG loci, each of which show as statistically significant (corrected p-value<0.01) difference in methylation levels between individuals with CS and non-CS controls over the tested population. As described in the Examples, the methylation levels of the disclosed loci, or a subset thereof, may be used in diagnostic testing for CS, with up to 100% sensitivity and specificity. It will be understood that the sensitivity and specificity of the methods described will tend to increase with the number of CpG loci or sites selected for testing (i.e. the size of the signature), to a maximal sensitivity/specificity of 100%. However, signatures utilizing fewer CpG loci, are described herein which retain greater than 50% sensitivity and specificity and are useful for assessing likelihood of CHARGE syndrome.

Further, as set out in Table 9, the instant disclosure identifies 287 distinct CpG loci, each of which show a statistically significant (corrected p-value≦0.05) difference in methylation levels between individuals with KS and non-KS controls over the tested population. Also, as set out in Table 17, the instant disclosure identifies and additional 75 distinct CpG loci, each of which show a statistically significant (corrected p-value≦0.05) difference in methylation levels between individuals with KS and non-KS controls over the tested population. As described in the Examples, the methylation levels of the disclosed loci, or a subset thereof, may be used in diagnostic testing for KS, with up to 100% sensitivity and specificity. It will be understood that the sensitivity and specificity of the methods described will tend to increase with the number of CpG loci or sites selected for testing (i.e. the size of the signature), to a maximal sensitivity/specificity of 100%. However, signatures utilizing fewer CpG loci, are described herein which retain greater than 50% sensitivity and specificity and are useful for assessing likelihood of Kabuki syndrome.

Useful methylation signatures according to the described methods are not intended to be limited to the sites of Table 2, Table 16, Table 9 and Table 17, but are intended to include associated CpG loci, and associated gene and non-gene regions. DNA methylation at a single CpG locus can predict DNA methylation of multiple other loci residing in near genomic proximity or overlapping CpG islands. Accordingly, “associated” loci and regions are loci and regions, the methylation levels or status of which may be reasonably predicted by the methylation levels or status of one or more of the CpG loci of Table 2, Table 16, Table 9 and Table 17. CpG loci may be considered “associated”, for example, if they occur within the same CpG island, CpG island shore, gene promoter or gene enhancer region. CpG loci may also be considered “associated” by virtue of their proximity, for example, CpG loci residing within 300 nucleotides, optionally within 150 nucleotides, of each other may be considered associated.

Accordingly, an aspect of the disclosure provides a method of detecting and/or screening for CHARGE syndrome (CS), or an increased likelihood of CS, in a human subject, comprising determining a sample methylation profile from a sample of DNA from said subject, said sample profile comprising the methylation level of at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i).

Another aspect of the disclosure provides a method of detecting and/or screening for Kabuki syndrome (KS), or an increased likelihood of KS, in a human subject, comprising determining a sample methylation profile from a sample of DNA from said subject, said sample profile comprising the methylation level of at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i).

Methods of DNA methylation profiling of target genomic regions are generally known in the art (Stevens et al 2013, Harris et al 2010 and Hirst 2013).

For example, a non-limiting list of exemplary methods that may be used to determine methylation levels at a specified target sequence of

DNA include: bisulfite sequencing, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), methylation-sensitive restriction enzyme-based methods and/or microarray-based methods.

In an embodiment, methylation levels are measured using an agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG loci, and (b) an oligonucleotide comprising nucleotide sequence complementary to or identical to a nucleotide sequence of genomic DNA comprising the same selected CpG loci of (a), in which the cytosine residue of the CpG loci is replaced with a thymine residue. A non-limiting example of such an agent includes a “microarray”, comprising an ordered set of probes fixed to a solid surface that permits analysis such as methylation analysis of a plurality of genomic targets sequences.

According to the methods described herein, similarity of the DNA methylation profile from a sample to one or more control profiles, may be used to identify individuals having CHARGE syndrome, or an increased likelihood of having CHARGE syndrome. For example, in an embodiment, the method comprises determining the level of similarity of a sample profile to one or more control profiles, wherein (i) a high level of similarity of the sample profile to a CS specific profile; (ii) a low level of similarity to a non-CS control profile; and/or (iii) a higher level of similarity to a CS specific profile than to a non-CS control profile indicates the presence of, or an increased likelihood of, CS.

Similarity of the DNA methylation profile from a sample to one or more control profiles, may also be used to identify individuals having Kabuki syndrome, or an increased likelihood of having Kabuki syndrome. For example, in an embodiment, the method comprises determining the level of similarity of a sample profile to one or more control profiles, wherein (i) a high level of similarity of the sample profile to a KS specific profile; (ii) a low level of similarity to a non-KS control profile; and/or (iii) a higher level of similarity to a KS specific profile than to a non-KS control profile indicates the presence of, or an increased likelihood of, KS.

It will be appreciated that the control profile may be a reference value, or derived from one or more samples, optionally from historical methylation data for a patient or pool of patients. The control profile may be a reference value and/or may be derived from one or more samples, optionally from historical methylation data for a patient or pool of patients who are known to have, or not have, CHARGE syndrome and/or Kabuki syndrome. In such cases, the historical methylation data can be a value that is continually updated as further samples are collected and individuals are identified as CS or not-CS, or KS or not-KS. For example, the control database may be stored on an online database, which is continually updated with methylation data from diagnosed CS and non-CS patients and diagnosed KS and non-KS patients. It will be understood that the control profile represents an average of the methylation levels for selected CpG loci as described herein.

In an embodiment, the “CS specific control profile” is generated by measuring the methylation levels at specified target sequences in genomic DNA from an individual subject, or population of subjects, who are known to have CS. Similarly, in an embodiment, the “non-CS control profile” is generated by measuring the methylation levels at specified target sequences in genomic DNA from an individual subject, or population of subjects, who are known to not have CS. In certain embodiments, the tissue source from which the sample profile and control profile are derived is matched, so that they are both derived from the same or similar tissue. In other embodiments, the sample profile and control profile are derived from different tissues. In certain other embodiments, the CS specific control profile and the non-CS control profile are derived from historical data and can indicate similarity of a sample to either the CS or non-CS profiles.

In another embodiment, the “KS specific control profile” is generated by measuring the methylation levels at specified target sequences in genomic DNA from an individual subject, or population of subjects, who are known to have KS. Similarly, in an embodiment, the “non-KS control profile” is generated by measuring the methylation levels at specified target sequences in genomic DNA from an individual subject, or population of subjects, who are known to not have KS. In certain embodiments, the tissue source from which the sample profile and control profile are derived is matched, so that they are both derived from the same or similar tissue. In other embodiments, the sample profile and control profile are derived from different tissues. In certain other embodiments, the KS specific control profile and the non-KS control profile are derived from historical data and can indicate similarity of a sample to either the KS or non-KS profiles.

Methods of determining the similarity between methylation profiles are well known in the art. Methods of determining similarity may in some embodiments provide a non-quantitative measure of similarity, for example, using visual clustering. In another embodiment, similarity may be determined using methods which provide a quantitative measure of similarity.

For example, in an embodiment, similarity may be measured using hierarchical clustering, optionally using Manhattan distance. For example, unsupervised hierarchical clustering of a sample with a CS specific control profile indicates similarity to the CS specific control profile. Likewise, unsupervised hierarchical clustering of a sample with a non-CS control profile indicates similarity to the non-CS control profile. In another example, unsupervised hierarchical clustering of a sample with a KS specific control profile indicates similarity to the KS specific control profile. Likewise, unsupervised hierarchical clustering of a sample with a non-KS control profile indicates similarity to the non-KS control profile.

The Manhattan distance function computes the distance that would be traveled to get from one data point to the other if a grid-like path is followed. The Manhattan distance between two items is the sum of the differences of their corresponding components.

The formula for this distance between a point X=(X1, X2, etc.) and a point Y=(Y1, Y2, etc.) is:

$$d = {\sum }_{i = 1}^{n} \vert {x}_{i} - {y}_{i} \vert$$

Where n is the number of variables, and Xi and Yi are the values of the variable, at points X and Y respectively.

In another embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$\text{?} \text{?} \frac{{\sum }_{i} ( \text{?} - \text{?} ) ( \text{?} - \overline{y} )}{\sqrt{{\sum }_{i} {( \text{?} - \text{?} )}^{2}} \sqrt{{\sum }_{i} {( \text{?} - \overline{y} )}^{2}}}$$

$$\text{?} \text{indicates text missing or illegible when filed}$$

In one embodiment, x and y are the beta values for various CpG loci in a sample profile and a control profile, respectively.

In an embodiment, a correlation coefficient calculated between the sample profile and the control profile indicates a high level of similarity to the control profile when the correlation coefficient has an absolute value between 0.5 to 1, optionally between 0.75 to 1, and a low level of similarity to the control profile when the correlation coefficient has an absolute value between 0 to 0.5, optionally between 0 to 0.25.

It will be appreciated that any “correlation value” which provides a quantitative scaling measure of similarity between methylation profiles may be used to measure similarity. A sample profile may be identified as belonging to an individual with CS, or an increased likelihood of CS, where the sample profile has high similarity to the CS profile, low similarity to the non-CS profile, or higher similarity to the CS profile than to the non-CS profile. Conversely, a sample profile may be identified as belonging to an individual without CS, or a decreased likelihood of CS, where the sample profile has high similarity to the non-CS profile, low similarity to the CS profile, or higher similarity to the non-CS profile than to the CS profile.

For example, in an embodiment, a sample profile may be identified as belonging to an individual with CS, or an increased likelihood of CS, based on calculation of a CHARGE Syndrome Score, which generally is defined by the following formula:

CS score(B)=r (B, CS profile)−r (B, control profile)

where r is the Pearson correlation coefficient, and B is a vector of DNA methylation levels across the selected CpG loci.

A sample profile with a positive CHARGE Syndrome Score is more similar to the CS specific profile across the selected CpG loci, and is therefore classified as “CS”; whereas a sample with a negative CHARGE Syndrome Score is more similar to the non-CS profile across the selected CpG loci, and is classified as “not CS”.

In another embodiment, a sample profile may be identified as belonging to an individual with KS, or an increased likelihood of KS, where the sample profile has high similarity to the KS profile, low similarity to the non-KS profile, or higher similarity to the KS profile than to the non-KS profile. Conversely, a sample profile may be identified as belonging to an individual without KS, or a decreased likelihood of KS, where the sample profile has high similarity to the non-KS profile, low similarity to the KS profile, or higher similarity to the non-KS profile than to the KS profile.

For example, in an embodiment, a sample profile may be identified as belonging to an individual with KS, or an increased likelihood of KS, based on calculation of a Kabuki Syndrome Score, which generally is defined by the following formula:

KS score(B)=r (B, KS profile)−r (B, control profile)

where r is the Pearson correlation coefficient, and B is a vector of DNA methylation levels across the selected CpG loci.

A sample profile with a positive Kabuki Syndrome Score is more similar to the KS specific profile across the selected CpG loci, and is therefore classified as “KS”; whereas a sample with a negative Kabuki Syndrome Score is more similar to the non-KS profile across the selected CpG loci, and is classified as “not KS”.

As used herein the term “sample” refers to a biological sample comprising genomic DNA from a human subject. The sample may, for example, comprise blood, fibroblast tissue, buccal tissue, and/or amniotic fluid.

Median methylation levels for CS and non-CS cases reported in Tables 2 and/or 16 and for KS and non-KS reported in Tables 9 and/or 17 were identified using whole blood samples. Based on DNA methylation profiles in other disorders with mutations in epigenes, it is predicted that the

DNA methylation profile for CS and non-CS syndrome, and KS and non-KS, can be present in other samples, for example, fibroblast tissue, buccal tissue, lymphoblastoid cell lines, saliva or a prenatal sample. The prenatal sample is optionally a CVS, placenta, circulating fetal DNA and/or amniotic fluid sample.

Another aspect provides a method of detecting and/or screening for CHARGE syndrome (CS), or an increased likelihood of CS, in a human subject, comprising determining a sample DNA methylation profile from a sample of DNA from said subject, said sample profile comprising the methylation level of at least 2, optionally at least 3, at least 4, at least 6, at least 8, at least 10, at least 16, at least 20, at least 25, at least 30, at least 35, at least 40, or all the genes from Tables 2 and/or 16.

The method further comprises determining the level of similarity of said sample profile to one or more control profiles, wherein (i) a high level of similarity of the sample profile to a CS specific control profile; (ii) a low level of similarity to a non-CS control profile; and/or (iii) a higher level of similarity to a CS specific control profile than to a non-CS control profile indicates the presence of, or an increased likelihood of, CS.

Yet another aspect provides a method of detecting and/or screening for Kabuki syndrome (KS), or an increased likelihood of KS, in a human subject, comprising determining a sample DNA methylation profile from a sample of DNA from said subject, said sample profile comprising the methylation level of at least 3, optionally at least 4, at least 6, at least 8, at least 10, at least 15, at least 20, at least 25, at least 50, at least 75, at least 100, at least 125, or all the genes from Tables 9 and/or 17.

In one embodiment, the genes are FAM65B, HOXC4 and MYO1F. It is shown in Table 15, for example, that at an absolute delta beta of 0.25 and p-value 0.00001, the three genes FAM65B, HOXC4 and MYO1F provide a specificity of 100% and a sensitivity of 90.9%.

The method further comprises determining the level of similarity of said sample profile to one or more control profiles, wherein (i) a high level of similarity of the sample profile to a KS specific control profile; (ii) a low level of similarity to a non-KS control profile; and/or (iii) a higher level of similarity to a KS specific control profile than to a non-KS control profile indicates the presence of, or an increased likelihood of, KS.

It will also be appreciated by a person of skill in the art that the methods described herein can be used to distinguish between CHARGE syndrome and other neurodevelopmental syndromes such as Kabuki syndrome. Further, the methods described herein can be used to distinguish between Kabuki syndrome and other neurodevelopmental syndromes such as CHARGE syndrome.

While both CHARGE syndrome and Kabuki syndrome share some characteristics such as developmental delay, cardiovascular malformations, growth deficiency, orofacial clefts, genitourinary anomalies, including cryptorchidism in males, seizures and hearing loss (there can be different causes for each condition), there are also clinical characteristics that are typical of CHARGE syndrome and not Kabuki syndrome and vice versa.

For example, clinical characteristics typical of CHARGE Syndrome, but not Kabuki syndrome, include, but are not limited to: unilateral or bilateral coloboma of the iris, retina-choroid, and/or disc with or without microphthalmos (80%-90% of individuals); unilateral or bilateral choanal atresia or stenosis (50%-60%); cranial nerve dysfunction resulting in hyposmia or anosmia, unilateral or bilateral facial palsy (40%), impaired hearing, and/or swallowing problems (70%-90%); and abnormal outer ears, ossicular malformations, Mondini defect of the cochlea and absent or hypoplastic semicircular canals (>90%).

Further, clinical characteristics typical of Kabuki Syndrome, but not CHARGE syndrome, include, but are not limited to: skeletal anomalies; spinal column abnormalities, including sagittal cleft vertebrae, butterfly vertebrae, narrow intervertebral disc space, and/or scoliosis; hypodontia;

susceptibility to infections and autoimmune disorders; gastrointestinal anomalies, including anal atresia; and ophthalmologic anomalies, including ptosis and strabismus.

Therefore, a proper diagnosis of CHARGE syndrome or Kabuki syndrome allows for testing, treatment and medical management appropriate for each condition, given the differences in their clinical characteristics.

Accordingly, the present disclosure provides a method of detecting and/or screening for CHARGE syndrome (CS) or Kabuki syndrome (KS), or an increased likelihood of CS or KS, in a human subject, comprising:
determining a sample methylation profile from a sample comprising DNA from said subject, said sample profile comprising (a) the methylation level of at least 3, optionally at least 5, at least 8, at least 10, at least 25, at least 44, at least 50, at least 75, at least 100, at least 125, at least 140, or all

CpG loci from (i) Tables 2 and/or 16 and/or (ii) associated CpG loci residing within 300 nucleotides, optionally within 150 nucleotides, of the CpG loci of (i); and (b) the methylation level of at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and
determining the level of similarity of said sample profile to one or more control profiles, wherein (i) a high level of similarity of the sample profile to a CS specific control profile; (ii) a low level of similarity to a KS specific control profile; and/or (iii) a higher level of similarity to a CS specific control profile than to a KS specific control profile indicates the presence of, or an increased likelihood of, CS and/or wherein (i) a high level of similarity of the sample profile to a KS specific control profile; (ii) a low level of similarity to a CS specific control profile; and/or (iii) a higher level of similarity to a KS specific control profile than to a CS specific control profile indicates the presence of, or an increased likelihood of, KS.

The disclosure also provides a method of distinguishing between CHARGE syndrome (CS) or Kabuki syndrome (KS), or an increased likelihood of CS or KS, in a human subject, comprising:
(A) determining a sample methylation profile from a sample comprising DNA from said subject, said sample profile comprising the methylation level of at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i); and
determining the level of similarity of said sample profile to one or more control profiles, wherein (i) a high level of similarity of the sample profile to a CS specific control profile; (ii) a low level of similarity to a non-CS control profile; and/or (iii) a higher level of similarity to a CS specific control profile than to a non-CS control profile indicates the presence of, or an increased likelihood of, CS, and
(B) determining a second sample methylation profile from a sample comprising DNA from said subject, said second sample profile comprising the methylation level of at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and
determining the level of similarity of said second sample profile to one or more control profiles, wherein (i) a high level of similarity of the second sample profile to a KS specific control profile; (ii) a low level of similarity to a non-KS control profile; and/or (iii) a higher level of similarity to a KS specific control profile than to a non-KS control profile indicates the presence of, or an increased likelihood of, KS.

Confirmation of a diagnosis of CHARGE aids in medical management by enabling targeted screening for the multisystem manifestations of this complex condition, optimizing the opportunity for early intervention and management. Recommended evaluations following a diagnosis include: ophthalmology exam to look for colobomas, cardiac exam to screen for cardiovascular anomalies, audiology exam to assess for hearing loss, airway evaluation (risk for choanal atresia/stenosis and tracheoesophageal fistula) and feeding evaluation (aspiration/swallowing dysfunction common due to abnormalities of cranial nerve IX/X). Individuals with CHARGE syndrome will require ongoing ophthalmology follow-up, as they may have an increased risk for retinal detachment, and audiology follow-up for management of hearing loss. Individuals with CHARGE syndrome should be followed by endocrinology as growth delay is usually evident by late infancy and may require investigation/management. In addition individuals with CHARGE syndrome are at increased risk for delayed puberty as a result of hypogonadotropic hypogonadism for which they require ongoing monitoring. In light of the increased risk of renal anomalies, a renal ultrasound should be done. In addition, neuropsychological assessment to screen for developmental difficulties (highly prevalent) and behavioural problems (e.g. aggression, obsessive-compulsive behaviors) provides the opportunity for early identification and intervention. Individuals with CHARGE syndrome are at increased risk for dual sensory loss (hearing and vision). There is also an increased risk for other neuropsychological issues including attention deficit hyperactivity disorder and autism—early diagnosis provides the opportunity for early intervention and improved outcomes. Early identification of the above medical and cognitive issues provides the opportunity for an enhanced quality of life for individuals with CHARGE syndrome.

Similarly, confirmation of a diagnosis of Kabuki syndrome aids in medical management by enabling targeted screening for the multisystem manifestations of this complex condition, optimizing the opportunity for early intervention and management. Recommended evaluations following a diagnosis include: ophthalmology exam to look for strabisimus and ptosis, cardiac exam to screen for cardiovascular anomalies, audiology exam to assess for hearing loss, abdominal ultrasound to screen for kidney abnormalities, x-rays for skeletal anomalies, dental assessment for missing teeth and feeding evaluation for gastrosophageal reflux and gastrostomy tube placement if feeding difficulties are severe. Prophylactic antibiotic treatment prior to and during any procedure (e.g. dental work) may be indicated for those with specific heart defects. Individuals with Kabuki syndrome will require ongoing endocrine assessment for various endocrine problems including isolated premature thelarche, ophthalmology follow-up if strabismus or ptosis are present, and audiology follow-up for management of hearing loss. In addition, individuals with Kabuki syndrome require ongoing follow-up for their increased risks for infections and autoimmune disorders as well as seizures In addition, neuropsychological assessment to screen for developmental difficulties (highly prevalent) and autism provides the opportunity for early identification and intervention. Early identification of the above medical and cognitive issues provides the opportunity for an enhanced quality of life for individuals with Kabuki syndrome.

Accordingly, an aspect of the disclosure provides a method of assigning a course of management for an individual with CHARGE syndrome (CS), or an increased likelihood of CS, comprising:
a) identifying an individual with CS or an increased likelihood of CS, according to the methods described herein; and
b) assigning a course of management for CS and/or symptoms of CS, comprising i) testing for at least one medical condition associated with CS and ii) applying an appropriate medical intervention based on the results of the testing.

Another aspect of the disclosure provides a method of assigning a course of management for an individual with Kabuki syndrome (KS), or an increased likelihood of KS, comprising:
a) identifying an individual with KS or an increased likelihood of KS, according to the methods described herein; and
b) assigning a course of management for KS and/or symptoms of KS, comprising i) testing for at least one medical condition associated with KS and ii) applying an appropriate medical intervention based on the results of the testing.

As used herein, the term “a course of management” refers to the any testing, treatment, medical intervention and/or therapy applied to an individual with CS or KS and/or symptoms of CS or KS. Medical interventions include, but are not limited to, pharmaceutical treatments, surgical procedures, utilization of medical devices such as hearing aids or glasses, physical or occupational therapy and behavioral or cognitive therapy.

In one embodiment, the medical condition associated with CS is selected from ophthalmic colobomas, cardiovascular anomalies, hearing loss, airway conditions such as choanal atresia/stenosis or tracheoesophageal fistula, feeding issues, retinal detachment, growth delay, delayed puberty, renal anomalies, developmental difficulties, behavioural problems, dual sensory loss and neuropsychological issues such as attention deficit hyperactivity disorder or autism. Other medical conditions associated with CS include, but are not limited to, developmental delay, cardiovascular malformations, growth deficiency, orofacial clefts, genitourinary anomalies, including cryptorchidism in males, seizures and hearing loss, unilateral or bilateral coloboma of the iris, retina-choroid, and/or disc with or without microphthalmos, unilateral or bilateral choanal atresia or stenosis, cranial nerve dysfunction resulting in hyposmia or anosmia, unilateral or bilateral facial palsy, impaired hearing, and/or swallowing problems, abnormal outer ears, ossicular malformations, Mondini defect of the cochlea and absent or hypoplastic semicircular canals.

In another embodiment, the medical condition associated with KS is selected from ophthalmic abnormalities, cardiovascular anomalies, hearing loss, kidney abnormalities, skeletal anomalies, dental abnormalities, feeding difficulties, endocrine problems, infection, autoimmune disorders, seizures and developmental difficulties such as autism. Other medical conditions associated with KS include, but are not limited to, developmental delay, cardiovascular malformations, growth deficiency, orofacial clefts, genitourinary anomalies, including cryptorchidism in males, seizures and hearing loss, skeletal anomalies, spinal column abnormalities, including sagittal cleft vertebrae, butterfly vertebrae, narrow intervertebral disc space, and/or scoliosis, hypodontia, susceptibility to infections and autoimmune disorders, gastrointestinal anomalies, including anal atresia; and ophthalmologic anomalies, including ptosis and strabismus.

### III. Kits

Another aspect provides a kit for detecting and/or screening for CHARGE syndrome (CS), or an increased likelihood of CS, in a sample, comprising:

(a) at least one detection agent for determining the methylation level of:
at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i), and;

(b) instructions for use.

Another aspect provides a kit for detecting and/or screening for CHARGE syndrome (CS), or an increased likelihood of CS, in a sample, comprising:

(a) at least one detection agent for determining the methylation level of:
at least 2, optionally at least 3, at least 4, at least 6, at least 8, at least 10, at least 16, at least 20, at least 25, at least 30, at least 35, at least 40, or all the genes from Tables 2 and/or 16 and;

(b) instructions for use.

Another aspect provides a kit for detecting and/or screening for Kabuki syndrome (KS), or an increased likelihood of KS, in a sample, comprising:

(a) at least one detection agent for determining the methylation level of:
at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i), and;

(b) instructions for use.

Another aspect provides a kit for detecting and/or screening for Kabuki syndrome (KS), or an increased likelihood of KS, in a sample, comprising:

(a) at least one detection agent for determining the methylation level of:
at least 3, optionally at least 4, at least 6, at least 8, at least 10, at least 15, at least 20, at least 25, at least 50, at least 75, at least 100, at least 125, or all the genes from Tables 9 and/or 17 and;

(b) instructions for use.

In an embodiment, the kit further comprises bisulfite conversion reagents, methylation-dependent restriction enzymes, methylation-sensitive restriction enzymes, PCR reagents, probes and/or primers.

In another embodiment, the kit further comprises a computer-readable medium that causes a computer to compare methylation levels from a sample at the selected genes to one or more control profiles and compute a correlation value between the sample and control profile.

In another embodiment, the kit further comprises a computer-readable medium that causes a computer to compare methylation levels from a sample at the selected CpG loci to one or more control profiles and compute a correlation value between the sample and control profile.

Other features and advantages of the disclosure will become apparent from the following detailed description. It should be understood, however, that the description and the specific examples while indicating preferred embodiments are given by way of illustration only, since various changes and modifications within the spirit and scope of the disclosure will become apparent to those skilled in the art from this description of various embodiments.

### EXAMPLES

### Example 1

DNA methylation was determined in the blood of subjects with CHARGE and a nonsense mutation in CHD7 compared to controls. A set of CpG sites that can be used as a signature to distinguish subjects from controls was identified. This set of CpG sites can be used to distinguish patients from controls and determine if a variant in CHD7 is mostly likely pathogenic or benign. This signature was also specific to those subjects compared to a large sample of population controls. Many of the CpG sites with greater than 10% differences in DNA methylation are known to play a role in early embryonic growth and development. The DNA methylation alterations that occur as a result of heterozygous CHD7 mutations also reveal genes, such as those in the HOXA cluster and FOXP2, which may play a critical role in the aberrant development associated with the clinical spectrum of CHARGE syndrome.

### Subjects and Methods

### Subjects and Clinical Information

Individuals with a clinical diagnosis of CHARGE syndrome, who meet the clinical criteria of Blake²³ or Verloes³⁰, were recruited through the Division of Clinical and Metabolic Genetics at the Hospital for Sick Children in

Toronto. DNA methylation of whole blood was analyzed in 15 DNA samples from individuals with CHD7 pathogenic nonsense mutations. An additional 14 subjects with variants in CHD7 including missense, splice site missense, variants of unknown significance (VUS) in CHD7 that have a clinical diagnosis of CHARGE syndrome and 4 with sequence variants in CHD7 without CHARGE syndrome (Table 1) were compared to 45 age, sex and ethnicity matched controls. Phenotypic information was available for all of the subjects. The control subjects and those with missense mutations in CHD7 were recruited through The Hospital for Sick Children.

All subjects were recruited following informed consent. The study was approved by the Research Ethics Boards of the Hospital for Sick Children Toronto. DNA was extracted from whole blood collected from cases and controls.

Control DNA Methylation Data from Public Databases

Publically available HumanMethylation450 data at the GEO resource DNA methylation data for an additional 1056 control blood samples were downloaded from the GEO public database (http://www.ncbi.nlm.nih.gov/sites/GDSbrowser/).

### Methylation Array Analysis

DNA samples were modified using sodium bisulfite (EpiTect PLUS Bisulfite Kit, QIAGEN). The sodium bisulfite converted DNA was then hybridized to the Illumina Infinium HumanMethylation450 BeadChip Array to interrogate over 485577 CpG sites in the human genome. Illumina Genome studio software was used to extract DNA methylation values (β values), calculated after control probe normalization and background subtraction using the formula C/(C+T), and ranging between 0 (no methylation) and 1 (full methylation). Autosomal probes that cross-react with sex chromosome probes, non-specific probes, and probes targeting CpG sites at a known SNP^{31,32} were excluded. The analysis was performed on the remaining 432,601 probes. Since for most CpG sites across the genome DNA methylation is not normally distributed, the non-parametric test to determine changes in DNA methylation between groups was used. For each probe, Mann-Whitney U test was performed to compare 21 blood samples from subjects with a known CHD7 pathogenic mutation samples to 45 controls, followed by the Benjamini-Hochberg correction for multiple testing.

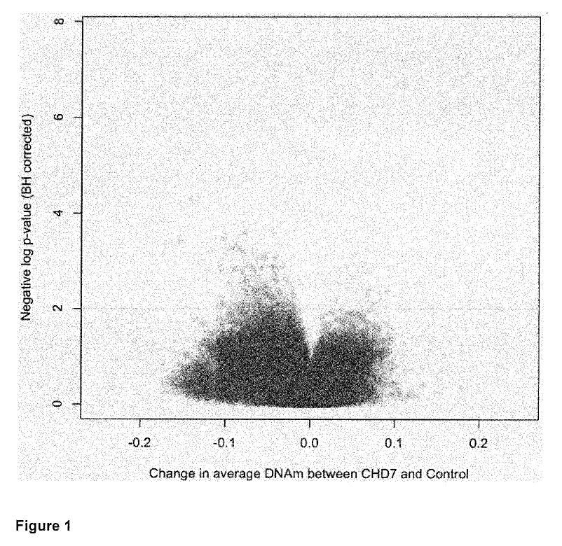

To determine the appropriate significance level for the Mann-Whitney U tests, the volcano plot (FIG. 1) was first examined, which suggested the p-value threshold 0.01. This p-value threshold was confirmed by a series of leave-one-out (LOO) cross-validations on the combined dataset. In each LOO iteration, one sample was removed from the dataset for the subsequent validation step (Tables 3-6). The remaining samples were used to generate median DNA methylation profiles for the subjects containing a CHD7 mutation group and for the control group, respectively. The retained validation sample was then compared to both reference profiles, using only the significant CpGs, and with Pearson correlation as the measure of similarity. The sample was assigned to the group with the more similar profile, and the assignment compared to the true status of the sample (those with a CHD7 nonsense mutation or control). Iterating the LOO process over all 60 samples, the classification accuracy was estimated in terms of the specificity and sensitivity for a given level of significance. To ensure robust results, statistically significant probes were additionally filtered for the effect size. Delta beta (Δβ) was defined for each probe as the difference between average control and average CHD7 nonsense mutation methylation levels (Tables 2 and/or 16). Only those significant probes for which the DNA methylation difference (Δβ) was greater than an absolute value of 0.10 were retained. Statistical analysis was performed in R using custom scripts.

### Results

### CHD7 Signature

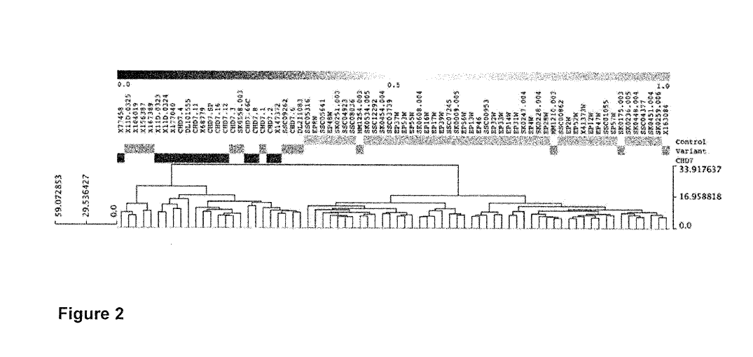

The LOO procedure confirmed that the p-value threshold 0.01, when combined with the effect size threshold |Δβ|>0.10, was the necessary significance level at which the LOO procedure makes no classification errors. Applying the statistical tests with these parameters to the full collection of 15 CHD7 nonsense mutation samples and 45 controls, a “signature set” of 146 significant CpG sites was derived. As expected, the set defined a perfect separation between the samples with a pathogenic CHD7 mutation and controls (FIG. 2).

### Signature Validation

The resulting set of probes for specific CpG sites were located within the bodies or promoter regions of 44 known genes (Table 2). Several genes had more than one differentially methylated CpG site including FOXP2, HOTAIRM1, SLITRKS and multiple genes in the HOXA cluster. Enrichment analysis of the resulting set using DAVID (http://david.abcc.ncifcrf.gov/) confirmed a statistically significant over-representation in genes related to skeletal, neural and lung development, as well as to transcriptional regulation.

These functional categories are highly relevant to the CHARGE syndrome phenotype, validating the biological importance of the derived DNA methylation signature.

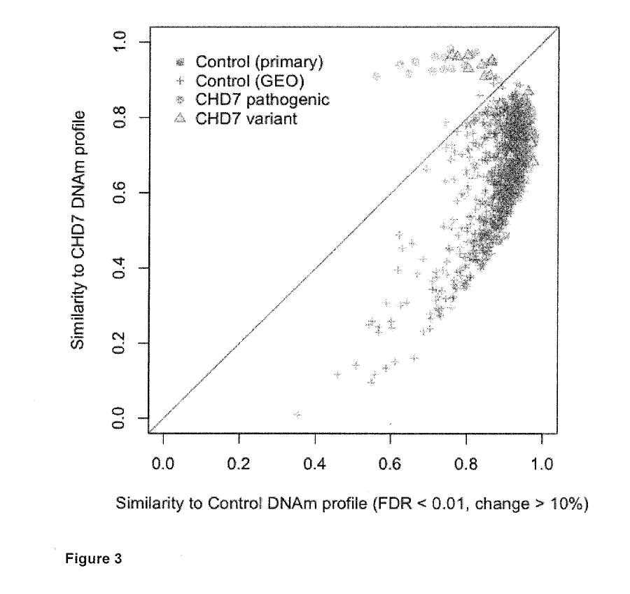

Next the specificity of the signature CpGs on a collection of 1056 normal blood samples derived from GEO was validated. Similar to the LOO procedure, median DNAm profiles for the 15 CHD7 nonsense mutation samples and for the 45 control samples, respectively, were generated. The Pearson correlation of each of the GEO samples with the reference CHD7 profile and the reference control profiles, using the 146 significant CpGs sites was computed. Only 5 samples exhibited a higher correlation with the CHD7 profile, whereas the remaining 1047 samples were classified as normal, resulting in 99.5% specificity (FIG. 3). This high specificity estimate is encouraging, given the diversity and unknown phenotype of the combined data from GEO sources. Similar estimates were tabulated for additional parameter combinations for effect size threshold |Δβ| from 5% to 22% and significance level from p<0.01 to 0.00005 (Tables 3-6).

The signature was then applied to classify 14 subjects with CHD7 mutation that did not result in a nonsense mutation into either pathogenic or benign mutations (FIG. 3). Using the same classification procedure as was used to define the signature, 9 of the variants were predicted to be pathogenic, whereas the remaining samples were predicted to be benign.

### Example 2

### Summary

To date, approximately two-thirds of Kabuki syndrome patients have an identified mutation in the Lysine (K) Methyltransferase 2D (KMT2D) gene. Mutations in KMT2D may cause downstream alterations in DNA methylation (DNAm), a modification of DNA that can alter gene expression without modifying the DNA sequence itself.

DNA methylation was determined in the blood of subjects with Kabuki syndrome and a nonsense mutation in KMT2D compared to controls and is set of CpG sites that could be used as a signature to distinguish subjects from controls were identified. This set of CpG sites is used to distinguish patients from controls and determine if a variant in KMT2D is pathogenic or benign. This signature is also specific to those subjects compared to a large sample of population controls. Many of the CpG sites with greater than 15% differences in DNA methylation are known to play a role in early embryonic growth and development. The DNA methylation alterations that occur as a result of heterozygous KMT2D mutations also reveal genes, such as those in the HOXA cluster, laminin beta 2 (LAMB2) and myosin F1 (MYOF1), which may play a critical role in the aberrant development associated with the clinical spectrum of Kabuki syndrome.

### Subjects and Methods

### Subjects and Clinical Information

Individuals with a clinical diagnosis of Kabuki syndrome³⁶ were recruited through the Division of Clinical and Metabolic Genetics at the Hospital for Sick Children in Toronto, or the Center for Human Genetics, Inc., Cambridge, USA. DNA methylation of whole blood was analyzed in 11 DNA samples from individuals with KMT2D pathogenic nonsense mutations. An additional 9 subjects with variants in KMT2D including 1 missense mutation, 1 variant of unknown significance (VUS) in KMT2D that has a clinical diagnosis of Kabuki syndrome and 6 with missense variants in KMT2D without Kabuki syndrome (Table 7) compared to 45 age, sex and ethnicity matched controls. There was also one additional subject that had a diagnosis of Kabuki syndrome but the mutation status was not known at the time of analysis. The control subjects and those with missense mutations in KMT2D were recruited through The Hospital for Sick Children and Simons Simplex Collection³⁷.

All subjects were recruited following informed consent. DNA was extracted from whole blood collected from cases and controls.

Control DNA Methylation Data from Public Databases

Publically available HumanMethylation450 data at the GEO resource DNA methylation data for an additional 1056 control blood samples were downloaded from the GEO public database (http://www.ncbi.nlm.nih.gov/sites/GDSbrowser/).

### Methylation Array Analysis

DNA samples were modified using sodium bisulfite (EpiTect PLUS Bisulfite Kit, QIAGEN). The sodium bisulfite converted DNA was then hybridized to the Illumina Infinium HumanMethylation450 BeadChip Array to interrogate over 485,577 CpG sites in the human genome. Illumina Genome studio software was used to extract DNA methylation values (β values), calculated after control probe normalization and background subtraction using the formula C/(C+T), and ranging between 0 (no methylation) and 1 (full methylation). Autosomal probes that cross-react with sex chromosome probes, non-specific probes, and probes targeting CpG sites at a known SNP^{38,39} were excluded. The analysis was performed on the remaining 422, 139 probes. Since for most CpG sites across the genome DNA methylation is not normally distributed, the non-parametric test was used to determine changes in DNA methylation between groups. For each probe, Mann-Whitney

U test was performed to compare 11 blood samples from subject with a known KMT2D pathogenic mutation samples and 45 controls, followed by the Benjamini-Hochberg correction for multiple testing.

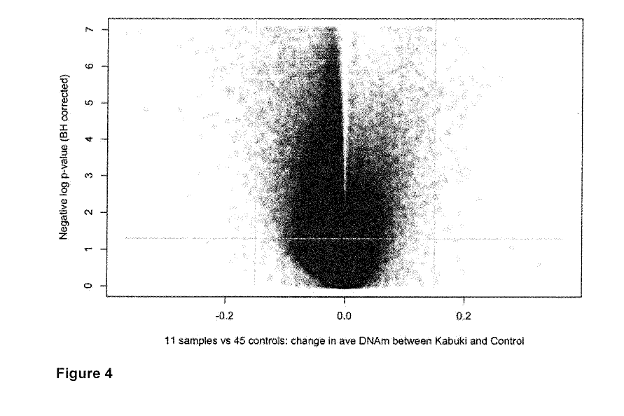

To determine the appropriate significance level for the Mann-Whitney U tests, the volcano plot (FIG. 4) was first examined, which suggested that the p-value threshold 0.05. This p-value threshold was confirmed by a series of leave-one-out (LOO) cross-validations on the combined dataset. In each LOO iteration one sample was removed from the dataset for the subsequent validation step (Table 8). The remaining samples were used to generate median DNA methylation profiles for the subjects containing a KMT2D mutation group and for the control group, respectively. The retained validation sample was then compared to both reference profiles, using only the significant CpGs, and with Pearson correlation as the measure of similarity. The sample was assigned to the group with the more similar profile, and the assignment compared to the true status of the sample (those with a KMT2D nonsense mutations or control). Iterating the LOO process over all 56 samples, the classification accuracy was estimated in terms of the specificity and sensitivity for a given level of significance. To ensure robust results, statistically significant probes were additionally filtered for the effect size. Delta beta (Δβ) was defined for each probe as the difference between average control and average KMT2D nonsense mutation methylation levels (Table 3). Only those significant probes for which the DNA methylation difference (Δβ) was greater than an absolute value of 15% were retained. Statistical analysis was performed in R using custom scripts.

### Results

### KMT2D Signature

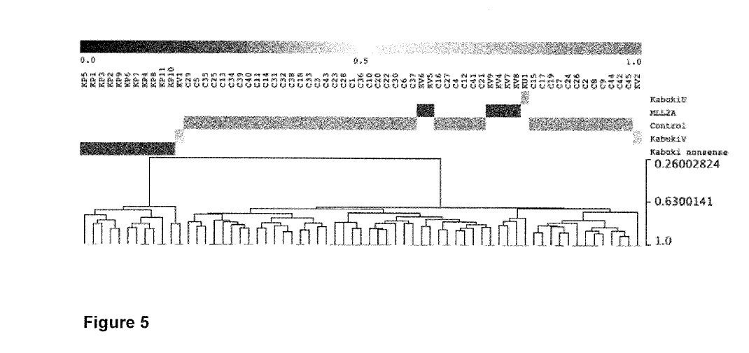

The LOO procedure confirmed that the p-value threshold 0.05, when combined with the effect size threshold |Δβ|>15%, was the necessary significance level at which the LOO procedure makes no classification errors (see Table 8). Applying the statistical tests with these parameters to the full collection of 11 KMT2D nonsense mutation samples and 45 controls, a “signature set” of 287 significant CpG sites was derived. As expected, the set defined a perfect separation between the samples with a pathogenic KMT2D mutation and controls (FIG. 5).

The resulting set of probes for specific CpG sites were located within the bodies or promoter regions of 162 known genes (Table 9). Several genes had more than one differentially methylated CpG site including LAMB2, MYO1F, AGAP2 ArfGAP with GTPase domain, ankyrin repeat and PH domain 2 and multiple genes in the HOXA cluster, with the most probes differentially methylated in HOXA4. An additional 28 genes (Table 17) have been identified that include a muscle specific isoform CPT1B, which had more than one differentially methylated CpG site.

Next, the specificity of the signature CpGs on a collection of 1056 normal blood samples derived from GEO was validated. Similar to the LOO procedure, median DNAm profiles for the 11 KMT2D nonsense mutation samples and for the 45 control samples, respectively, were generated. The Pearson correlation of each of the GEO samples with the reference KMT2D profile and the reference control profiles, using the 287 significant CpGs sites. None of these samples exhibited a higher correlation with the KMT2D profile therefore there was a 100% specificity (FIG. 5). This high specificity estimate is encouraging, given the diversity and unknown phenotype of the combined data from GEO sources. Similar estimates were tabulated for additional parameter combinations for effect size threshold |Δβ| from 5% to 25% and significance level from p<0.01 to 0.00001 (Tables 10-15).

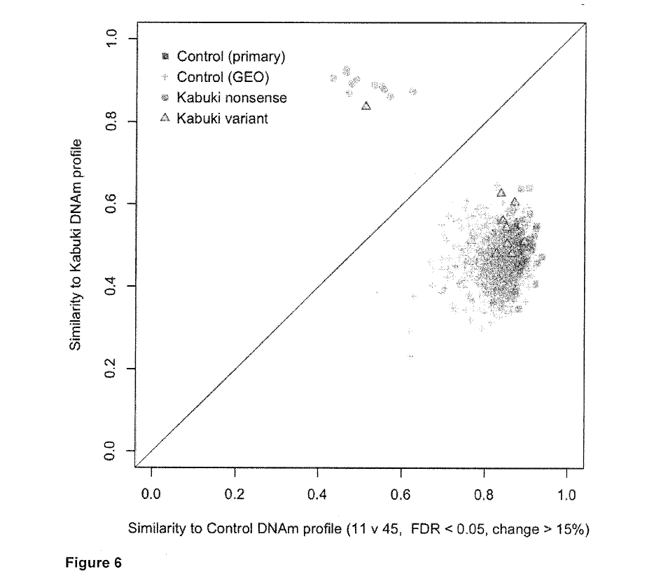

The signature was then applied to classify 9 subjects with KMT2D mutation that did not result in a nonsense mutation into either pathogenic or benign mutations (FIG. 6). Using the same classification procedure as was used to define the signature, 1 of the variants was predicted to be pathogenic, whereas the remaining samples were predicted to be benign, including the subject for which molecular testing is still pending (Table 8). There was a high correlation between the clinical phenotype and the corresponding KMT2D-specific DNA methylation profile.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
CHD7 mutation information for all cases

| | | | | Mutation |
|---|---|---|---|---|
| Sample ID | Sex | Nucleotide | Protein | Type |
| CHD7-16 | F | c.7282C > T | p.Arg2428X | nonsense |
| 77458 | M | c.3526C > T | p.Gln1176X | nonsense |
| CHD7-2 | F | c.934C > T | p.Arg312X | nonsense |
| 147372 | M | c562C > T | p.Gly188X | nonsense |
| CHD7-66C | M | c.1327delATGGG | p.Met443Asnfs*130 | deletion |
| CHD7-12 | M | c.2504_2508delATCTT | p.Tyr835Serfs*14 | deletion |
| 11D/0324 | M | c.1990G > T | p.Glu664X | nonsense |
| 68779 | F | c.3377dupT | p.Leu1126fs*46 | duplication |
| CHD7-4 | M | c.2585delA | p.Lys862Serfs*26 | deletion |
| 177040 | F | c.2905_2906del | p.Arg969Glyfs*25 | deletion |
| SP-CHD7 | M | c.7636G > T | p.Glu2546X | nonsense |
| CHD7-8 | M | c.361delC | p.Gly121Valfs*90 | deletion |
| CHD7-11 | M | c.2504_2508delATCTT | p.Tyr835Serfs*14 | deletion |
| 11D/0323 | M | c.7717-7720del | p.Glu2537X | nonsense |
| DL101555 | M | c.5458C > T | p.Arg1820X | nonsense |

TABLE 2
146 CpG loci corresponding to 44 genes were identified as showing a statistically significant (corrected
p-value <0.01) difference in CS and non-CS controls.

| | | Benjamini- | | | | | | |
|---|---|---|---|---|---|---|---|---|
| | | Hochberg | | | DNA | | | |
| | | corrected p- | | Absolute | methylation | Mean not- | Mean | |
| Illumine ID | p-value | value | deltaBeta | deltaBeta | effect | CHARGE | CHARGE | Gene Symbol |
| cg17569124 | 2.63E−13 | 2.84638E−08 | 0.24269876 | 0.24269876 | GAIN | 0.628406184 | 0.87110494 | HOXA5; HOXA- |
| | | | | | | | | AS3 |
| cg25307665 | 7.14E−13 | 4.41479E−08 | 0.2380737 | 0.2380737 | GAIN | 0.659500102 | 0.8975738 | HOXA5; HOXA- |
| | | | | | | | | AS3 |
| cg12128839 | 3.65E−12 | 7.88853E−08 | 0.22084216 | 0.22084216 | GAIN | 0.62802854 | 0.8488707 | HOXA5; HOXA5; HOXA- |
| | | | | | | | | AS3 |
| cg05076221 | 1.4E−11 | 1.95705E−07 | 0.21817837 | 0.21817837 | GAIN | 0.571198187 | 0.78937656 | HOXA5; HOXA- |
| | | | | | | | | AS3 |
| cg19759481 | 1.69E−12 | 6.00554E−08 | 0.19441479 | 0.19441479 | GAIN | 0.68921206 | 0.883626853 | HOXA5; HOXA5; HOXA- |
| | | | | | | | | AS3 |
| cg04863892 | 1.5E−13 | 2.84638E−08 | 0.1890898 | 0.1890898 | GAIN | 0.686186942 | 0.875276747 | HOXA5; HOXA5; HOXA- |
| | | | | | | | | AS3 |
| cg04053108 | 6.9E−09 | 3.31436E−05 | 0.18874139 | 0.18874139 | GAIN | 0.248886338 | 0.437627727 | VWF |
| cg02005600 | 2.52E−12 | 6.41032E−08 | 0.17852388 | 0.17852388 | GAIN | 0.705460591 | 0.883984473 | HOXA5; HOXA- |
| | | | | | | | | AS3 |
| cg23936031 | 1.8E−12 | 6.00554E−08 | 0.17634521 | 0.17634521 | GAIN | 0.765829049 | 0.942174257 | HOXA5; HOXA5; HOXA- |
| | | | | | | | | AS3 |
| cg09319828 | 1.24E−05 | 0.008204599 | 0.17363223 | 0.17363223 | GAIN | 0.324044113 | 0.497676347 | TTC24 |
| cg02916332 | 1.13E−12 | 5.42167E−08 | 0.17187598 | 0.17187598 | GAIN | 0.651970609 | 0.823846587 | HOXA5; HOXA- |
| | | | | | | | | AS3 |
| cg03368099 | 6.9E−09 | 3.31436E−05 | 0.16690336 | 0.16690336 | GAIN | 0.564462407 | 0.731365767 | HOXA5; HOXA- |
| | | | | | | | | AS3 |
| cg11724970 | 5.23E−12 | 9.42015E−08 | 0.16262001 | 0.16262001 | GAIN | 0.74328516 | 0.905905167 | HOXA5; HOXA- |
| | | | | | | | | AS3 |
| cg18274664 | 3.76E−14 | 1.6265E−08 | 0.15866987 | 0.15866987 | GAIN | 0.595106733 | 0.753776607 | APP; APP; APP; APP |
| cg03529432 | 1.3E−10 | 1.25096E−06 | 0.15645583 | 0.15645583 | GAIN | 0.104669809 | 0.26112564 | HOXA6; HOXA6; HOXA- |
| | | | | | | | | AS3; HOXA- |
| | | | | | | | | AS3 |
| cg05835726 | 3.02E−12 | 7.26996E−08 | 0.15449369 | 0.15449369 | GAIN | 0.74016994 | 0.894663627 | HOXA5; HOXA- |
| | | | | | | | | AS3 |
| cg02248486 | 1.69E−12 | 6.00554E−08 | 0.15406701 | 0.15406701 | GAIN | 0.725477107 | 0.87954412 | HOXA5; HOXA5; HOXA- |
| | | | | | | | | AS3 |
| cg17432857 | 3.65E−12 | 7.88853E−08 | 0.15398978 | 0.15398978 | GAIN | 0.650043193 | 0.804032973 | HOXA5; HOXA- |
| | | | | | | | | AS3 |
| cg14882265 | 2.57E−11 | 3.27214E−07 | 0.15191829 | 0.15191829 | GAIN | 0.734545273 | 0.886463567 | HOXA5; HOXA- |
| | | | | | | | | AS3 |
| cg11321156 | 7.14E−13 | 4.41479E−08 | 0.14905949 | 0.14905949 | GAIN | 0.598582929 | 0.74764242 | APP; APP; APP; APP; |
| | | | | | | | | APP; APP; APP; AP |
| cg01370449 | 7.14E−13 | 4.41479E−08 | 0.14892232 | 0.14892232 | GAIN | 0.72699072 | 0.87591304 | HOXA5; HOXA5; HOXA- |
| | | | | | | | | AS3 |
| cg20517050 | 1.4E−11 | 1.95705E−07 | 0.14656577 | 0.14656577 | GAIN | 0.73752756 | 0.884093327 | HOXA5; HOXA- |
| | | | | | | | | AS3 |
| cg14044640 | 1.01E−10 | 1.09301E−06 | 0.14640823 | 0.14640823 | GAIN | 0.043525592 | 0.189933825 | HOXA6; HOXA6; HOXA- |
| | | | | | | | | AS3; HOXA- |
| | | | | | | | | AS3 |
| cg23269692 | 5.23E−12 | 9.42015E−08 | 0.14592414 | 0.14592414 | GAIN | 0.631808842 | 0.777732987 | APP; APP; APP; APP; |
| | | | | | | | | APP; APP; APP; AP |
| cg23129930 | 1.16E−08 | 4.88234E−05 | 0.14555376 | 0.14555376 | GAIN | 0.599694249 | 0.745248013 | HOXA6; HOXA- |
| | | | | | | | | AS3; HOXA-AS3 |
| cg26023912 | 4.55E−11 | 5.6184E−07 | 0.1445049 | 0.1445049 | GAIN | 0.694275447 | 0.838780347 | HOXA5; HOXA- |
| | | | | | | | | AS3 |
| cg25866143 | 7.33E−12 | 1.21988E−07 | 0.13778995 | 0.13778995 | GAIN | 0.751083682 | 0.888873633 | HOXA5; HOXA5; HOXA- |
| | | | | | | | | AS3 |
| cg06237983 | 6.9E−09 | 3.31436E−05 | 0.13205043 | 0.13205043 | GAIN | 0.333813007 | 0.46586344 | HOXA6; HOXA6; HOXA- |
| | | | | | | | | AS3; HOXA- |
| | | | | | | | | AS3 |
| cg02646423 | 1.02E−11 | 1.58003E−07 | 0.13102906 | 0.13102906 | GAIN | 0.666693496 | 0.79772256 | HOXA5; HOXA- |
| | | | | | | | | AS3 |
| cg17994139 | 4.25E−10 | 3.28466E−06 | 0.12790603 | 0.12790603 | GAIN | 0.038604284 | 0.166510311 | HOXA6; HOXA6; HOXA- |
| | | | | | | | | AS3; HOXA- |
| | | | | | | | | AS3 |
| cg14014955 | 1.02E−11 | 1.58003E−07 | 0.12376528 | 0.12376528 | GAIN | 0.758782409 | 0.882547693 | HOXA5; HOXA- |
| | | | | | | | | AS3 |
| cg24168308 | 1.13E−12 | 5.42167E−08 | 0.12357484 | 0.12357484 | GAIN | 0.616836109 | 0.740410953 | APP; APP; APP; APP; |
| | | | | | | | | APP; APP; APP; AP |
| cg00048370 | 8.98E−08 | 0.000268032 | 0.11921982 | 0.11921982 | GAIN | 0.598960227 | 0.718180047 | |
| cg00969405 | 1.69E−12 | 6.00554E−08 | 0.11655506 | 0.11655506 | GAIN | 0.756021891 | 0.872576947 | HOXA5; HOXA- |
| | | | | | | | | AS3 |
| cg23054456 | 1.63E−08 | 6.64247E−05 | 0.11540171 | 0.11540171 | GAIN | 0.56865384 | 0.684055553 | |
| cg23204968 | 2.52E−12 | 6.41032E−08 | 0.11131566 | 0.11131566 | GAIN | 0.808302327 | 0.919617987 | HOXA5; HOXA- |
| | | | | | | | | AS3 |
| cg08319974 | 4.07E−07 | 0.000786218 | 0.11088457 | 0.11088457 | GAIN | 0.525400382 | 0.636284953 | |
| cg22469274 | 5.31E−10 | 3.82878E−06 | 0.1106167 | 0.1106167 | GAIN | 0.044194163 | 0.154810859 | HOXA6; HOXA6; HOXA- |
| | | | | | | | | AS3; HOXA- |
| | | | | | | | | AS3 |
| cg15571561 | 3.57E−07 | 0.000725693 | 0.10922618 | 0.10922618 | GAIN | 0.188979984 | 0.298206167 | ARPP21; ARPP21; |
| cg16923485 | 2.74E−09 | 1.56011E−05 | 0.1078056 | 0.1078056 | GAIN | 0.460578478 | 0.568384073 | SLCO1A2; SLCO1A2 |
| cg19816811 | 6.01E−06 | 0.005092068 | 0.10705528 | 0.10705528 | GAIN | 0.519283504 | 0.626338787 | HOXA6; HOXA- |
| | | | | | | | | AS3; HOXA-AS3 |
| cg27151303 | 2.24E−06 | 0.002593592 | 0.10702693 | 0.10702693 | GAIN | 0.52929966 | 0.636326587 | HOXA-AS3 |
| cg24378559 | 4.81E−09 | 2.50546E−05 | 0.1052376 | 0.1052376 | GAIN | 0.442865982 | 0.54810358 | |
| cg20817131 | 2.52E−12 | 6.41032E−08 | 0.10463113 | 0.10463113 | GAIN | 0.7788633 | 0.883494433 | HOXA5; HOXA- |
| | | | | | | | | AS3 |
| cg25267863 | 1.04E−07 | 0.000297464 | 0.10446312 | 0.10446312 | GAIN | 0.292180311 | 0.396643427 | |
| cg05928186 | 2.56E−08 | 0.000102521 | 0.10372241 | 0.10372241 | GAIN | 0.44578503 | 0.54950744 | HOXA6; HOXA- |
| | | | | | | | | AS3; HOXA-AS3 |
| cg14658493 | 5.23E−12 | 9.42015E−08 | 0.10369845 | 0.10369845 | GAIN | 0.814620413 | 0.918318867 | HOXA5; HOXA- |
| | | | | | | | | AS3 |
| cg06388363 | 1.4E−06 | 0.001875868 | 0.10218302 | 0.10218302 | GAIN | 0.417598078 | 0.5197811 | |
| cg15297220 | 5.76E−08 | 0.000193245 | 0.10154844 | 0.10154844 | GAIN | 0.360729758 | 0.462278193 | |
| cg20974609 | 2.57E−11 | 3.27214E−07 | 0.10138775 | 0.10138775 | GAIN | 0.843193793 | 0.94458154 | HOXA5; HOXA- |
| | | | | | | | | AS3 |
| cg07070348 | 8.68E−07 | 0.001345676 | 0.10103344 | 0.10103344 | GAIN | 0.46125528 | 0.56228872 | |
| cg25174844 | 1.58E−06 | 0.002040974 | 0.10081944 | 0.10081944 | GAIN | 0.501924336 | 0.60274378 | |
| cg11096515 | 1.38E−07 | 0.00036862 | −0.10008737 | 0.10008737 | LOSS | 0.489812578 | 0.389725207 | COL4A2 |
| cg00026909 | 4.07E−07 | 0.000786218 | −0.10059218 | 0.10059218 | LOSS | 0.346087567 | 0.245495387 | DAB1 |
| cg20292791 | 1.11E−06 | 0.001570483 | −0.10069147 | 0.10069147 | LOSS | 0.805879264 | 0.705187793 | DAB1 |
| cg23772122 | 2.74E−07 | 0.000596022 | −0.10145315 | 0.10145315 | LOSS | 0.61480136 | 0.513348213 | ANO3 |
| cg24796998 | 6.69E−08 | 0.000217703 | −0.10167643 | 0.10167643 | LOSS | 0.499954038 | 0.398277607 | |
| cg24750308 | 1.2E−07 | 0.000334393 | −0.1019464 | 0.1019464 | LOSS | 0.487743396 | 0.385796993 | NOX4; NOX4; NOX4; |
| | | | | | | | | NOX4 |
| cg21758126 | 1.12E−05 | 0.007790401 | −0.1025113 | 0.1025113 | LOSS | 0.538915104 | 0.4364038 | NR4A2 |
| cg07769947 | 8.98E−08 | 0.000268032 | −0.10260998 | 0.10260998 | LOSS | 0.546605162 | 0.443995187 | |
| cg20955836 | 8.23E−06 | 0.006348011 | −0.10270363 | 0.10270363 | LOSS | 0.339719751 | 0.23701612 | BMP7 |
| cg14897238 | 6.01E−06 | 0.005092068 | −0.10271335 | 0.10271335 | LOSS | 0.434702618 | 0.331989267 | |
| cg01450725 | 4.25E−08 | 0.000155777 | −0.10307694 | 0.10307694 | LOSS | 0.340429131 | 0.237352193 | |
| cg09113483 | 9.12E−06 | 0.006794034 | −0.10330673 | 0.10330673 | LOSS | 0.664091156 | 0.560784427 | |
| cg22011526 | 1.37E−05 | 0.008679024 | −0.10340502 | 0.10340502 | LOSS | 0.747030156 | 0.643625133 | C6orf89; C6orf89; |
| | | | | | | | | C6orf89; C6orf89 |
| cg23900293 | 1.11E−06 | 0.001570483 | −0.10436904 | 0.10436904 | LOSS | 0.528886009 | 0.424516973 | |
| cg09741912 | 8.23E−09 | 3.82658E−05 | −0.10475007 | 0.10475007 | LOSS | 0.768298233 | 0.663548167 | |
| cg11598935 | 9.81E−07 | 0.001437937 | −0.10498738 | 0.10498738 | LOSS | 0.516474982 | 0.411487607 | BMP7 |
| cg11704490 | 7.42E−06 | 0.005879569 | −0.10499976 | 0.10499976 | LOSS | 0.707848818 | 0.602849053 | |
| cg19655952 | 1.24E−09 | 8.13644E−06 | −0.10503044 | 0.10503044 | LOSS | 0.652592453 | 0.547562013 | FOXP2; FOXP2; FOXP2; |
| | | | | | | | | FOXP2; FOXP |
| cg15801019 | 2.8E−06 | 0.003048286 | −0.10518774 | 0.10518774 | LOSS | 0.511141729 | 0.405953987 | LAMA2; LAMA2 |
| cg20811236 | 1.12E−05 | 0.007790401 | −0.10538143 | 0.10538143 | LOSS | 0.52218622 | 0.416804793 | |
| cg16968885 | 8.68E−07 | 0.001345676 | −0.10543721 | 0.10543721 | LOSS | 0.785359396 | 0.679922187 | COL11A1; COL11A1; |
| | | | | | | | | COL11A1; COL1 |
| cg20592075 | 1.2E−07 | 0.000334393 | −0.10571857 | 0.10571857 | LOSS | 0.644023313 | 0.538304747 | |
| cg19743254 | 1.01E−05 | 0.007274077 | −0.10610715 | 0.10610715 | LOSS | 0.625013382 | 0.518906233 | OPCML; OPCML |
| cg27536286 | 6.78E−07 | 0.001158528 | −0.10668088 | 0.10668088 | LOSS | 0.651200002 | 0.544519127 | |
| cg25436634 | 6.78E−07 | 0.001158528 | −0.10737378 | 0.10737378 | LOSS | 0.523050418 | 0.415676633 | SOX2-OT; SOX2- |
| | | | | | | | | OT; SOX2-OT |
| cg18951332 | 1.67E−10 | 1.5341E−06 | −0.10767814 | 0.10767814 | LOSS | 0.738900909 | 0.631222767 | |
| cg24526899 | 1.37E−05 | 0.008679024 | −0.10776459 | 0.10776459 | LOSS | 0.669045324 | 0.561280733 | BMP4; BMP4 |
| cg22321572 | 1.25E−06 | 0.001702533 | −0.10825609 | 0.10825609 | LOSS | 0.317658142 | 0.209402057 | MLLT4-AS1 |
| cg10228555 | 5.41E−06 | 0.004745665 | −0.10858265 | 0.10858265 | LOSS | 0.453158811 | 0.34457616 | LOC100128770 |
| cg25008182 | 3.32E−09 | 1.77048E−05 | −0.10958067 | 0.10958067 | LOSS | 0.834894593 | 0.725313927 | |
| cg20263045 | 2.24E−06 | 0.002593592 | −0.10962959 | 0.10962959 | LOSS | 0.767880947 | 0.658251353 | HHIP |
| cg06602723 | 1.52E−09 | 9.53506E−06 | −0.10984647 | 0.10984647 | LOSS | 0.377775487 | 0.26792902 | HOXB8 |
| cg25701444 | 5.26E−07 | 0.000952952 | −0.10987376 | 0.10987376 | LOSS | 0.733034304 | 0.62316054 | LOC400043 |
| cg10886095 | 4.36E−06 | 0.004146153 | −0.11155878 | 0.11155878 | LOSS | 0.557715016 | 0.446156233 | CCDC60 |
| cg13749822 | 1.66E−05 | 0.009675336 | −0.11160844 | 0.11160844 | LOSS | 0.300161093 | 0.188552656 | HHIP; HHIP-AS1 |
| cg17654050 | 1.58E−06 | 0.002040974 | −0.11169961 | 0.11169961 | LOSS | 0.54445336 | 0.432753753 | NR4A2 |
| cg26673377 | 3.57E−07 | 0.000725693 | −0.11233179 | 0.11233179 | LOSS | 0.683894816 | 0.571563027 | |
| cg08657492 | 1.51E−05 | 0.00917751 | −0.11328691 | 0.11328691 | LOSS | 0.51272948 | 0.399442573 | HOXA4 |
| cg20706134 | 2.09E−07 | 0.000494656 | −0.11378449 | 0.11378449 | LOSS | 0.722188707 | 0.60840422 | PCDH20 |
| cg05232889 | 1.16E−08 | 4.88234E−05 | −0.11419818 | 0.11419818 | LOSS | 0.758496844 | 0.64429866 | FOXP2; FOXP2; FOXP2; |
| | | | | | | | | FOXP2; FOXP |
| cg07659054 | 8.23E−09 | 3.82658E−05 | −0.11443317 | 0.11443317 | LOSS | 0.361695007 | 0.24726184 | HOXA1; HOXA1; HOTAIRM1; |
| | | | | | | | | HOTAIRM1 |
| cg12806882 | 4.25E−08 | 0.000155777 | −0.11453851 | 0.11453851 | LOSS | 0.566396433 | 0.45185792 | FMN2 |
| cg18871253 | 2.74E−09 | 1.56011E−05 | −0.11623746 | 0.11623746 | LOSS | 0.736403029 | 0.620165567 | FOXP2; FOXP2; FOXP2; |
| | | | | | | | | FOXP2; FOXP |
| cg25942940 | 1.4E−11 | 1.95705E−07 | −0.1171622 | 0.1171622 | LOSS | 0.781304316 | 0.664142113 | |
| cg13320964 | 9.81E−07 | 0.001437937 | −0.11776404 | 0.11776404 | LOSS | 0.661345138 | 0.543581093 | |
| cg17461600 | 2.74E−07 | 0.000596022 | −0.11792734 | 0.11792734 | LOSS | 0.691368147 | 0.573440807 | DAB1 |
| cg15648345 | 3.13E−07 | 0.000654544 | −0.1181466 | 0.1181466 | LOSS | 0.613033044 | 0.494886447 | MKS1; MKS1 |
| cg18546840 | 1.86E−09 | 1.14743E−05 | −0.11855055 | 0.11855055 | LOSS | 0.790301131 | 0.67175058 | FOXP2; FOXP2; FOXP2; |
| | | | | | | | | FOXP2; FOXP |
| cg09203312 | 1.4E−06 | 0.001875868 | −0.12039032 | 0.12039032 | LOSS | 0.671637149 | 0.551246833 | GJB6; GJB6; GJB6; |
| | | | | | | | | GJB6; GJB6 |
| cg02211646 | 1.3E−10 | 1.25096E−06 | −0.12069127 | 0.12069127 | LOSS | 0.779895987 | 0.65920472 | FOXP2; FOXP2; FOXP2; |
| | | | | | | | | FOXP2; FOXP |
| cg18805066 | 9.79E−09 | 4.36653E−05 | −0.12069718 | 0.12069718 | LOSS | 0.287220951 | 0.166523775 | HOXA1; HOXA1; HOTAIRM1; |
| | | | | | | | | HOTAIRM1 |
| cg00428457 | 5.41E−06 | 0.004745665 | −0.12072828 | 0.12072828 | LOSS | 0.769902418 | 0.64917414 | |
| cg01746241 | 1.11E−06 | 0.001570483 | −0.12217225 | 0.12217225 | LOSS | 0.676761909 | 0.55458966 | KIAA1161 |
| cg24549912 | 5.26E−07 | 0.000952952 | −0.12223264 | 0.12223264 | LOSS | 0.336860567 | 0.214627927 | |
| cg11857140 | 3.32E−09 | 1.77048E−05 | −0.1236919 | 0.1236919 | LOSS | 0.735227656 | 0.61153576 | KIRREL3; KIRREL3 |
| cg24786986 | 5.31E−10 | 3.82878E−06 | −0.12397933 | 0.12397933 | LOSS | 0.7449648 | 0.620985473 | FOXP2; FOXP2; FOXP2; |
| | | | | | | | | FOXP2; FOXP |
| cg08959039 | 5.41E−06 | 0.004745665 | −0.12404245 | 0.12404245 | LOSS | 0.472527078 | 0.348484627 | COL4A2 |
| cg22154659 | 2.69E−10 | 2.32622E−06 | −0.12878474 | 0.12878474 | LOSS | 0.537517758 | 0.408733013 | HOXA1; HOXA1; HOTAIRM1; |
| | | | | | | | | HOTAIRM1 |
| cg09517766 | 9.81E−07 | 0.001437937 | −0.12995329 | 0.12995329 | LOSS | 0.6572662 | 0.527312913 | |
| cg15161959 | 9.79E−09 | 4.36653E−05 | −0.13054628 | 0.13054628 | LOSS | 0.477714369 | 0.347168087 | |
| cg11758841 | 7.79E−11 | 8.84149E−07 | −0.13651604 | 0.13651604 | LOSS | 0.709946164 | 0.573430127 | PARVA |
| cg25598685 | 1.16E−08 | 4.88234E−05 | −0.13769051 | 0.13769051 | LOSS | 0.73225986 | 0.594569353 | |
| cg25556579 | 1.01E−09 | 6.93405E−06 | −0.13814846 | 0.13814846 | LOSS | 0.509734767 | 0.371586307 | TBX5; TBX5; TBX5 |
| cg25037165 | 6.61E−10 | 4.68485E−06 | −0.13905994 | 0.13905994 | LOSS | 0.884593136 | 0.745533193 | TEAD1 |
| cg06218338 | 3.91E−06 | 0.003844825 | −0.13951377 | 0.13951377 | LOSS | 0.290344878 | 0.150831105 | |
| cg26264232 | 1.16E−08 | 4.88234E−05 | −0.13993212 | 0.13993212 | LOSS | 0.272384147 | 0.132452026 | HOTAIRM1; HOTAIRM1 |
| cg19981409 | 9.79E−09 | 4.36653E−05 | −0.14255474 | 0.14255474 | LOSS | 0.442544042 | 0.299989307 | NOX4; NOX4; NOX4; |
| | | | | | | | | NOX4 |
| cg23111488 | 1.01E−09 | 6.93405E−06 | −0.1498176 | 0.1498176 | LOSS | 0.759792356 | 0.609974753 | |
| cg00525681 | 3.91E−06 | 0.003844825 | −0.14995196 | 0.14995196 | LOSS | 0.615382438 | 0.465430473 | SLITRK5 |
| cg06911613 | 4.95E−08 | 0.000174192 | −0.15045288 | 0.15045288 | LOSS | 0.649693296 | 0.49924042 | |
| cg06906435 | 1.24E−09 | 8.13644E−06 | −0.15057591 | 0.15057591 | LOSS | 0.527015647 | 0.376439733 | C14orf177 |
| cg17376609 | 1.59E−07 | 0.000404111 | −0.15256603 | 0.15256603 | LOSS | 0.752634513 | 0.60006848 | SLITRK5 |
| cg13746854 | 2.37E−06 | 0.002735892 | −0.15289842 | 0.15289842 | LOSS | 0.53473938 | 0.38184096 | KIAA1161 |
| cg12115302 | 1.38E−07 | 0.00036862 | −0.15317369 | 0.15317369 | LOSS | 0.490363404 | 0.337189717 | |
| cg08657654 | 1.59E−07 | 0.000404111 | −0.1542548 | 0.1542548 | LOSS | 0.769303353 | 0.615048553 | HOTAIRM1; HOTAIRM1 |
| cg16370398 | 1.66E−06 | 0.002126946 | −0.15506739 | 0.15506739 | LOSS | 0.511144073 | 0.35607668 | HOXC4; HOXC4 |
| cg16787483 | 4.07E−07 | 0.000786218 | −0.16228425 | 0.16228425 | LOSS | 0.717952758 | 0.555668507 | SLITRK5 |
| cg21090457 | 3.39E−10 | 2.76566E−06 | −0.16579188 | 0.16579188 | LOSS | 0.617719138 | 0.451927253 | ROBO2; ROBO2 |
| cg16915863 | 3.14E−06 | 0.003366444 | −0.16787453 | 0.16787453 | LOSS | 0.773898147 | 0.606023613 | LOC400043 |
| cg08941355 | 5.31E−10 | 3.82878E−06 | −0.17084311 | 0.17084311 | LOSS | 0.672878658 | 0.502035547 | HOXA1; HOXA1 |
| cg03906434 | 4.36E−06 | 0.004146153 | −0.17673643 | 0.17673643 | LOSS | 0.316082895 | 0.13934647 | |
| cg09823859 | 5.76E−08 | 0.000193245 | −0.17889435 | 0.17889435 | LOSS | 0.654204142 | 0.475309793 | SLITRK5 |
| cg05757365 | 4.07E−07 | 0.000786218 | −0.17921325 | 0.17921325 | LOSS | 0.614664191 | 0.43545094 | SLITRK5 |
| cg04707013 | 6.01E−06 | 0.005092068 | −0.18328041 | 0.18328041 | LOSS | 0.707909664 | 0.52462925 | |
| cg23865240 | 7.33E−12 | 1.21988E−07 | −0.18696432 | 0.18696432 | LOSS | 0.505623411 | 0.318659087 | HOXA1; HOXA1 |
| cg18751141 | 7.97E−11 | 8.84149E−07 | −0.1901929 | 0.1901929 | LOSS | 0.497364882 | 0.30717198 | HOTAIRM1; HOTAIRM1 |
| cg24626752 | 8.68E−07 | 0.001345676 | −0.19303999 | 0.19303999 | LOSS | 0.676344478 | 0.483304487 | SLITRK5 |
| cg17881200 | 2.69E−10 | 2.32622E−06 | −0.19311652 | 0.19311652 | LOSS | 0.507025549 | 0.313909033 | HOTAIRM1; HOTAIRM1 |
| cg26168643 | 1.78E−06 | 0.002207935 | −0.19328866 | 0.19328866 | LOSS | 0.608304222 | 0.41501556 | SLITRK5 |
| cg17485838 | 3.39E−10 | 2.76566E−06 | −0.19332356 | 0.19332356 | LOSS | 0.540119302 | 0.34679574 | HOTAIRM1; HOTAIRM1 |
| cg02611934 | 1.82E−07 | 0.000448384 | −0.2035436 | 0.2035436 | LOSS | 0.608860269 | 0.405316673 | SLITRK5 |
| cg07278425 | 5.23E−12 | 9.42015E−08 | −0.21052294 | 0.21052294 | LOSS | 0.616841918 | 0.40631898 | HOTAIRM1; HOTAIRM1 |
| cg07318204 | 1.38E−07 | 0.00036862 | −0.21626875 | 0.21626875 | LOSS | 0.744438569 | 0.52816982 | HHIP; HHIP-AS1 |
| cg00106345 | 5.98E−11 | 6.98956E−07 | −0.219793 | 0.219793 | LOSS | 0.455452644 | 0.235659647 | HOTAIRM1; HOTAIRM1 |

| | | | Genomic | | | Relation to |
|---|---|---|---|---|---|---|
| | | | Coordinate | | | transcription |
| Illumine ID | Genome_Build | Chromosome | (NCBI, hg19) | Strand | Relation_to_UCSC_CpG_Island | start site (TSS) |
| cg17569124 | 37 | 7 | 27183643 | − | Island | HOXA- |
| | | | | | | AS3(body); HOXA5 |
| | | | | | | (tss1500) |
| cg25307665 | 37 | 7 | 27183694 | − | Island | HOXA- |
| | | | | | | AS3(body); HOXA5 |
| | | | | | | (tss1500) |
| cg12128839 | 37 | 7 | 27183436 | − | Island | HOXA- |
| | | | | | | AS3(body); HOXA5 |
| | | | | | | (tss200) |
| cg05076221 | 37 | 7 | 27182637 | + | Island | HOXA- |
| | | | | | | AS3(body); HOXA5 |
| | | | | | | (body) |
| cg19759481 | 37 | 7 | 27183401 | − | Island | HOXA- |
| | | | | | | AS3(body); HOXA5 |
| | | | | | | (tss200) |
| cg04863892 | 37 | 7 | 27183375 | − | Island | HOXA- |
| | | | | | | AS3(body); HOXA5 |
| | | | | | | (tss200) |
| cg04053108 | 37 | 12 | 6166028 | − | Island | VWF(body) |
| cg02005600 | 37 | 7 | 27183686 | − | Island | HOXA- |
| | | | | | | AS3(body); HOXA5 |
| | | | | | | (tss1500) |
| cg23936031 | 37 | 7 | 27183133 | + | Island | HOXA- |
| | | | | | | AS3(body); HOXA5 |
| | | | | | | (body) |
| cg09319828 | 37 | 1 | 156551787 | − | | TTC24(body) |
| cg02916332 | 37 | 7 | 27183591 | + | Island | HOXA- |
| | | | | | | AS3(body); HOXA5 |
| | | | | | | (tss1500) |
| cg03368099 | 37 | 7 | 27184521 | − | Island | HOXA- |
| | | | | | | AS3(body); HOXA5 |
| | | | | | | (tss1500) |
| cg11724970 | 37 | 7 | 27182493 | − | N_Shore | HOXA- |
| | | | | | | AS3(body); HOXA5 |
| | | | | | | (body) |
| cg18274664 | 37 | 21 | 27372461 | − | | APP(body) |
| cg03529432 | 37 | 7 | 27187502 | − | Island | HOXA- |
| | | | | | | AS3(body); HOXA6 |
| | | | | | | (tss200) |
| cg05835726 | 37 | 7 | 27183861 | − | Island | HOXA- |
| | | | | | | AS3(body); HOXA5 |
| | | | | | | (tss1500) |
| cg02248486 | 37 | 7 | 27183196 | − | Island | HOXA- |
| | | | | | | AS3(body); HOXA5 |
| | | | | | | (body) |
| cg17432857 | 37 | 7 | 27184438 | − | Island | HOXA- |
| | | | | | | AS3(body); HOXA5 |
| | | | | | | (tss1500) |
| cg14882265 | 37 | 7 | 27184375 | + | Island | HOXA- |
| | | | | | | AS3(body); HOXA5 |
| | | | | | | (tss1500) |
| cg11321156 | 37 | 21 | 27372396 | − | | APP(body) |
| cg01370449 | 37 | 7 | 27183369 | + | Island | HOXA- |
| | | | | | | AS3(body); HOXA5 |
| | | | | | | (tss200) |
| cg20517050 | 37 | 7 | 27183806 | − | Island | HOXA- |
| | | | | | | AS3(body); HOXA5 |
| | | | | | | (tss1500) |
| cg14044640 | 37 | 7 | 27187560 | + | Island | HOXA- |
| | | | | | | AS3(body); HOXA6 |
| | | | | | | (tss200) |
| cg23269692 | 37 | 21 | 27372446 | + | | APP(body) |
| cg23129930 | 37 | 7 | 27186993 | + | Island | HOXA- |
| | | | | | | AS3(body); HOXA6 |
| | | | | | | (body) |
| cg26023912 | 37 | 7 | 27184369 | + | Island | HOXA- |
| | | | | | | AS3(body); HOXA5 |
| | | | | | | (tss1500) |
| cg25866143 | 37 | 7 | 27183262 | + | Island | HOXA- |
| | | | | | | AS3(body); HOXA5 |
| | | | | | | (body) |
| cg06237983 | 37 | 7 | 27187269 | + | Island | HOXA- |
| | | | | | | AS3(body); HOXA6 |
| | | | | | | (body) |
| cg02646423 | 37 | 7 | 27183794 | − | Island | HOXA- |
| | | | | | | AS3(body); HOXA5 |
| | | | | | | (tss1500) |
| cg17994139 | 37 | 7 | 27187556 | + | Island | HOXA- |
| | | | | | | AS3(body); HOXA6 |
| | | | | | | (tss200) |
| cg14014955 | 37 | 7 | 27183701 | + | Island | HOXA- |
| | | | | | | AS3(body); HOXA5 |
| | | | | | | (tss1500) |
| cg24168308 | 37 | 21 | 27372387 | − | | APP(body) |
| cg00048370 | 37 | 6 | 164506939 | − | | |
| cg00969405 | 37 | 7 | 27184441 | − | Island | HOXA- |
| | | | | | | AS3(body); HOXA5 |
| | | | | | | (tss1500) |
| cg23054456 | 37 | 6 | 164506981 | − | | |
| cg23204968 | 37 | 7 | 27183816 | − | Island | HOXA- |
| | | | | | | AS3(body); HOXA5 |
| | | | | | | (tss1500) |
| cg08319974 | 37 | 6 | 164506861 | + | | |
| cg22469274 | 37 | 7 | 27187553 | + | Island | HOXA- |
| | | | | | | AS3(body); HOXA6 |
| | | | | | | (tss200) |
| cg15571561 | 37 | 3 | 35706161 | − | | ARPP21(body) |
| cg16923485 | 37 | 12 | 21476904 | + | | SLCO1A2(body) |
| cg19816811 | 37 | 7 | 27188364 | + | N_Shore | HOXA- |
| | | | | | | AS3(body); HOXA6 |
| | | | | | | (tss1500) |
| cg27151303 | 37 | 7 | 27184821 | − | Island | HOXA- |
| | | | | | | AS3(body) |
| cg24378559 | 37 | 7 | 156889254 | + | | |
| cg20817131 | 37 | 7 | 27184167 | − | Island | HOXA- |
| | | | | | | AS3(body); HOXA5 |
| | | | | | | (tss1500) |
| cg25267863 | 37 | 7 | 1363124 | − | Island | |
| cg05928186 | 37 | 7 | 27187102 | + | Island | HOXA- |
| | | | | | | AS3(body); HOXA6 |
| | | | | | | (body) |
| cg14658493 | 37 | 7 | 27184077 | − | Island | HOXA- |
| | | | | | | AS3(body); HOXA5 |
| | | | | | | (tss1500) |
| cg06388363 | 37 | 6 | 164507305 | − | | |
| cg15297220 | 37 | 4 | 134589655 | + | | |
| cg20974609 | 37 | 7 | 27181671 | − | N_Shore | HOXA- |
| | | | | | | AS3(body); HOXA5 |
| | | | | | | (body) |
| cg07070348 | 37 | 12 | 130555007 | + | | |
| cg25174844 | 37 | 15 | 73195113 | + | | |
| cg11096515 | 37 | 13 | 111062287 | + | | COL4A2(body) |
| cg00026909 | 37 | 1 | 58089001 | + | | DAB1(body) |
| cg20292791 | 37 | 1 | 58089357 | + | | DAB1(body) |
| cg23772122 | 37 | 11 | 26355628 | + | S_Shore | ANO3(body) |
| cg24796998 | 37 | 17 | 70383845 | + | | |
| cg24750308 | 37 | 11 | 89225014 | − | S_Shore | NOX4(body); NOX4 |
| | | | | | | (tss1500) |
| cg21758126 | 37 | 2 | 157183291 | − | N_Shore | NR4A2(body) |
| cg07769947 | 37 | 2 | 220601262 | − | | |
| cg20955836 | 37 | 20 | 55836224 | + | N_Shelf | BMP7(body) |
| cg14897238 | 37 | 21 | 43198283 | − | N_Shore | |
| cg01450725 | 37 | 4 | 154714852 | + | S_Shore | |
| cg09113483 | 37 | 1 | 61517807 | − | N_Shore | |
| cg22011526 | 37 | 6 | 36857605 | + | S_Shelf | C6orf89(body) |
| cg23900293 | 37 | 11 | 115924505 | − | | |
| cg09741912 | 37 | 11 | 114921894 | − | | |
| cg11598935 | 37 | 20 | 55837619 | + | N_Shore | BMP7(body) |
| cg11704490 | 37 | 2 | 162284894 | − | S_Shore | |
| cg19655952 | 37 | 7 | 114055204 | + | | FOXP2(body) |
| cg15801019 | 37 | 6 | 129203783 | − | | LAMA2(tss1500) |
| cg20811236 | 37 | 18 | 46501400 | + | N_Shore | |
| cg16968885 | 37 | 1 | 103574619 | − | | COL11A1(tss1500) |
| cg20592075 | 37 | 7 | 45921668 | − | | |
| cg19743254 | 37 | 11 | 132735814 | + | | OPCML(body) |
| cg27536286 | 37 | 13 | 27414220 | + | | |
| cg25436634 | 37 | 3 | 181045270 | + | | SOX2-OT(body) |
| cg18951332 | 37 | 2 | 220777552 | − | | |
| cg24526899 | 37 | 14 | 54424149 | + | S_Shore | BMP4(tss1500) |
| cg22321572 | 37 | 6 | 168225923 | − | N_Shore | MLLT4- |
| | | | | | | AS1(body) |
| cg10228555 | 37 | 16 | 3088480 | + | S_Shore | LOC100128770 |
| | | | | | | (body) |
| cg25008182 | 37 | 3 | 182123703 | + | | |
| cg20263045 | 37 | 4 | 145655974 | − | | HHIP(body) |
| cg06602723 | 37 | 17 | 46693336 | − | N_Shore | HOXB8(tss1500) |
| cg25701444 | 37 | 12 | 54521977 | − | S_Shore | LOC400043(body) |
| cg10886095 | 37 | 12 | 119935697 | − | | CCDC60(body) |
| cg13749822 | 37 | 4 | 145566663 | − | Island | HHIP(tss1500); |
| | | | | | | HHIP- |
| | | | | | | AS1(body) |
| cg17654050 | 37 | 2 | 157184978 | + | N_Shore | NR4A2(body) |
| cg26673377 | 37 | 6 | 123182996 | + | | |
| cg08657492 | 37 | 7 | 27170832 | + | S_Shore | HOXA4(tss1500) |
| cg20706134 | 37 | 13 | 61990025 | + | | PCDH20(tss1500) |
| cg05232889 | 37 | 7 | 114055419 | + | | FOXP2(body) |
| cg07659054 | 37 | 7 | 27134225 | − | Island | HOTAIRM1(tss1500); |
| | | | | | | HOXA1(body) |
| cg12806882 | 37 | 1 | 240572391 | − | S_Shelf | FMN2(body) |
| cg18871253 | 37 | 7 | 114055137 | + | | FOXP2(body) |
| cg25942940 | 37 | 1 | 8270645 | − | N_Shore | |
| cg13320964 | 37 | 4 | 138114823 | + | | |
| cg17461600 | 37 | 1 | 57983368 | − | | DAB1(body) |
| cg15648345 | 37 | 17 | 56297360 | + | S_Shore | MKS1(tss1500) |
| cg18546840 | 37 | 7 | 114055123 | + | | FOXP2(body) |
| cg09203312 | 37 | 13 | 20805196 | + | N_Shore | GJB6(body) |
| cg02211646 | 37 | 7 | 114055210 | + | | FOXP2(body) |
| cg18805066 | 37 | 7 | 27134259 | − | Island | HOTAIRM1(tss1500); |
| | | | | | | HOXA1(body) |
| cg00428457 | 37 | 2 | 119887680 | − | | |
| cg01746241 | 37 | 9 | 34370835 | − | Island | KIAA1161(body) |
| cg24549912 | 37 | 5 | 50692281 | + | N_Shelf | |
| cg11857140 | 37 | 11 | 126372533 | + | | KIRREL3(body) |
| cg24786986 | 37 | 7 | 114055133 | + | | FOXP2(body) |
| cg08959039 | 37 | 13 | 111062266 | + | | COL4A2(body) |
| cg22154659 | 37 | 7 | 27134369 | − | N_Shore | HOTAIRM1(tss1500); |
| | | | | | | HOXA1(body) |
| cg09517766 | 37 | 10 | 44894102 | − | | |
| cg15161959 | 37 | 2 | 177020616 | − | N_Shelf | |
| cg11758841 | 37 | 11 | 12530155 | + | | PARVA(body) |
| cg25598685 | 37 | 11 | 42617544 | + | | |
| cg25556579 | 37 | 12 | 114829194 | + | | TBX5(body) |
| cg25037165 | 37 | 11 | 12824283 | − | | TEAD1(body) |
| cg06218338 | 37 | 7 | 27231894 | − | Island | |
| cg26264232 | 37 | 7 | 27138751 | − | S_Shelf | HOTAIRM1(body) |
| cg19981409 | 37 | 11 | 89225042 | − | S_Shore | NOX4(body); NOX4 |
| | | | | | | (tss1500) |
| cg23111488 | 37 | 5 | 144538350 | − | | |
| cg00525681 | 37 | 13 | 88329151 | − | N_Shore | SLITRK5(body) |
| cg06911613 | 37 | 16 | 85846184 | − | S_Shore | |
| cg06906435 | 37 | 14 | 99177777 | − | | C14orf177(tss200) |
| cg17376609 | 37 | 13 | 88328813 | + | N_Shore | SLITRK5(body) |
| cg13746854 | 37 | 9 | 34370894 | − | Island | KIAA1161(body) |
| cg12115302 | 37 | 12 | 30323676 | + | S_Shore | |
| cg08657654 | 37 | 7 | 27138974 | + | S_Shelf | HOTAIRM1(body) |
| cg16370398 | 37 | 12 | 54448913 | + | S_Shore | HOXC4(body) |
| cg16787483 | 37 | 13 | 88328251 | − | N_Shore | SLITRK5(body) |
| cg21090457 | 37 | 3 | 77573709 | + | | ROBO2(body) |
| cg16915863 | 37 | 12 | 54523294 | + | S_Shelf | LOC400043(body) |
| cg08941355 | 37 | 7 | 27133106 | − | N_Shore | HOXA1(body) |
| cg03906434 | 37 | 7 | 27231819 | − | Island | |
| cg09823859 | 37 | 13 | 88328294 | + | N_Shore | SLITRK5(body) |
| cg05757365 | 37 | 13 | 88328471 | + | N_Shore | SLITRK5(body) |
| cg04707013 | 37 | 10 | 111177826 | − | | |
| cg23865240 | 37 | 7 | 27134109 | + | Island | HOXA1(body) |
| cg18751141 | 37 | 7 | 27138173 | + | S_Shore | HOTAIRM1(body) |
| cg24626752 | 37 | 13 | 88328274 | + | N_Shore | SLITRK5(body) |
| cg17881200 | 37 | 7 | 27138850 | + | S_Shelf | HOTAIRM1(body) |
| cg26168643 | 37 | 13 | 88328009 | − | N_Shore | SLITRK5(body) |
| cg17485838 | 37 | 7 | 27138712 | − | S_Shore | HOTAIRM1(body) |
| cg02611934 | 37 | 13 | 88329407 | + | Island | SLITRK5(body) |
| cg07278425 | 37 | 7 | 27137922 | + | S_Shore | HOTAIRM1(body) |
| cg07318204 | 37 | 4 | 145566441 | − | Island | HHIP(tss1500); |
| | | | | | | HHIP- |
| | | | | | | AS1(body) |
| cg00106345 | 37 | 7 | 27138396 | + | S_Shore | HOTAIRM1(body) |

“Mean not-CHARGE” refers to the mean β-value for the CpG loci in the non-CS cases.
“Mean CHARGE” refers to the mean β-value for the CpG loci in the CS samples.

TABLE 3
Cross-validation results for different effect-size (absolute delta beta, |Δβ|)
thresholds at p-value <0.01. Shown are the specificity (Spec) and sensitivity
(Sens) of the LOO procedure, specificity for 1056 normal blood samples derived
from GEO (Spec GEO). The total number of significant sites (CGs) in the resulting
“CHD7 signature” set, the gene names (Names) and their total number (Genes)
corresponding to the significant sites. One optimal combination (highlighted in bold)
was selected to be p-value <0.01 and |Δβ| >10%. The p-values are
corrected for multiple testing (Benjamini-Hochberg correction).

| | p-value <0.01 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 99.9% | 542 | ACAP2; ADAMTS17; ADCY5; ADIRF; ADORA2B; ALDH1A3; ALX3; ANK1; | 224 |
| | | | | | ANO3; APP; ARHGEF15; ARHGEF4; ARPP21; ARSJ; ATXN7L1; AXIN2; | |
| | | | | | BMP4; BMP5; BMP7; BMPER; BRE; BRINP1; C10orf90; C11orf88; | |
| | | | | | C14orf177; C14orf64; C19orf45; C1orf53; C6orf89; CACNA1H; | |
| | | | | | CADM3; CAMTA1; CCDC60; CCSER1; CD226; CD9; CLMP; CMTM7; COL11A1; | |
| | | | | | COL21A1; COL4A2; COLEC12; DAB1; DAW1; DIP2C; DLC1; DMRT1; DMXL1; | |
| | | | | | DNER; DOK1; EBF3; ELAVL2; EMILIN2; EPAS1; EPB41L1; ERBB2; ERC2; | |
| | | | | | ERMN; EVI5; EVP1; FAM155A; FAM19A1; FAM83F; FCGRT; FGF2; FGF23; | |
| | | | | | FLJ12825; FLJ39080; FLOT1; FMN2; FOXK1; FOXP1; FOXP2; FRMD3; | |
| | | | | | GABBR1; GDF2; GIPC2; GJB6; GPATCH2; GPR151; GPRC5C; GRB7; GRID1; | |
| | | | | | GRID2; HECW1; HHIP; HHIP-AS1; HOTAIRM1; HOXA-AS3; HOXA1; HOXA10; | |
| | | | | | HOXA10-HOXA9; HOXA11; HOXA11-AS; HOXA2; HOXA4; HOXA5; HOXA6; | |
| | | | | | HOXB8; HOXC4; HOXC5; HOXC6; HOXD9; HTR5A; IGF2; IGF2-AS; IGFBP5; | |
| | | | | | IL20RA; INS-IGF2; ISG20; ISL1; KCNJ6; KCNQ4; KIAA0922; KIAA1161; | |
| | | | | | KIRREL3; KLHL14; L3MBTL4; LAMA2; LCE3A; LHX4; LINC00554; | |
| | | | | | LINC00601; LINC00982; LMO3; LOC100128239; LOC100128770; | |
| | | | | | LOC100996291; LOC145845; LOC400043; LOC400456; LOC642366; LRRC4C; | |
| | | | | | MAFA; MFSD1; MIR10B; MIR1284; MKS1; MLLT4-AS1; MOB2; MS4A6A; | |
| | | | | | MUC21; MYO1F; NCKAP5; NFIB; NKAIN3; NKX3-1; NOX4; NPSR1; NPSR1- | |
| | | | | | AS1; NR4A2; NRARP; NXN; OPCM1; OPRM1; PALM2; PALM2-AKAP2; PARVA; | |
| | | | | | PCDH15; PCDH20; PDE4C; PDZRN3; PGLYRP1; PKNOX2; PLBD1; PNLIPRP3; | |
| | | | | | POSTN; PRLR; PRMT8; PRSS56; PSAPL1; PTCHD4; PVRL3; PVRL3-AS1; | |
| | | | | | RAB3C; RAC1; RARRES2; RBFOX3; RELN; RGS17; RGS7; RNF180; ROBO1; | |
| | | | | | ROBO2; RUNX1T1; SEC24D; SGPP2; SHISA9; SLC1A3; SLC24A4; SLC35C1; | |
| | | | | | SLCO1A2; SLFN12; SLITRK5; SLPI; SORCS2; SOX2-OT; SOX7; SPATA17; | |
| | | | | | STEAP2; SYNE1; TBX3; TBX5; TEAD1; TENM4; TFAP2A; TMCC1; TMCC1-AS1; | |
| | | | | | TMEM132C; TPO; TRUB1; TSPAN4; TTC24; TUBGCP3; UGP2; VWF; WFDC2; | |
| | | | | | WNT7A; ZCCHC14; ZDHHC22; ZEB1; ZFP64; ZIC4; ZNF586 | |
| 10% | 100.0% | 100.0% | 99.5% | 146 | ANO3; APP; ARPP21; BMP4; BMP7; C14orf77; C6orf89; CCDC60; | 44 |
| | | | | | COL11A1; COL4A2; DAB1; FMN2; FOXP2; GJB6; HHIP; HHIP-AS1; | |
| | | | | | HOTAIRM1; HOXA-AS3; HOXA1; HOXA4; HOXA5; HOXA6; HOXB8; HOXC4; | |
| | | | | | KIAA1161; KIRREL3; LAMA2; LOC100128770; LOC400043; MKS1; | |
| | | | | | MLLT4-AS1; NOX4; NR4A2; OPCM1; PARVA; PCDH20; ROBO2; SLCO1A2; | |
| | | | | | SLITRK5; SOX2-OT; TBX5; TEAD1; TTC24; VWF | |
| 15% | 100.0% | 100.0% | 96.8% | 44 | APP; C14orf177; HHIP; HHIP-AS1; HOTAIRM1; HOXA- | 16 |
| | | | | | AS3; HOXA1; HOXA5; HOXA6; HOXC4; KIAA1161; LOC400043; ROBO2; | |
| | | | | | SLITRK5; TTC24; VWF | |
| 20% | 82.2% | 100.0% | 87.5% | 8 | HHIP; HHIP-AS1; HOTAIRM1; HOXA-AS3; HOXA5; SLITRK5 | 6 |
| 22% | 51.1% | 80.0% | 67.0% | 3 | HOXA-AS3; HOXA5 | 2 |

TABLE 4
Cross-validation results for different effect-size (absolute delta beta, |Δβ|) thresholds at p-value ≦0.001.
Shown are the specificity (Spec) and sensitivity (Sens) of the LOO procedure, specificity for 1056 normal blood samples
derived from GEO (Spec GEO). The total number of significant sites (CGs) in the resulting “CHD7 signature” set, the
gene names (Names) and their total number (Genes) corresponding to the significant sites. The p-values are corrected
for multiple testing (Benjamini-Hochberg correction).

| | p-value ≦0.001 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 99.7% | 210 | ALX3; ANO3; APP; ARHGEF15; ARPP21; ARSJ; BMP7; C14orf177; C1orf53; | 81 |
| | | | | | CAMTA1; COL11A1; COL4A2; COLEC12; DAB1; DLC1; EBF3; | |
| | | | | | ELAVL2; EPB41L1; FAM155A; FLJ12825; FMN2; FOXP1; FOXP2; | |
| | | | | | GPRC5C; HECW1; HHIP; HHIP-AS1; HOTAIRM1; HOXA- | |
| | | | | | AS3; HOXA1; HOXA10; HOXA10-HOXA9; HOXA11; HOXA11- | |
| | | | | | AS; HOXA2; HOXA5; HOXA6; HOXB8; IGF2; IGF2-AS; IL20RA; INS- | |
| | | | | | IGF2; ISL1; KCNQ4; KIRREL3; KLHL14; LINC00554; LINC00982; LMO3; | |
| | | | | | LOC400043; MIR10B; MIR1284; MKS1; MS4A6A; NFIB; NOX4; | |
| | | | | | OPRM1; PARVA; PCDH20; PGLYRP1; PKNOX2; PLBD1; PVRL3; | |
| | | | | | PVRL3- | |
| | | | | | AS1; RELN; RGS7; ROBO2; RUNX1T1; SGPP2; SLC1A3; SLCO1A2; | |
| | | | | | SLITRK5; TBX5; TEAD1; TENM4; TFAP2A; TMCC1; TMCC1- | |
| | | | | | AS1; TRUB1; VWF; WFDC2 | |
| 10% | 100.0% | 100.0% | 99.4% | 102 | HIP; HHIP-AS1; HOTAIRM1; HOXA- | 28 |
| | | | | | AS3; HOXA1; HOXA5; HOXA6; HOXB8; KIRREL3; LOC400043; MKS1; | |
| | | | | | NOX4; PARVA; PCDH20; ROBO2; SLCO1A2; SLITRK5; TBX5; TEAD1; | |
| | | | | | VWF | |
| 15% | 100.0% | 100.0% | 95.3% | 36 | APP; C14orf177; HHIP; HHIP-AS1; HOTAIRM1; HOXA- | 12 |
| | | | | | AS3; HOXA1; HOXA5; HOXA6; ROBO2; SLITRK5; VWF | |
| 20% | 82.2% | 100.0% | 87.5% | 8 | HHIP; HHIP-AS1; HOTAIRM1; HOXA-AS3; HOXA5; SLITRK5 | 6 |
| 22% | 51.1% | 80.0% | 67.0% | 3 | HOXA-AS3; HOXA5 | 2 |

TABLE 5
Cross-validation results for different effect-size (absolute delta beta, |Δβ|) thresholds at p-value ≦1e−4.
Shown are the specificity (Spec) and sensitivity (Sens) of the LOO procedure, specificity for 1056 normal blood samples
derived from GEO (Spec GEO). The total number of significant sites (CGs) in the resulting “CHD7 signature” set, the
gene names (Names) and their total number (Genes) corresponding to the significant sites. The p-values are corrected
for multiple testing (Benjamini-Hochberg correction).

| | p-value ≦1e−4 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 98.8% | 103 | APP; ARSJ; C14orf177; FAM155A; FOXP2; HOTAIRM1; HOXA- | 29 |
| | | | | | AS3; HOXA1; HOXA10; HOXA10-HOXA9; HOXA11; HOXA11- | |
| | | | | | AS; HOXA5; HOXA6; HOXB8; IL20RA; KIRREL3; MS4A6A; NOX4; | |
| | | | | | OPRM1; PARVA; PVRL3; PVRL3- | |
| | | | | | AS1; RELN; ROBO2; SLCO1A2; TBX5; TEAD1; VWF | |
| 10% | 100.0% | 100.0% | 98.5% | 72 | APP; C14orf177; FOXP2; HOTAIRM1; HOXA- | 17 |
| | | | | | AS3; HOXA1; HOXA5; HOXA6; HOXB8; KIRREL3; NOX4; PARVA; | |
| | | | | | ROBO2; SLCO1A2; TBX5; TEAD1; VWF | |
| 15% | 97.8% | 100.0% | 90.9% | 27 | APP; C14orf177; HOTAIRM1; HOXA- | 9 |
| | | | | | AS3; HOXA1; HOXA5; HOXA6; ROBO2; VWF | |
| 20% | 75.6% | 100.0% | 80.0% | 6 | HOTAIRM1; HOXA-AS3; HOXA5 | 3 |
| 22% | 48.9% | 66.7% | 67.0% | 3 | HOXA-AS3; HOXA5 | 2 |

TABLE 6
Cross-validation results for different effect-size (absolute delta beta, |Δβ|) thresholds at p-value ≦1e−5.
Shown are the specificity (Spec) and sensitivity (Sens) of the LOO procedure, specificity for 1056 normal blood samples
derived from GEO (Spec GEO). The total number of significant sites (CGs) in the resulting “CHD7 signature” set, the
gene names (Names) and their total number (Genes) corresponding to the significant sites. The p-values are corrected
for multiple testing (Benjamini-Hochberg correction).

| | p-value ≦1e−5 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 97.7% | 68 | APP; C14orf177; FOXP2; HOTAIRM1; HOXA- | 16 |
| | | | | | AS3; HOXA1; HOXA10; HOXA10- | |
| | | | | | HOXA9; HOXA5; HOXA6; HOXB8; PARVA; RELN; ROBO2; TBX5; | |
| | | | | | TEAD1 | |
| 10% | 100.0% | 100.0% | 97.7% | 53 | APP; C14orf177; FOXP2; HOTAIRM1; HOXA- | 13 |
| | | | | | AS3; HOXA1; HOXA5; HOXA6; HOXB8; PARVA; ROBO2; TBX5; TEAD1 | |
| 15% | 93.3% | 100.0% | 89.4% | 25 | APP; C14orf177; HOTAIRM1; HOXA- | 8 |
| | | | | | AS3; HOXA1; HOXA5; HOXA6; ROBO2 | |
| 20% | 75.6% | 100.0% | 80.0% | 6 | HOTAIRM1; HOXA-AS3; HOXA5 | 3 |
| 22% | 48.9% | 66.7% | 67.0% | 3 | HOXA-AS3; HOXA5 | 2 |

TABLE 7
KMT2D mutation information. Kabuki Score is defined by the formula:
KS score(B) = r (B, KS profile) − r (B, control profile)

| Sample ID | Sex | Nucleotide change | Amino acid change | Exon | Inheritance | Kabuki Score |
|---|---|---|---|---|---|---|
| P1 | F | c.15067C > T | p.R5021X | 48 | de novo | 0.357 |
| P2 | F | c.8171_8172del or 8172_8173del | p.P2724Qfs*5 | 32 | not in mom | 0.324 |
| P3 | M | c.6595del | p.Y2199Ifs*65 | 31 | de novo | 0.414 |
| P4 | M | c.14055-14056delCA | p.H4685Qfs*4 | 43 | de novo | 0.472 |
| P5 | M | c.6295C > T | p.R2099X | 31 | de novo | 0.250 |
| P6 | M | c.4135_4136del | p.M1379Vfs*52 | 14 | de novo | 0.415 |
| P7 | M | c.12592C > T | p.R4198X | 39 | de novo | 0.455 |
| P8 | M | c.4135_4136del | p.M1379VfsX*52 | 14 | de novo | 0.462 |
| P9 | M | c.11710C > T | p.Q3904X | 39 | de novo | 0.336 |
| P10 | M | c.16318delG | p.E5440Rfs*16 | 39 | de novo | 0.292 |
| P11 | M | c15030dupA | p.E5011Rfs*13 | 48 | de novo | 0.398 |
| U1 | F | | molecular pending | | | −0.212 |
| V1 | F | c.15143G > A | p.R5048H | 48 | unknown | 0.325 |
| V2 | M | c.12028 T > C | p.Ser4010Pro | 39 | unknown | −0.346 |
| V4 | M | c.15659G > A | p.R5220H | 48 | inherited | −0.308 |
| V5 | M | c.10256A > G | p.D3419G | 35 | inherited | −0.266 |
| V6 | F | c.8942G > A | p.E2992K | 34 | inherited | −0.349 |
| V7 | F | c.8831A > G | p.N2944S | 34 | inherited | −0.384 |
| V8 | F | c.832G > A | p.A278T | 6 | inherited | −0.281 |
| V9 | M | c.682C > G (known SNP) | p.R228G | 6 | inherited | −0.386 |

TABLE 8
Cross-validation results for different combination of statistical and effect-size thresholds. Shown are the specificity (Spec) and
sensitivity (Sens) of the LOO procedure, and the total number of significant sites (CGs) in the resulting “Kabuki signature”
set. One optimal combination was selected to be p-value ≦0.05 and |Δβ| >15%, which led to no classification
errors. Classification errors: FN = false negatives, FP = false positives.

| | | |
|---|---|---|
| p-value ≦0.05 | p-value ≦0.01 | p-value ≦0.005 |

| Db | Spec | FP | Sens | FN | CGs | Spec | FP | Sens | FN | CGs | Spec | FP | Sens | FN | CGs |
|---|---|---|---|---|---|---|---|---|---|---|---|---|---|---|---|
| 5% | 1 | | 0.91 | KP10 | 13595 | 1 | | 0.91 | KP10 | 9993 | 1 | | 0.91 | KP10 | 8490 |
| 10% | 1 | | 0.91 | KP10 | 1941 | 1 | | 0.91 | KP10 | 1704 | 1 | | 0.91 | KP10 | 1569 |
| 15% | 1 | | 1 | | 287 | 1 | | 1 | | 272 | 1 | | 1 | | 267 |
| 20% | 1 | | 1 | | 46 | 1 | | 1 | | 46 | 1 | | 1 | | 46 |
| 25% | 1 | | 0.55 | KP3 KP5 KP7 KP10 KP11 | 10 | 1 | | 0.82 | KP5 KP10 | 10 | 1 | | 0.91 | KP5 | 10 |

| | p-value ≦0.001 | | | | | p-value ≦ 0.0001 | | | | | p-value ≦0.00001 | | | | |
|---|---|---|---|---|---|---|---|---|---|---|---|---|---|---|---|
| Db | Spec | FP | Sens | FN | CGs | Spec | FP | Sens | FN | CGs | Spec | FP | Sens | FN | CGs |
| 5% | 1 | | 0.91 | KP10 | 5492 | 1 | | 1 | | 2696 | 1 | | 1 | | 1188 |
| 10% | 1 | | 1 | | 1248 | 1 | | 1 | | 801 | 1 | | 1 | | 447 |
| 15% | 1 | | 1 | | 232 | 1 | | 1 | | 181 | 1 | | 1 | | 111 |
| 20% | 1 | | 1 | | 43 | 1 | | 1 | | 39 | 1 | | 1 | | 29 |
| 25% | 1 | | 0.91 | KP5 | 9 | 1 | | 0.82 | KP2 KP5 | 9 | 1 | | 0.91 | KP10 | 6 |

TABLE 9
287 CpG loci corresponding to 162 genes were identified as showing a statistically significant (p-value ≦0.05) difference in KS
and non-KS controls. “Mean not-Kabuki” refers to the mean beta-value for the CpG loci in the non-KS cases. “Mean Kabuki” refers to the
mean beta-value for the CpG loci in the KS samples.

| | | Benjamini- | | | | | | | |
|---|---|---|---|---|---|---|---|---|---|
| | | Hochberg | | | DNA | | | | |
| | | corrected p- | | Absolute | methylation | Mean not- | Mean | | |
| Illumina ID | p-value | value | deltaBeta | deltaBeta | effect | Kabuki | Kabuki | Gene Symbol | Genome_Build |
| cg22987448 | 5.37E−11 | 1.44E−07 | −0.368 | 0.368 | LOSS | 0.857 | 0.490 | MYO1F | 37 |
| cg15254671 | 2.69E−11 | 1.11E−07 | −0.344 | 0.344 | LOSS | 0.828 | 0.484 | MYO1F | 37 |
| cg05857996 | 2.03E−07 | 1.56E−05 | −0.335 | 0.335 | LOSS | 0.693 | 0.358 | EBF4 | 37 |
| cg08283130 | 2.55E−10 | 2.92E−07 | −0.280 | 0.280 | LOSS | 0.827 | 0.547 | MYO1F | 37 |
| cg01178624 | 2.03E−07 | 1.56E−05 | −0.278 | 0.278 | LOSS | 0.795 | 0.516 | KCNK7; KCNK7; KCNK7; | 37 |
| | | | | | | | | KCNK7 | |
| cg00274965 | 4.44E−07 | 2.71E−05 | −0.272 | 0.272 | LOSS | 0.361 | 0.089 | | 37 |
| cg09232555 | 0.000373661 | 0.003497351 | −0.264 | 0.264 | LOSS | 0.593 | 0.329 | C8orf49 | 37 |
| cg22568423 | 9.40E−11 | 1.80E−07 | −0.259 | 0.259 | LOSS | 0.793 | 0.534 | MYO1F | 37 |
| cg08818610 | 9.00E−09 | 2.04E−06 | 0.259 | 0.259 | GAIN | 0.347 | 0.606 | FAM65B; FAM65B; | 37 |
| | | | | | | | | FAM65B | |
| cg16370398 | 5.01E−10 | 4.23E−07 | −0.250 | 0.250 | LOSS | 0.499 | 0.248 | HOXC4; HOXC4 | 37 |
| cg15954353 | 5.56E−05 | 0.000865565 | −0.248 | 0.248 | LOSS | 0.776 | 0.529 | LOC728392 | 37 |
| cg05825244 | 5.49E−08 | 6.50E−06 | −0.246 | 0.246 | LOSS | 0.332 | 0.086 | EBF4 | 37 |
| cg09226051 | 2.24E−06 | 8.63E−05 | −0.243 | 0.243 | LOSS | 0.427 | 0.185 | NLRP3; NLRP3; NLRP3; | 37 |
| | | | | | | | | NLRP3; NLRP3; | |
| | | | | | | | | NLRP3 | |
| cg21637392 | 2.04E−08 | 3.44E−06 | 0.239 | 0.239 | GAIN | 0.098 | 0.337 | RNF216; RNF216 | 37 |
| cg14172108 | 4.44E−07 | 2.71E−05 | −0.236 | 0.236 | LOSS | 0.508 | 0.272 | | 37 |
| cg11532431 | 6.04E−10 | 4.32E−07 | −0.233 | 0.233 | LOSS | 0.833 | 0.600 | HOXA4 | 37 |
| cg20543544 | 3.26E−09 | 1.18E−06 | 0.229 | 0.229 | GAIN | 0.294 | 0.523 | ZMIZ1 | 37 |
| cg05491854 | 2.04E−08 | 3.44E−06 | 0.226 | 0.226 | GAIN | 0.485 | 0.711 | FAM65B; FAM65B; | 37 |
| | | | | | | | | FAM65B | |
| cg08255475 | 2.55E−10 | 2.92E−07 | −0.226 | 0.226 | LOSS | 0.518 | 0.292 | CDT1 | 37 |
| cg08425810 | 3.02E−07 | 2.07E−05 | −0.226 | 0.226 | LOSS | 0.729 | 0.503 | AGAP2; AGAP2 | 37 |
| cg22997113 | 9.00E−09 | 2.04E−06 | −0.225 | 0.225 | LOSS | 0.592 | 0.367 | HOXA4; HOXA4 | 37 |
| cg15454820 | 5.49E−08 | 6.50E−06 | 0.224 | 0.224 | GAIN | 0.213 | 0.437 | | 37 |
| cg14911689 | 0.000496589 | 0.004303102 | 0.224 | 0.224 | GAIN | 0.389 | 0.613 | NINJ2 | 37 |
| cg25308803 | 1.57E−08 | 2.89E−06 | −0.224 | 0.224 | LOSS | 0.622 | 0.398 | SH3RF3; SH3RF3- | 37 |
| | | | | | | | | AS1; SH3RF3-AS1 | |
| cg10785373 | 6.73E−09 | 1.68E−06 | −0.223 | 0.223 | LOSS | 0.587 | 0.364 | | 37 |
| cg23387569 | 4.03E−10 | 3.55E−07 | −0.221 | 0.221 | LOSS | 0.867 | 0.645 | AGAP2; AGAP2; AGAP2- | 37 |
| | | | | | | | | AS1 | |
| cg00313914 | 9.15E−07 | 4.51E−05 | −0.220 | 0.220 | LOSS | 0.532 | 0.312 | NAV1 | 37 |
| cg03846641 | 3.26E−09 | 1.18E−06 | −0.218 | 0.218 | LOSS | 0.602 | 0.384 | SH3RF3; SH3RF3- | 37 |
| | | | | | | | | AS1; SH3RF3-AS1 | |
| cg14099457 | 1.19E−08 | 2.44E−06 | 0.217 | 0.217 | GAIN | 0.534 | 0.752 | LAMB2; LAMB2 | 37 |
| cg19738980 | 1.30E−09 | 6.59E−07 | −0.216 | 0.216 | LOSS | 0.621 | 0.405 | LAMA1 | 37 |
| cg19142026 | 3.02E−07 | 2.07E−05 | −0.215 | 0.215 | LOSS | 0.320 | 0.105 | HOXA4; HOXA4 | 37 |
| cg09549073 | 1.08E−07 | 1.02E−05 | 0.215 | 0.215 | GAIN | 0.589 | 0.803 | HOXA5; HOXA5; HOXA- | 37 |
| | | | | | | | | AS3 | |
| cg04287574 | 3.45E−05 | 0.000609982 | −0.213 | 0.213 | LOSS | 0.379 | 0.165 | NAV1 | 37 |
| cg03269218 | 9.00E−09 | 2.04E−06 | 0.211 | 0.211 | GAIN | 0.320 | 0.530 | | 37 |
| cg05905531 | 4.03E−10 | 3.55E−07 | −0.207 | 0.207 | LOSS | 0.820 | 0.612 | MYO1F | 37 |
| cg12474798 | 3.64E−09 | 1.18E−06 | −0.207 | 0.207 | LOSS | 0.479 | 0.272 | ADO | 37 |
| cg20225999 | 9.00E−09 | 2.04E−06 | −0.206 | 0.206 | LOSS | 0.819 | 0.613 | | 37 |
| cg24690094 | 7.00E−05 | 0.001021959 | 0.206 | 0.206 | GAIN | 0.462 | 0.668 | DOC2GP | 37 |
| cg02224314 | 9.00E−10 | 5.32E−07 | 0.205 | 0.205 | GAIN | 0.710 | 0.916 | BCL11B; BCL11B; | 37 |
| | | | | | | | | BCL11B; BCL11B | |
| cg18025886 | 4.03E−10 | 3.55E−07 | 0.204 | 0.204 | GAIN | 0.524 | 0.728 | MFI2; MFI2 | 37 |
| cg03146625 | 3.64E−09 | 1.18E−06 | −0.204 | 0.204 | LOSS | 0.573 | 0.369 | HOXC4; HOXC4 | 37 |
| cg21429551 | 3.39E−08 | 4.75E−06 | −0.204 | 0.204 | LOSS | 0.504 | 0.301 | GARS | 37 |
| cg03455316 | 3.45E−05 | 0.000609982 | 0.203 | 0.203 | GAIN | 0.616 | 0.819 | | 37 |
| cg06663305 | 1.65E−07 | 1.36E−05 | 0.203 | 0.203 | GAIN | 0.282 | 0.485 | | 37 |
| cg09817024 | 3.39E−08 | 4.75E−06 | 0.202 | 0.202 | GAIN | 0.178 | 0.379 | | 37 |
| cg09214243 | 6.13E−06 | 0.000175468 | 0.201 | 0.201 | GAIN | 0.516 | 0.717 | | 37 |
| cg01246520 | 7.84E−05 | 0.001110451 | 0.200 | 0.200 | GAIN | 0.529 | 0.729 | RAI1 | 37 |
| cg26404511 | 2.69E−11 | 1.11E−07 | −0.199 | 0.199 | LOSS | 0.320 | 0.121 | CNR2 | 37 |
| cg15795305 | 9.00E−10 | 5.32E−07 | 0.198 | 0.198 | GAIN | 0.314 | 0.512 | | 37 |
| cg14018024 | 3.45E−05 | 0.000609982 | −0.198 | 0.198 | LOSS | 0.721 | 0.523 | LAMC3 | 37 |
| cg20704450 | 3.67E−07 | 2.37E−05 | 0.198 | 0.198 | GAIN | 0.399 | 0.596 | | 37 |
| cg14759565 | 2.64E−08 | 4.05E−06 | −0.197 | 0.197 | LOSS | 0.835 | 0.637 | | 37 |
| cg24263062 | 5.34E−07 | 3.08E−05 | −0.197 | 0.197 | LOSS | 0.565 | 0.368 | EBF4 | 37 |
| cg26654770 | 0.004922514 | 0.022926275 | 0.197 | 0.197 | GAIN | 0.373 | 0.569 | NINJ2 | 37 |
| cg12128839 | 5.49E−08 | 6.50E−06 | 0.197 | 0.197 | GAIN | 0.621 | 0.818 | HOXA5; HOXA5; HOXA- | 37 |
| | | | | | | | | AS3 | |
| cg04517524 | 6.73E−09 | 1.68E−06 | −0.196 | 0.196 | LOSS | 0.476 | 0.279 | ASB2; ASB2 | 37 |
| cg11015251 | 5.34E−07 | 3.08E−05 | −0.196 | 0.196 | LOSS | 0.461 | 0.265 | HOXA4; HOXA4 | 37 |
| cg11693285 | 3.89E−05 | 0.000666588 | 0.196 | 0.196 | GAIN | 0.301 | 0.497 | | 37 |
| cg24217894 | 1.34E−11 | 9.45E−08 | −0.196 | 0.196 | LOSS | 0.876 | 0.680 | AGAP2; AGAP2; AGAP2- | 37 |
| | | | | | | | | AS1 | |
| cg24680632 | 5.50E−08 | 6.51E−06 | 0.196 | 0.196 | GAIN | 0.239 | 0.435 | | 37 |
| cg08347626 | 3.67E−07 | 2.37E−05 | 0.195 | 0.195 | GAIN | 0.433 | 0.628 | | 37 |
| cg23901918 | 3.39E−08 | 4.75E−06 | −0.195 | 0.195 | LOSS | 0.353 | 0.158 | SH3PXD2A | 37 |
| cg06847624 | 4.33E−08 | 5.55E−06 | −0.195 | 0.195 | LOSS | 0.315 | 0.121 | PFN3; PFN3 | 37 |
| cg03068497 | 5.49E−08 | 6.50E−06 | −0.194 | 0.194 | LOSS | 0.553 | 0.359 | GARS | 37 |
| cg00815832 | 1.57E−08 | 2.89E−06 | 0.194 | 0.194 | GAIN | 0.567 | 0.761 | | 37 |
| cg27403406 | 1.23E−05 | 0.000291253 | −0.194 | 0.194 | LOSS | 0.659 | 0.465 | B4GALT5 | 37 |
| cg01994308 | 9.15E−07 | 4.51E−05 | 0.194 | 0.194 | GAIN | 0.401 | 0.594 | CHCHD7; CHCHD7; | 37 |
| | | | | | | | | CHCHD7; CHCHD7; | |
| | | | | | | | | CHCHD7; PLAG1; | |
| | | | | | | | | PLAG1; PLAG1; | |
| | | | | | | | | CHCHD7 | |
| cg05991492 | 2.69E−05 | 0.000512459 | 0.193 | 0.193 | GAIN | 0.410 | 0.604 | | 37 |
| cg13379325 | 0.002931544 | 0.015698881 | −0.193 | 0.193 | LOSS | 0.694 | 0.501 | KCNQ2; KCNQ2; KCNQ2; | 37 |
| | | | | | | | | KCNQ2 | |
| cg23549902 | 5.56E−05 | 0.000865565 | 0.193 | 0.193 | GAIN | 0.487 | 0.680 | ZNF890P; ZNF890P | 37 |
| cg00401101 | 2.11E−06 | 8.17E−05 | −0.193 | 0.193 | LOSS | 0.432 | 0.239 | FAM134B; FAM134B | 37 |
| cg14845962 | 1.61E−10 | 2.37E−07 | −0.193 | 0.193 | LOSS | 0.936 | 0.744 | AGAP2; AGAP2; AGAP2- | 37 |
| | | | | | | | | AS1 | |
| cg23669081 | 0.003163359 | 0.016600183 | −0.192 | 0.192 | LOSS | 0.544 | 0.351 | HOXB7 | 37 |
| cg16651126 | 3.39E−08 | 4.75E−06 | −0.192 | 0.192 | LOSS | 0.392 | 0.200 | HOXA4; HOXA4 | 37 |
| cg11336382 | 7.67E−07 | 3.99E−05 | 0.192 | 0.192 | GAIN | 0.481 | 0.673 | | 37 |
| cg00130223 | 3.08E−09 | 1.13E−06 | −0.191 | 0.191 | LOSS | 0.555 | 0.364 | | 37 |
| cg06904356 | 3.02E−07 | 2.07E−05 | 0.191 | 0.191 | GAIN | 0.674 | 0.864 | | 37 |
| cg01238044 | 0.001641905 | 0.010412048 | 0.191 | 0.191 | GAIN | 0.173 | 0.364 | GSTT1; GSTT1 | 37 |
| cg07211044 | 5.37E−11 | 1.44E−07 | −0.190 | 0.190 | LOSS | 0.440 | 0.250 | TOX | 37 |
| cg24927841 | 1.87E−09 | 8.15E−07 | −0.190 | 0.190 | LOSS | 0.761 | 0.570 | | 37 |
| cg10146935 | 2.64E−08 | 4.05E−06 | −0.190 | 0.190 | LOSS | 0.275 | 0.084 | SAMD11 | 37 |
| cg19579217 | 9.00E−09 | 2.04E−06 | 0.190 | 0.190 | GAIN | 0.560 | 0.750 | | 37 |
| cg25910261 | 0.000109302 | 0.001420029 | 0.190 | 0.190 | GAIN | 0.281 | 0.471 | PTPRN2; PTPRN2; | 37 |
| | | | | | | | | PTPRN2 | |
| cg17740434 | 2.55E−10 | 2.92E−07 | 0.190 | 0.190 | GAIN | 0.308 | 0.498 | MIR548N; TTN- | 37 |
| | | | | | | | | AS1; TTN-AS1 | |
| cg02919082 | 0.001110315 | 0.007731233 | −0.189 | 0.189 | LOSS | 0.481 | 0.291 | HLA-DQA1 | 37 |
| cg02616966 | 4.29E−05 | 0.000728646 | 0.189 | 0.189 | GAIN | 0.059 | 0.249 | MCCC1; MCCC1 | 37 |
| cg11510586 | 1.61E−05 | 0.000353081 | 0.188 | 0.188 | GAIN | 0.258 | 0.447 | | 37 |
| cg20100745 | 0.000206017 | 0.002263976 | −0.188 | 0.188 | LOSS | 0.459 | 0.271 | NDRG1; NDRG1; NDRG1; | 37 |
| | | | | | | | | NDRG1 | |
| cg02715602 | 4.33E−08 | 5.55E−06 | −0.188 | 0.188 | LOSS | 0.850 | 0.663 | SEMA6B | 37 |
| cg07599786 | 3.38E−06 | 0.000114817 | −0.187 | 0.187 | LOSS | 0.544 | 0.357 | NAV1 | 37 |
| cg16423910 | 9.40E−11 | 1.80E−07 | 0.186 | 0.186 | GAIN | 0.345 | 0.531 | CD37; CD37 | 37 |
| cg08911368 | 6.91E−08 | 7.57E−06 | 0.186 | 0.186 | GAIN | 0.142 | 0.328 | | 37 |
| cg03930209 | 1.30E−09 | 6.59E−07 | 0.185 | 0.185 | GAIN | 0.617 | 0.802 | | 37 |
| cg16440561 | 2.48E−06 | 9.16E−05 | −0.185 | 0.185 | LOSS | 0.277 | 0.092 | SPEG | 37 |
| cg23489137 | 0.001008525 | 0.007271153 | −0.185 | 0.185 | LOSS | 0.530 | 0.345 | RBMS1; RBMS1 | 37 |
| cg02639108 | 3.38E−06 | 0.000114817 | −0.185 | 0.185 | LOSS | 0.731 | 0.547 | | 37 |
| cg11410718 | 5.34E−07 | 3.08E−05 | −0.184 | 0.184 | LOSS | 0.412 | 0.228 | HOXA4; HOXA4 | 37 |
| cg05463589 | 1.34E−07 | 1.17E−05 | 0.183 | 0.183 | GAIN | 0.623 | 0.806 | IL17C | 37 |
| cg16823042 | 2.51E−10 | 2.92E−07 | −0.183 | 0.183 | LOSS | 0.718 | 0.535 | AGAP2; AGAP2; AGAP2- | 37 |
| | | | | | | | | AS1 | |
| cg03613822 | 8.16E−06 | 0.000215408 | −0.183 | 0.183 | LOSS | 0.662 | 0.479 | DLG4; DLG4 | 37 |
| cg24652615 | 1.80E−06 | 7.28E−05 | −0.183 | 0.183 | LOSS | 0.682 | 0.500 | TMEM151B | 37 |
| cg16565409 | 1.30E−09 | 6.59E−07 | −0.182 | 0.182 | LOSS | 0.512 | 0.330 | RPL23A; SNORD4 | 37 |
| cg13518079 | 2.62E−09 | 9.86E−07 | −0.182 | 0.182 | LOSS | 0.276 | 0.094 | EBF4 | 37 |
| cg02892925 | 2.69E−11 | 1.11E−07 | −0.182 | 0.182 | LOSS | 0.636 | 0.454 | TOX | 37 |
| cg17569124 | 1.65E−07 | 1.36E−05 | 0.181 | 0.181 | GAIN | 0.625 | 0.806 | HOXA5; HOXA- | 37 |
| | | | | | | | | AS3 | |
| cg19196401 | 8.16E−06 | 0.000215408 | 0.181 | 0.181 | GAIN | 0.645 | 0.826 | DDO; DDO | 37 |
| cg13068698 | 1.02E−05 | 0.000257113 | −0.181 | 0.181 | LOSS | 0.414 | 0.233 | DPY19L1 | 37 |
| cg07317062 | 1.29E−06 | 5.75E−05 | −0.181 | 0.181 | LOSS | 0.397 | 0.217 | HOXA4; HOXA4 | 37 |
| cg10648815 | 5.34E−07 | 3.08E−05 | −0.180 | 0.180 | LOSS | 0.647 | 0.467 | LAIR2; LAIR2 | 37 |
| cg03651054 | 0.002512273 | 0.014035499 | −0.180 | 0.180 | LOSS | 0.620 | 0.440 | | 37 |
| cg16814680 | 0.006594781 | 0.028440415 | −0.180 | 0.180 | LOSS | 0.525 | 0.345 | | 37 |
| cg12097883 | 1.61E−10 | 2.37E−07 | −0.180 | 0.180 | LOSS | 0.293 | 0.113 | LOC146880; LOC146880 | 37 |
| cg23061725 | 1.19E−08 | 2.44E−06 | 0.179 | 0.179 | GAIN | 0.344 | 0.523 | CASP8; CASP8; CASP8; | 37 |
| | | | | | | | | CASP8; CASP8; | |
| | | | | | | | | CASP8 | |
| cg14359292 | 4.44E−07 | 2.71E−05 | −0.179 | 0.179 | LOSS | 0.322 | 0.143 | HOXA4 | 37 |
| cg18424841 | 5.34E−07 | 3.08E−05 | −0.179 | 0.179 | LOSS | 0.739 | 0.560 | | 37 |
| cg04991337 | 3.45E−05 | 0.000609982 | 0.178 | 0.178 | GAIN | 0.106 | 0.284 | MCCC1; MCCC1 | 37 |
| cg02439789 | 1.52E−06 | 6.46E−05 | −0.178 | 0.178 | LOSS | 0.472 | 0.294 | SAMD11 | 37 |
| cg01948217 | 1.08E−07 | 1.02E−05 | 0.178 | 0.178 | GAIN | 0.243 | 0.421 | BPI | 37 |
| cg12748890 | 2.48E−06 | 9.16E−05 | −0.178 | 0.178 | LOSS | 0.732 | 0.554 | SYTL1; SYTL1 | 37 |
| cg11969813 | 1.08E−07 | 1.02E−05 | −0.178 | 0.178 | LOSS | 0.821 | 0.644 | P4HB | 37 |
| cg18977541 | 4.33E−08 | 5.55E−06 | 0.177 | 0.177 | GAIN | 0.169 | 0.347 | | 37 |
| cg16734913 | 5.56E−05 | 0.000865565 | −0.177 | 0.177 | LOSS | 0.665 | 0.487 | OR5W2 | 37 |
| cg22220710 | 2.04E−08 | 3.44E−06 | −0.177 | 0.177 | LOSS | 0.605 | 0.428 | LAMA1 | 37 |
| cg03604073 | 6.41E−07 | 3.50E−05 | 0.177 | 0.177 | GAIN | 0.243 | 0.420 | ARHGAP35 | 37 |
| cg25513090 | 8.66E−08 | 8.79E−06 | −0.176 | 0.176 | LOSS | 0.702 | 0.526 | DAGLB; DAGLB | 37 |
| cg26823666 | 1.65E−07 | 1.36E−05 | 0.176 | 0.176 | GAIN | 0.316 | 0.492 | | 37 |
| cg20016023 | 9.00E−10 | 5.32E−07 | −0.176 | 0.176 | LOSS | 0.501 | 0.325 | RRP12; RRP12; RRP12 | 37 |
| cg24753998 | 6.13E−06 | 0.000175468 | 0.176 | 0.176 | GAIN | 0.416 | 0.593 | MAP3K7CL; MAP3K7CL; | 37 |
| | | | | | | | | MAP3K7CL; | |
| | | | | | | | | MAP3K7CL; MAP3K7CL; | |
| | | | | | | | | MAP3K7CL | |
| cg26371957 | 0.005673932 | 0.025448023 | 0.176 | 0.176 | GAIN | 0.467 | 0.643 | NINJ2 | 37 |
| cg25307665 | 1.80E−06 | 7.28E−05 | 0.174 | 0.174 | GAIN | 0.654 | 0.828 | HOXA5; HOXA- | 37 |
| | | | | | | | | AS3 | |
| cg20978937 | 6.91E−08 | 7.57E−06 | −0.174 | 0.174 | LOSS | 0.535 | 0.361 | PLD4 | 37 |
| cg08234664 | 1.29E−06 | 5.75E−05 | 0.174 | 0.174 | GAIN | 0.499 | 0.674 | LAMB2; LAMB2 | 37 |
| cg13619522 | 2.54E−08 | 4.05E−06 | −0.174 | 0.174 | LOSS | 0.747 | 0.573 | CSK; CSK | 37 |
| cg20698421 | 4.33E−08 | 5.55E−06 | −0.174 | 0.174 | LOSS | 0.555 | 0.381 | SLC1A4; SLC1A4 | 37 |
| cg11511175 | 7.52E−10 | 5.32E−07 | −0.173 | 0.173 | LOSS | 0.752 | 0.578 | AGAP2; AGAP2; AGAP2- | 37 |
| | | | | | | | | AS1 | |
| cg27001715 | 6.83E−08 | 7.57E−06 | −0.173 | 0.173 | LOSS | 0.552 | 0.379 | | 37 |
| cg19916659 | 1.80E−06 | 7.28E−05 | 0.173 | 0.173 | GAIN | 0.295 | 0.467 | MIR548N; TTN- | 37 |
| | | | | | | | | AS1; TTN-AS1 | |
| cg07616871 | 2.62E−09 | 9.86E−07 | −0.173 | 0.173 | LOSS | 0.639 | 0.467 | | 37 |
| cg21476494 | 3.64E−09 | 1.18E−06 | 0.172 | 0.172 | GAIN | 0.360 | 0.532 | | 37 |
| cg20007021 | 1.29E−06 | 5.75E−05 | 0.172 | 0.172 | GAIN | 0.233 | 0.405 | LTB4R; LTB4R2; CIDEB; | 37 |
| | | | | | | | | CIDEB; LTB4R2 | |
| cg10044179 | 0.002714796 | 0.014844937 | −0.171 | 0.171 | LOSS | 0.461 | 0.289 | ANKRD20A11P | 37 |
| cg12176783 | 7.67E−07 | 3.99E−05 | −0.171 | 0.171 | LOSS | 0.651 | 0.480 | TCEA2; TCEA2 | 37 |
| cg02954987 | 1.65E−07 | 1.36E−05 | 0.171 | 0.171 | GAIN | 0.583 | 0.754 | LAMB2; LAMB2 | 37 |
| cg21570209 | 5.30E−06 | 0.000157903 | 0.171 | 0.171 | GAIN | 0.490 | 0.661 | FOXA3; SYMPK | 37 |
| cg24937727 | 1.08E−07 | 1.02E−05 | −0.171 | 0.171 | LOSS | 0.265 | 0.094 | RGL3; RGL3 | 37 |
| cg22582187 | 6.73E−09 | 1.68E−06 | −0.171 | 0.171 | LOSS | 0.760 | 0.589 | | 37 |
| cg03043406 | 1.34E−11 | 9.45E−08 | −0.170 | 0.170 | LOSS | 0.614 | 0.444 | RPS8; SNORD38A | 37 |
| cg09636302 | 2.11E−06 | 8.17E−05 | −0.170 | 0.170 | LOSS | 0.734 | 0.564 | HAL; HAL; HAL | 37 |
| cg24550112 | 3.64E−09 | 1.18E−06 | −0.170 | 0.170 | LOSS | 0.337 | 0.167 | PRDM2 | 37 |
| cg17900689 | 5.49E−08 | 6.50E−06 | −0.170 | 0.170 | LOSS | 0.668 | 0.499 | ZMYND15; ZMYND15; | 37 |
| | | | | | | | | ZMYND15 | |
| cg24524285 | 1.19E−08 | 2.44E−06 | −0.170 | 0.170 | LOSS | 0.652 | 0.482 | NRXN2; NRXN2; NRXN2 | 37 |
| cg17655970 | 9.79E−05 | 0.001309695 | 0.169 | 0.169 | GAIN | 0.293 | 0.462 | | 37 |
| cg24194775 | 1.84E−05 | 0.000387482 | −0.169 | 0.169 | LOSS | 0.546 | 0.377 | NPR2 | 37 |
| cg04387835 | 0.000150821 | 0.001799142 | −0.169 | 0.169 | LOSS | 0.463 | 0.294 | ZMYND15; ZMYND15; | 37 |
| | | | | | | | | ZMYND15 | |
| cg26411441 | 6.91E−08 | 7.57E−06 | −0.168 | 0.168 | LOSS | 0.458 | 0.289 | HSPA12B; HSPA12B | 37 |
| cg24517467 | 1.52E−06 | 6.46E−05 | 0.168 | 0.168 | GAIN | 0.458 | 0.627 | | 37 |
| cg08657492 | 3.02E−07 | 2.07E−05 | −0.168 | 0.168 | LOSS | 0.510 | 0.341 | HOXA4 | 37 |
| cg17431280 | 9.79E−05 | 0.001309695 | 0.168 | 0.168 | GAIN | 0.184 | 0.352 | ARHGAP35 | 37 |
| cg20748533 | 2.90E−06 | 0.000102762 | −0.168 | 0.168 | LOSS | 0.455 | 0.287 | SHANK1 | 37 |
| cg21111256 | 0.007480707 | 0.031135633 | 0.168 | 0.168 | GAIN | 0.320 | 0.489 | CYP2A7; CYP2A7 | 37 |
| cg06768599 | 1.34E−11 | 9.45E−08 | −0.168 | 0.168 | LOSS | 0.951 | 0.783 | LTB4R; LTB4R | 37 |
| cg18090145 | 1.52E−06 | 6.46E−05 | −0.168 | 0.168 | LOSS | 0.712 | 0.544 | | 37 |
| cg00343839 | 7.08E−06 | 0.000194415 | −0.168 | 0.168 | LOSS | 0.337 | 0.169 | LOC728392 | 37 |
| cg00290607 | 9.79E−05 | 0.001309695 | 0.168 | 0.168 | GAIN | 0.640 | 0.807 | DOC2GP | 37 |
| cg00011856 | 8.66E−08 | 8.79E−06 | −0.168 | 0.168 | LOSS | 0.528 | 0.360 | IGFBP5 | 37 |
| cg24940967 | 1.19E−08 | 2.44E−06 | −0.167 | 0.167 | LOSS | 0.455 | 0.288 | SLC6A20; SLC6A20 | 37 |
| cg15265092 | 6.24E−05 | 0.000941319 | −0.167 | 0.167 | LOSS | 0.640 | 0.473 | SNRPC; SNRPC | 37 |
| cg25288140 | 6.82E−05 | 0.001021959 | −0.167 | 0.167 | LOSS | 0.799 | 0.633 | BRCA1; BRCA1; BRCA1; | 37 |
| | | | | | | | | BRCA1; NBR2; | |
| | | | | | | | | BRCA1 | |
| cg19786602 | 0.000150821 | 0.001799142 | −0.167 | 0.167 | LOSS | 0.571 | 0.404 | | 37 |
| cg13614409 | 7.84E−05 | 0.001110451 | −0.167 | 0.167 | LOSS | 0.611 | 0.445 | TEX26; TEX26- | 37 |
| | | | | | | | | AS1; TEX26- | |
| | | | | | | | | AS1; TEX26- | |
| | | | | | | | | AS1; TEX26-AS1 | |
| cg27539527 | 1.61E−10 | 2.37E−07 | 0.167 | 0.167 | GAIN | 0.484 | 0.651 | | 37 |
| cg01834979 | 1.61E−10 | 2.37E−07 | −0.166 | 0.166 | LOSS | 0.838 | 0.671 | AGAP2; AGAP2; AGAP2- | 37 |
| | | | | | | | | AS1 | |
| cg23060513 | 3.94E−06 | 0.000127744 | −0.166 | 0.166 | LOSS | 0.767 | 0.601 | FARSA | 37 |
| cg08739651 | 2.55E−10 | 2.92E−07 | 0.166 | 0.166 | GAIN | 0.229 | 0.395 | FLJ31813 | 37 |
| cg08355456 | 3.05E−05 | 0.000559351 | 0.166 | 0.166 | GAIN | 0.498 | 0.664 | DOC2GP | 37 |
| cg18322589 | 4.33E−08 | 5.55E−06 | 0.166 | 0.166 | GAIN | 0.713 | 0.879 | TACC2; TACC2 | 37 |
| cg24576298 | 3.02E−07 | 2.07E−05 | −0.166 | 0.166 | LOSS | 0.632 | 0.466 | PNPLA8; PNPLA8; | 37 |
| | | | | | | | | PNPLA8; PNPLA8; | |
| | | | | | | | | PNPLA8; PNPLA8 | |
| cg06015422 | 0.001919305 | 0.011652497 | 0.166 | 0.166 | GAIN | 0.293 | 0.459 | | 37 |
| cg22259797 | 0.001982236 | 0.011800854 | −0.165 | 0.165 | LOSS | 0.532 | 0.366 | C2CD2L | 37 |
| cg18587137 | 3.29E−08 | 4.75E−06 | −0.165 | 0.165 | LOSS | 0.882 | 0.716 | TNFAIP2 | 37 |
| cg18784409 | 4.44E−07 | 2.71E−05 | −0.165 | 0.165 | LOSS | 0.517 | 0.352 | CHKA; CHKA | 37 |
| cg13759905 | 3.64E−09 | 1.18E−06 | −0.164 | 0.164 | LOSS | 0.426 | 0.262 | | 37 |
| cg09284949 | 1.65E−07 | 1.36E−05 | −0.164 | 0.164 | LOSS | 0.250 | 0.086 | SHANK1 | 37 |
| cg03701930 | 5.30E−06 | 0.000157903 | −0.164 | 0.164 | LOSS | 0.259 | 0.095 | | 37 |
| cg03691722 | 1.65E−07 | 1.36E−05 | −0.164 | 0.164 | LOSS | 0.666 | 0.502 | LAMA1 | 37 |
| cg27466845 | 4.33E−08 | 5.55E−06 | −0.164 | 0.164 | LOSS | 0.819 | 0.656 | NRXN2; NRXN2; NRXN2 | 37 |
| cg16915863 | 4.44E−07 | 2.71E−05 | −0.163 | 0.163 | LOSS | 0.772 | 0.608 | LOC400043 | 37 |
| cg12133451 | 6.24E−05 | 0.000941319 | 0.163 | 0.163 | GAIN | 0.614 | 0.778 | | 37 |
| cg06137123 | 0.000109302 | 0.001420029 | −0.163 | 0.163 | LOSS | 0.741 | 0.578 | | 37 |
| cg10501093 | 1.19E−08 | 2.44E−06 | −0.163 | 0.163 | LOSS | 0.919 | 0.756 | TNFAIP2 | 37 |
| cg08801017 | 8.16E−06 | 0.000215408 | −0.163 | 0.163 | LOSS | 0.504 | 0.341 | ANKRD26P3; LINC00421 | 37 |
| cg16312514 | 0.00059748 | 0.004922032 | −0.162 | 0.162 | LOSS | 0.503 | 0.341 | SHANK2 | 37 |
| cg02666610 | 1.23E−05 | 0.000291253 | −0.162 | 0.162 | LOSS | 0.284 | 0.122 | | 37 |
| cg19321684 | 2.64E−08 | 4.05E−06 | 0.162 | 0.162 | GAIN | 0.319 | 0.481 | GPSM3; GPSM3 | 37 |
| cg05076221 | 4.33E−08 | 5.55E−06 | 0.161 | 0.161 | GAIN | 0.570 | 0.731 | HOXA5; HOXA- | 37 |
| | | | | | | | | AS3 | |
| cg23502204 | 0.000278724 | 0.002820594 | −0.161 | 0.161 | LOSS | 0.666 | 0.504 | RAB38 | 37 |
| cg20299697 | 3.94E−06 | 0.000127744 | −0.161 | 0.161 | LOSS | 0.618 | 0.457 | MRAS; MRAS; MRAS; | 37 |
| | | | | | | | | MRAS; MRAS; | |
| | | | | | | | | MRAS | |
| cg07040013 | 0.000109302 | 0.001420029 | 0.161 | 0.161 | GAIN | 0.589 | 0.751 | | 37 |
| cg07509935 | 2.48E−06 | 9.16E−05 | 0.161 | 0.161 | GAIN | 0.239 | 0.400 | LTB4R; LTB4R2; CIDEB; | 37 |
| | | | | | | | | LTB4R2 | |
| cg01231141 | 3.38E−06 | 0.000114817 | 0.161 | 0.161 | GAIN | 0.512 | 0.673 | ADAMTS2; ADAMTS2 | 37 |
| cg22127848 | 9.38E−06 | 0.000238239 | −0.161 | 0.161 | LOSS | 0.672 | 0.511 | | 37 |
| cg04015962 | 1.84E−05 | 0.000387482 | −0.160 | 0.160 | LOSS | 0.706 | 0.546 | | 37 |
| cg05226335 | 0.000307638 | 0.003033562 | −0.160 | 0.160 | LOSS | 0.637 | 0.477 | CTTN; CTTN; CTTN | 37 |
| cg24680439 | 0.000167527 | 0.00194356 | 0.160 | 0.160 | GAIN | 0.649 | 0.809 | LOC399829 | 37 |
| cg00497905 | 6.13E−06 | 0.000175468 | −0.160 | 0.160 | LOSS | 0.440 | 0.281 | MYO7A; MYO7A | 37 |
| cg23752752 | 5.49E−08 | 6.50E−06 | 0.160 | 0.160 | GAIN | 0.395 | 0.555 | FOXK1 | 37 |
| cg11210343 | 2.69E−11 | 1.11E−07 | −0.159 | 0.159 | LOSS | 0.405 | 0.246 | METAP2 | 37 |
| cg07512361 | 7.67E−07 | 3.99E−05 | −0.159 | 0.159 | LOSS | 0.628 | 0.469 | SH2B2 | 37 |
| cg05351887 | 7.00E−05 | 0.001021959 | 0.159 | 0.159 | GAIN | 0.350 | 0.509 | | 37 |
| cg01119278 | 9.38E−06 | 0.000238239 | 0.159 | 0.159 | GAIN | 0.613 | 0.772 | DDO; DDO | 37 |
| cg19092981 | 0.000252271 | 0.00262715 | −0.159 | 0.159 | LOSS | 0.582 | 0.423 | TBX1; TBX1; TBX1 | 37 |
| cg04799958 | 4.33E−08 | 5.55E−06 | 0.159 | 0.159 | GAIN | 0.625 | 0.784 | KRT18; KRT8; KRT8; | 37 |
| | | | | | | | | KRT18; KRT8; KRT8 | |
| cg19566405 | 0.001018834 | 0.007271153 | 0.159 | 0.159 | GAIN | 0.205 | 0.364 | SLFN12 | 37 |
| cg26995224 | 3.39E−08 | 4.75E−06 | 0.159 | 0.159 | GAIN | 0.578 | 0.737 | KDM2B; KDM2B | 37 |
| cg22841667 | 2.48E−07 | 1.81E−05 | −0.159 | 0.159 | LOSS | 0.407 | 0.249 | RPL27A; SNORA3; | 37 |
| | | | | | | | | SNORA45 | |
| cg07816074 | 1.57E−08 | 2.89E−06 | 0.158 | 0.158 | GAIN | 0.367 | 0.525 | SH3TC1 | 37 |
| cg22992730 | 0.001156175 | 0.008041808 | 0.158 | 0.158 | GAIN | 0.494 | 0.652 | | 37 |
| cg05164926 | 5.34E−07 | 3.08E−05 | −0.158 | 0.158 | LOSS | 0.291 | 0.133 | KCTD11 | 37 |
| cg01287088 | 1.57E−08 | 2.89E−06 | −0.158 | 0.158 | LOSS | 0.668 | 0.510 | PFN3 | 37 |
| cg06430632 | 1.61E−05 | 0.000353081 | −0.158 | 0.158 | LOSS | 0.604 | 0.446 | SFT2D1 | 37 |
| cg21697381 | 0.00059748 | 0.004922032 | 0.157 | 0.157 | GAIN | 0.225 | 0.383 | SLFN12 | 37 |
| cg10885151 | 1.65E−07 | 1.36E−05 | 0.157 | 0.157 | GAIN | 0.176 | 0.333 | | 37 |
| cg11057824 | 3.16E−07 | 2.15E−05 | −0.157 | 0.157 | LOSS | 0.663 | 0.507 | C14orf182 | 37 |
| cg06314111 | 1.62E−09 | 8.07E−07 | −0.157 | 0.157 | LOSS | 0.765 | 0.608 | AGAP2; AGAP2; AGAP2- | 37 |
| | | | | | | | | AS1 | |
| cg00551910 | 2.03E−07 | 1.56E−05 | −0.157 | 0.157 | LOSS | 0.542 | 0.386 | CCDC177 | 37 |
| cg02784823 | 9.38E−06 | 0.000238239 | 0.157 | 0.157 | GAIN | 0.695 | 0.852 | LMTK3 | 37 |
| cg04220104 | 2.64E−08 | 4.05E−06 | 0.157 | 0.157 | GAIN | 0.408 | 0.565 | MIR548N; TTN- | 37 |
| | | | | | | | | AS1; TTN-AS1 | |
| cg11123440 | 0.002714796 | 0.014844937 | −0.156 | 0.156 | LOSS | 0.659 | 0.503 | C8orf49 | 37 |
| cg27246571 | 1.19E−08 | 2.44E−06 | −0.156 | 0.156 | LOSS | 0.768 | 0.612 | HAL; HAL; HAL | 37 |
| cg14851700 | 3.45E−05 | 0.000609982 | −0.156 | 0.156 | LOSS | 0.466 | 0.310 | GLUL; GLUL | 37 |
| cg05396897 | 4.39E−05 | 0.000728646 | −0.156 | 0.156 | LOSS | 0.390 | 0.234 | NLRP3; NLRP3; NLRP3; | 37 |
| | | | | | | | | NLRP3; NLRP3; | |
| | | | | | | | | NLRP3 | |
| cg00873601 | 5.49E−08 | 6.50E−06 | 0.156 | 0.156 | GAIN | 0.333 | 0.489 | | 37 |
| cg04863892 | 1.08E−07 | 1.02E−05 | 0.156 | 0.156 | GAIN | 0.680 | 0.835 | HOXA5; HOXA5; HOXA- | 37 |
| | | | | | | | | AS3 | |
| cg13904806 | 4.57E−06 | 0.00014225 | −0.156 | 0.156 | LOSS | 0.922 | 0.766 | SAMD11 | 37 |
| cg08610426 | 0.000109302 | 0.001420029 | 0.156 | 0.156 | GAIN | 0.469 | 0.624 | IZUMO1 | 37 |
| cg01837362 | 3.45E−05 | 0.000609982 | −0.156 | 0.156 | LOSS | 0.533 | 0.378 | | 37 |
| cg18282375 | 2.55E−10 | 2.92E−07 | −0.156 | 0.156 | LOSS | 0.586 | 0.430 | HSPA12B; HSPA12B | 37 |
| cg14920846 | 6.13E−06 | 0.000175468 | −0.155 | 0.155 | LOSS | 0.440 | 0.285 | NAV1 | 37 |
| cg09320662 | 2.03E−07 | 1.56E−05 | 0.155 | 0.155 | GAIN | 0.408 | 0.563 | LRCOL1 | 37 |
| cg03775991 | 2.03E−07 | 1.56E−05 | 0.155 | 0.155 | GAIN | 0.632 | 0.787 | | 37 |
| cg13750264 | 0.004580487 | 0.021752094 | −0.155 | 0.155 | LOSS | 0.600 | 0.445 | GPR123 | 37 |
| cg23188684 | 4.39E−05 | 0.000728646 | 0.155 | 0.155 | GAIN | 0.466 | 0.621 | DOC2GP | 37 |
| cg01331992 | 2.62E−09 | 9.86E−07 | −0.155 | 0.155 | LOSS | 0.551 | 0.396 | RPS6 | 37 |
| cg19827875 | 3.39E−08 | 4.75E−06 | −0.155 | 0.155 | LOSS | 0.932 | 0.777 | NAV1 | 37 |
| cg04865726 | 7.67E−07 | 3.99E−05 | −0.155 | 0.155 | LOSS | 0.347 | 0.192 | | 37 |
| cg14898243 | 6.91E−08 | 7.57E−06 | −0.155 | 0.155 | LOSS | 0.795 | 0.641 | SRGN; SRGN | 37 |
| cg06576532 | 0.000185879 | 0.002097946 | −0.155 | 0.155 | LOSS | 0.565 | 0.411 | | 37 |
| cg00011924 | 2.28E−05 | 0.000463162 | −0.155 | 0.155 | LOSS | 0.522 | 0.367 | RNF222; RNF222 | 37 |
| cg03463411 | 0.001430091 | 0.009288043 | 0.155 | 0.155 | GAIN | 0.400 | 0.555 | PRDM8; PRDM8 | 37 |
| cg05836043 | 5.34E−07 | 3.08E−05 | −0.155 | 0.155 | LOSS | 0.659 | 0.505 | LAMA1 | 37 |
| cg13541527 | 6.73E−09 | 1.68E−06 | −0.154 | 0.154 | LOSS | 0.427 | 0.272 | C6orf48; C6orf48; | 37 |
| | | | | | | | | SNORD52 | |
| cg03415617 | 1.08E−05 | 0.000263209 | −0.154 | 0.154 | LOSS | 0.647 | 0.493 | | 37 |
| cg22970003 | 0.000121826 | 0.001535447 | 0.154 | 0.154 | GAIN | 0.263 | 0.417 | PTPRN2; PTPRN2; | 37 |
| | | | | | | | | PTPRN2 | |
| cg01413354 | 0.000339214 | 0.003261865 | 0.154 | 0.154 | GAIN | 0.418 | 0.572 | RALGDS | 37 |
| cg17624673 | 1.65E−07 | 1.36E−05 | −0.154 | 0.154 | LOSS | 0.659 | 0.505 | PCDHB13 | 37 |
| cg10323490 | 1.19E−08 | 2.44E−06 | −0.154 | 0.154 | LOSS | 0.762 | 0.609 | THNSL2; THNSL2 | 37 |
| cg26056277 | 0.004580487 | 0.021752094 | −0.154 | 0.154 | LOSS | 0.605 | 0.451 | SCN1A | 37 |
| cg07637837 | 3.05E−05 | 0.000559351 | −0.153 | 0.153 | LOSS | 0.671 | 0.517 | MBP; MBP | 37 |
| cg17187762 | 0.00041121 | 0.003747585 | 0.153 | 0.153 | GAIN | 0.577 | 0.731 | | 37 |
| cg09748975 | 6.41E−07 | 3.50E−05 | −0.153 | 0.153 | LOSS | 0.411 | 0.258 | MSX1 | 37 |
| cg09652312 | 4.33E−08 | 5.55E−06 | 0.153 | 0.153 | GAIN | 0.542 | 0.695 | | 37 |
| cg19937979 | 0.000339214 | 0.003261865 | −0.153 | 0.153 | LOSS | 0.535 | 0.382 | CCDC177 | 37 |
| cg22410743 | 2.11E−06 | 8.17E−05 | −0.153 | 0.153 | LOSS | 0.778 | 0.626 | IQCH; IQCH; IQCH; | 37 |
| | | | | | | | | IQCH; IQCH | |
| cg00344445 | 1.08E−05 | 0.000263209 | −0.153 | 0.153 | LOSS | 0.783 | 0.630 | FLI1; FLI1; FLI1; FLI1 | 37 |
| cg14573099 | 9.00E−10 | 5.32E−07 | −0.152 | 0.152 | LOSS | 0.742 | 0.590 | TBC1D8 | 37 |
| cg16322792 | 0.009765492 | 0.037891033 | −0.152 | 0.152 | LOSS | 0.488 | 0.335 | ZNF697 | 37 |
| cg16875104 | 4.33E−08 | 5.55E−06 | −0.152 | 0.152 | LOSS | 0.454 | 0.302 | GARS | 37 |
| cg10431713 | 0.00041121 | 0.003747585 | 0.152 | 0.152 | GAIN | 0.143 | 0.294 | SLFN12 | 37 |
| cg26679004 | 2.48E−07 | 1.81E−05 | 0.152 | 0.152 | GAIN | 0.298 | 0.450 | GRID1 | 37 |
| cg14371731 | 2.03E−07 | 1.56E−05 | 0.152 | 0.152 | GAIN | 0.026 | 0.178 | ZMIZ1 | 37 |
| cg10213542 | 2.90E−06 | 0.000102762 | 0.152 | 0.152 | GAIN | 0.374 | 0.525 | ADAMTS2; ADAMTS2 | 37 |
| cg06473363 | 1.19E−08 | 2.44E−06 | −0.151 | 0.151 | LOSS | 0.770 | 0.618 | GPANK1; GPANK1; | 37 |
| | | | | | | | | GPANK1; GPANK1; | |
| | | | | | | | | GPANK1 | |
| cg02734505 | 1.08E−07 | 1.02E−05 | −0.151 | 0.151 | LOSS | 0.424 | 0.273 | ZNF385A; ZNF385A; | 37 |
| | | | | | | | | ZNF385A | |
| cg15233961 | 1.57E−08 | 2.89E−06 | 0.151 | 0.151 | GAIN | 0.415 | 0.566 | | 37 |
| cg06470855 | 1.08E−05 | 0.000263209 | 0.151 | 0.151 | GAIN | 0.669 | 0.820 | | 37 |
| cg26135325 | 1.29E−06 | 5.75E−05 | −0.151 | 0.151 | LOSS | 0.351 | 0.200 | LCE3A | 37 |
| cg24049888 | 1.41E−05 | 0.000320585 | 0.151 | 0.151 | GAIN | 0.319 | 0.470 | POU2AF1; POU2AF1 | 37 |
| cg20088245 | 0.00041121 | 0.003747585 | 0.151 | 0.151 | GAIN | 0.565 | 0.716 | | 37 |
| cg03128011 | 0.000185879 | 0.002097946 | 0.151 | 0.151 | GAIN | 0.585 | 0.736 | | 37 |
| cg24852442 | 6.41E−07 | 3.50E−05 | −0.151 | 0.151 | LOSS | 0.417 | 0.266 | MYO7A; MYO7A | 37 |
| cg19276111 | 4.33E−08 | 5.55E−06 | −0.150 | 0.150 | LOSS | 0.358 | 0.208 | CNR2 | 37 |
| cg00693004 | 0.000514392 | 0.004451981 | −0.150 | 0.150 | LOSS | 0.676 | 0.525 | NMT1 | 37 |
| cg16194588 | 1.52E−06 | 6.46E−05 | 0.150 | 0.150 | GAIN | 0.669 | 0.819 | LMTK3 | 37 |
| cg08551532 | 0.000167527 | 0.00194356 | −0.150 | 0.150 | LOSS | 0.276 | 0.126 | DLL3; DLL3 | 37 |
| cg16481961 | 0.000121826 | 0.001535447 | 0.150 | 0.150 | GAIN | 0.125 | 0.275 | MIR596 | 37 |
| cg20907614 | 0.000109302 | 0.001420029 | −0.150 | 0.150 | LOSS | 0.689 | 0.538 | | 37 |
| cg20806296 | 3.64E−09 | 1.18E−06 | −0.150 | 0.150 | LOSS | 0.664 | 0.514 | | 37 |

| | | Genomic | | | Relation to |
|---|---|---|---|---|---|
| | | Coordinate | | | transcription |
| Illumina ID | Chromosome | (NCBI, hg19) | Strand | Relation_to_UCSC_CpG_Island | start site (TSS) |
| cg22987448 | 19 | 8591364 | F | Island | MYO1F(body) |
| cg15254671 | 19 | 8591513 | F | Island | MYO1F(body) |
| cg05857996 | 20 | 2675418 | F | S_Shore | EBF4(body) |
| cg08283130 | 19 | 8591776 | R | Island | MYO1F(body) |
| cg01178624 | 11 | 65360327 | R | Island | KCNK7(body) |
| cg00274965 | 21 | 34405681 | F | Island | |
| cg09232555 | 8 | 11619866 | R | | C8orf49(body) |
| cg22568423 | 19 | 8590567 | F | N_Shore | MYO1F(body) |
| cg08818610 | 6 | 24910720 | F | Island | FAM65B(body) |
| cg16370398 | 12 | 54448913 | F | S_Shore | HOXC4(body) |
| cg15954353 | 17 | 5403337 | F | Island | LOC728392(body) |
| cg05825244 | 20 | 2730488 | F | Island | EBF4(body) |
| cg09226051 | 1 | 247611502 | R | N_Shelf | NLRP3(body) |
| cg21637392 | 7 | 5735123 | R | | RNF216(body) |
| cg14172108 | 21 | 34405553 | R | N_Shore | |
| cg11532431 | 7 | 27169674 | F | Island | HOXA4(body) |
| cg20543544 | 10 | 81003657 | R | Island | ZMIZ1(body) |
| cg05491854 | 6 | 24910562 | F | N_Shore | FAM65B(body) |
| cg08255475 | 16 | 88871329 | R | N_Shore | CDT1(body) |
| cg08425810 | 12 | 58132558 | R | Island | AGAP2(body); AGAP2 |
| | | | | | (tss1500) |
| cg22997113 | 7 | 27170241 | R | Island | HOXA4(body) |
| cg15454820 | 10 | 96990858 | F | | |
| cg14911689 | 12 | 739980 | F | | NINJ2(body) |
| cg25308803 | 2 | 109746735 | F | Island | SH3RF3(body); SH3RF3- |
| | | | | | AS1(tss200) |
| cg10785373 | 7 | 4456119 | F | | |
| cg23387569 | 12 | 58120011 | R | Island | AGAP2(body); AGAP2- |
| | | | | | AS1(tss200) |
| cg00313914 | 1 | 201618901 | R | Island | NAV1(body) |
| cg03846641 | 2 | 109746751 | F | Island | SH3RF3(body); SH3RF3- |
| | | | | | AS1(tss200) |
| cg14099457 | 3 | 49170794 | R | | LAMB2(tss200) |
| cg19738980 | 18 | 7011463 | F | Island | LAMA1(body) |
| cg19142026 | 7 | 27170394 | R | Island | HOXA4(body) |
| cg09549073 | 7 | 27183274 | F | Island | HOXA- |
| | | | | | AS3(body); HOXA5 |
| | | | | | (body) |
| cg04287574 | 1 | 201619622 | R | Island | NAV1(body) |
| cg03269218 | 10 | 96990700 | F | | |
| cg05905531 | 19 | 8591721 | F | Island | MYO1F(body) |
| cg12474798 | 10 | 64565772 | R | Island | ADO(body) |
| cg20225999 | 2 | 218843435 | F | N_Shore | |
| cg24690094 | 11 | 67383802 | R | Island | DOC2GP(tss1500) |
| cg02224314 | 14 | 99641151 | R | Island | BCL11B(body) |
| cg18025886 | 3 | 196750939 | R | N_Shelf | MFI2(body) |
| cg03146625 | 12 | 54448729 | F | S_Shore | HOXC4(body) |
| cg21429551 | 7 | 30635762 | F | S_Shore | GARS(body) |
| cg03455316 | 15 | 62516405 | R | Island | |
| cg06663305 | 17 | 8095813 | R | S_Shelf | |
| cg09817024 | 8 | 11471395 | R | S_Shore | |
| cg09214243 | 15 | 29968124 | R | S_Shore | |
| cg01246520 | 17 | 17644344 | F | | RAI1(body) |
| cg26404511 | 1 | 24229575 | R | S_Shore | CNR2(body) |
| cg15795305 | 10 | 102381344 | R | | |
| cg14018024 | 9 | 133908909 | R | N_Shelf | LAMC3(body) |
| cg20704450 | 1 | 228658371 | F | N_Shore | |
| cg14759565 | 11 | 65360123 | R | Island | |
| cg24263062 | 20 | 2730191 | F | Island | EBF4(body) |
| cg26654770 | 12 | 740100 | F | | NINJ2(body) |
| cg12128839 | 7 | 27183436 | R | Island | HOXA- |
| | | | | | AS3(body); HOXA5 |
| | | | | | (tss200) |
| cg04517524 | 14 | 94405342 | F | Island | ASB2(body) |
| cg11015251 | 7 | 27170554 | F | Island | HOXA4(tss200) |
| cg11693285 | 10 | 131927345 | R | Island | |
| cg24217894 | 12 | 58120635 | F | Island | AGAP2(body); AGAP2- |
| | | | | | AS1(body) |
| cg24680632 | 12 | 116044032 | R | | |
| cg08347626 | 5 | 1850140 | F | N_Shore | |
| cg23901918 | 10 | 105420747 | F | Island | SH3PXD2A(body) |
| cg06847624 | 5 | 176827671 | R | Island | PFN3(tss200) |
| cg03068497 | 7 | 30635838 | R | S_Shore | GARS(body) |
| cg00815832 | 1 | 228658973 | F | Island | |
| cg27403406 | 20 | 48325721 | R | N_Shelf | B4GALT5(body) |
| cg01994308 | 8 | 57122990 | F | N_Shore | CHCHD7(tss1500); |
| | | | | | PLAG1(body) |
| cg05991492 | 16 | 3988700 | R | N_Shore | |
| cg13379325 | 20 | 62052259 | R | Island | KCNQ2(body) |
| cg23549902 | 7 | 5184155 | F | Island | ZNF890P(body) |
| cg00401101 | 5 | 16509323 | F | | FAM134B(body); FAM134B |
| | | | | | (tss1500) |
| cg14845962 | 12 | 58120237 | R | Island | AGAP2(body); AGAP2- |
| | | | | | AS1(body) |
| cg23669081 | 17 | 46685353 | R | Island | HOXB7(body) |
| cg16651126 | 7 | 27170552 | F | Island | HOXA4(tss200) |
| cg11336382 | 1 | 228658646 | R | N_Shore | |
| cg00130223 | 16 | 33070551 | F | Island | |
| cg06904356 | 5 | 1849983 | R | N_Shore | |
| cg01238044 | 22 | 24384105 | F | N_Shore | GSTT1(body) |
| cg07211044 | 8 | 60032983 | R | S_Shore | TOX(tss1500) |
| cg24927841 | 8 | 129702875 | R | | |
| cg10146935 | 1 | 871308 | R | Island | SAMD11(body) |
| cg19579217 | 6 | 10720630 | R | N_Shelf | |
| cg25910261 | 7 | 157405965 | F | Island | PTPRN2(body) |
| cg17740434 | 2 | 179388064 | F | | MIR548N(body); TTN- |
| | | | | | AS1(body) |
| cg02919082 | 6 | 32605694 | F | | HLA-DQA1(body) |
| cg02616966 | 3 | 182817190 | F | Island | MCCC1(body) |
| cg11510586 | 9 | 72027409 | R | Island | |
| cg20100745 | 8 | 134307728 | F | N_Shore | NDRG1(body) |
| cg02715602 | 19 | 4544446 | F | Island | SEMA6B(body) |
| cg07599786 | 1 | 201618654 | F | Island | NAV1(body) |
| cg16423910 | 19 | 49843627 | F | Island | CD37(body) |
| cg08911368 | 8 | 11471085 | R | Island | |
| cg03930209 | 7 | 156735466 | R | Island | |
| cg16440561 | 2 | 220312854 | F | Island | SPEG(body) |
| cg23489137 | 2 | 161290449 | R | | RBMS1(body) |
| cg02639108 | 2 | 242711009 | R | Island | |
| cg11410718 | 7 | 27170412 | R | Island | HOXA4(tss200) |
| cg05463589 | 16 | 88706426 | F | Island | IL17C(body) |
| cg16823042 | 12 | 58119992 | R | Island | AGAP2(body); AGAP2- |
| | | | | | AS1(tss200) |
| cg03613822 | 17 | 7115140 | R | N_Shelf | DLG4(body) |
| cg24652615 | 6 | 44243304 | R | Island | TMEM151B(body) |
| cg16565409 | 17 | 27048223 | R | S_Shore | RPL23A(body); SNOARD4A |
| | | | | | (tss1500) |
| cg13518079 | 20 | 2675072 | R | S_Shore | EBF4(body) |
| cg02892925 | 8 | 60032926 | R | S_Shore | TOX(tss1500) |
| cg17569124 | 7 | 27183643 | R | Island | HOXA- |
| | | | | | AS3(body); HOXA5 |
| | | | | | (tss1500) |
| cg19196401 | 6 | 110721138 | R | Island | DDO(body) |
| cg13068698 | 7 | 35078082 | F | S_Shore | DPY19L1(tss1500) |
| cg07317062 | 7 | 27170388 | R | Island | HOXA4(body) |
| cg10648815 | 19 | 55013549 | R | | LAIR2(tss1500) |
| cg03651054 | 13 | 50194643 | F | | |
| cg16814680 | 8 | 91681699 | F | | |
| cg12097883 | 17 | 62774939 | R | Island | LOC146880(body) |
| cg23061725 | 2 | 202126379 | R | | CASP8(body) |
| cg14359292 | 7 | 27170892 | F | S_Shore | HOXA4(tss1500) |
| cg18424841 | 20 | 61315444 | F | Island | |
| cg04991337 | 3 | 182817223 | F | Island | MCCC1(body) |
| cg02439789 | 1 | 871441 | R | Island | SAMD11(body) |
| cg01948217 | 20 | 36932385 | F | | BPI(tss200) |
| cg12748890 | 1 | 27676205 | F | Island | SYTL1(body) |
| cg11969813 | 17 | 79816559 | R | N_Shore | P4HB(body) |
| cg18977541 | 10 | 102381532 | R | | |
| cg16734913 | 11 | 55681277 | F | | OR5W2(body) |
| cg22220710 | 18 | 7011217 | F | N_Shore | LAMA1(body) |
| cg03604073 | 19 | 47507409 | R | Island | ARHGAP35(body) |
| cg25513090 | 7 | 6488668 | F | S_Shore | DAGLB(tss1500) |
| cg26823666 | 1 | 228658397 | F | N_Shore | |
| cg20016023 | 10 | 99160130 | R | N_Shore | RRP12(body) |
| cg24753998 | 21 | 30452964 | R | | MAP3K7CL(body) |
| cg26371957 | 12 | 739280 | F | | NINJ2(body) |
| cg25307665 | 7 | 27183694 | R | Island | HOXA- |
| | | | | | AS3(body); HOXA5 |
| | | | | | (tss1500) |
| cg20978937 | 14 | 105399321 | R | Island | PLD4(body) |
| cg08234664 | 3 | 49170668 | F | | LAMB2(tss200) |
| cg13619522 | 15 | 75095171 | R | N_Shore | CSK(body) |
| cg20698421 | 2 | 65217623 | F | S_Shore | SLC1A4(body) |
| cg11511175 | 12 | 58119979 | R | Island | AGAP2(body); AGAP2- |
| | | | | | AS1(tss200) |
| cg27001715 | 6 | 150329845 | R | S_Shelf | |
| cg19916659 | 2 | 179387931 | R | | MIR548N(body); TTN- |
| | | | | | AS1(body) |
| cg07616871 | 2 | 218843504 | F | Island | |
| cg21476494 | 12 | 116043958 | R | | |
| cg20007021 | 14 | 24780404 | F | Island | CIDEB(body); LTB4R |
| | | | | | (tss1500); LTB4R2 |
| | | | | | (body) |
| cg10044179 | 21 | 15352983 | F | S_Shore | ANKRD20A11P(tss |
| | | | | | 1500) |
| cg12176783 | 20 | 62694000 | F | Island | TCEA2(body); TCEA2 |
| | | | | | (tss200) |
| cg02954987 | 3 | 49170599 | F | | LAMB2(body) |
| cg21570209 | 19 | 46367987 | R | S_Shore | FOXA3(body); SYMPK |
| | | | | | (tss1500) |
| cg24937727 | 19 | 11517079 | F | Island | RGL3(body) |
| cg22582187 | 10 | 63394414 | F | | |
| cg03043406 | 1 | 45242356 | R | S_Shore | RPS8(body); SNORD38A |
| | | | | | (tss1500) |
| cg09636302 | 12 | 96389483 | F | Island | HAL(body) |
| cg24550112 | 1 | 14027521 | R | S_Shore | PRDM2(body) |
| cg17900689 | 17 | 4649262 | F | | ZMYND15(body) |
| cg24524285 | 11 | 64405919 | R | Island | NRXN2(body) |
| cg17655970 | 13 | 112985463 | R | Island | |
| cg24194775 | 9 | 35791475 | R | N_Shore | NPR2(tss1500) |
| cg04387835 | 17 | 4649076 | F | | ZMYND15(body) |
| cg26411441 | 20 | 3733040 | R | S_Shore | HSPA12B(body) |
| cg24517467 | 7 | 155284331 | R | Island | |
| cg08657492 | 7 | 27170832 | F | S_Shore | HOXA4(tss1500) |
| cg17431280 | 19 | 47507461 | R | Island | ARHGAP35(body) |
| cg20748533 | 19 | 51189975 | R | Island | SHANK1(body) |
| cg21111256 | 19 | 41386507 | R | Island | CYP2A7(body) |
| cg06768599 | 14 | 24785488 | R | Island | LTB4R(body) |
| cg18090145 | 6 | 67741714 | F | | |
| cg00343839 | 17 | 5403516 | F | Island | LOC728392(body) |
| cg00290607 | 11 | 67383545 | R | Island | DOC2GP(tss1500) |
| cg00011856 | 2 | 217560946 | R | S_Shore | IGFBP5(tss1500) |
| cg24940967 | 3 | 45837197 | R | N_Shore | SLC6A20(body) |
| cg15265092 | 6 | 34723499 | F | N_Shore | SNRPC(tss1500) |
| cg25288140 | 17 | 41278341 | F | Island | BRCA1(tss1500); NBR2 |
| | | | | | (body) |
| cg19786602 | 17 | 7966326 | F | | |
| cg13614409 | 13 | 31506752 | F | | TEX26(tss200); TEX26- |
| | | | | | AS1(tss200) |
| cg27539527 | 7 | 156735656 | R | Island | |
| cg01834979 | 12 | 58119918 | F | Island | AGAP2(body); AGAP2- |
| | | | | | AS1(tss200) |
| cg23060513 | 19 | 13041124 | F | N_Shelf | FARSA(body) |
| cg08739651 | 10 | 51784888 | R | S_Shore | FLJ31813(body) |
| cg08355456 | 11 | 67383691 | R | Island | DOC2GP(tss1500) |
| cg18322589 | 10 | 123909456 | F | | TACC2(body) |
| cg24576298 | 7 | 108137995 | F | | PNPLA8(body) |
| cg06015422 | 8 | 70907139 | F | | |
| cg22259797 | 11 | 118986860 | F | | C2CD2L(body) |
| cg18587137 | 14 | 103593503 | R | Island | TNFAIP2(body) |
| cg18784409 | 11 | 67868331 | F | | CHKA(body) |
| cg13759905 | 2 | 233741920 | F | S_Shore | |
| cg09284949 | 19 | 51190179 | R | S_Shore | SHANK1(body) |
| cg03701930 | 10 | 1981436 | F | | |
| cg03691722 | 18 | 7011268 | R | Island | LAMA1(body) |
| cg27466845 | 11 | 64397734 | F | Island | NRXN2(body) |
| cg16915863 | 12 | 54523294 | F | S_Shelf | LOC400043(body) |
| cg12133451 | 1 | 227746453 | F | Island | |
| cg06137123 | 11 | 129444480 | R | | |
| cg10501093 | 14 | 103593520 | R | Island | TNFAIP2(body) |
| cg08801017 | 13 | 19918525 | F | N_Shore | ANKRD26P3(body); |
| | | | | | LINC00421(tss1500) |
| cg16312514 | 11 | 70650521 | R | | SHANK2(body) |
| cg02666610 | 11 | 67499431 | R | | |
| cg19321684 | 6 | 32159933 | R | N_Shelf | GPSM3(body) |
| cg05076221 | 7 | 27182637 | F | Island | HOXA- |
| | | | | | AS3(body);HOXA5 |
| | | | | | (body) |
| cg23502204 | 11 | 87905295 | R | N_Shelf | RAB38(body) |
| cg20299697 | 3 | 138069423 | F | S_Shore | MRAS(body) |
| cg07040013 | 10 | 132099553 | F | | |
| cg07509935 | 14 | 24780167 | F | Island | CIDEB(body); LTB4R |
| | | | | | (tss1500); LTB4R2 |
| | | | | | (body) |
| cg01231141 | 5 | 178692691 | F | | ADAMTS2(body) |
| cg22127848 | 17 | 64295986 | R | N_Shelf | |
| cg04015962 | 1 | 10949192 | F | | |
| cg05226335 | 11 | 70253499 | R | N_Shelf | CTTN(body) |
| cg24680439 | 10 | 134778467 | F | N_Shore | LOC399829(tss1500) |
| cg00497905 | 11 | 76903183 | F | | MYO7A(body) |
| cg23752752 | 7 | 4778908 | R | | FOXK1(body) |
| cg11210343 | 12 | 95869153 | F | S_Shore | METAP2(body) |
| cg07512361 | 7 | 101944430 | R | Island | SH2B2(body) |
| cg05351887 | 16 | 3988869 | R | N_Shore | |
| cg01119278 | 6 | 110721349 | F | Island | DDO(body) |
| cg19092981 | 22 | 19751654 | F | Island | TBX1(body) |
| cg04799958 | 12 | 53343849 | F | S_Shore | KRT18(body); KRT8 |
| | | | | | (tss200) |
| cg19566405 | 17 | 33759965 | F | | SLFN12(tss1500) |
| cg26995224 | 12 | 121974146 | R | N_Shore | KDM2B(body) |
| cg22841667 | 11 | 8705620 | F | S_Shore | RPL27A(body); SNORA3 |
| | | | | | (tss200); SNORA45 |
| | | | | | (tss1500) |
| cg07816074 | 4 | 8201560 | F | | SH3TC1(body) |
| cg22992730 | 19 | 4784940 | F | N_Shore | |
| cg05164926 | 17 | 7255624 | F | Island | KCTD11(body) |
| cg01287088 | 5 | 176827392 | F | Island | PFN3(body) |
| cg06430632 | 6 | 166746926 | F | | SFT2D1(body) |
| cg21697381 | 17 | 33759957 | R | | SLFN12(tss1500) |
| cg10885151 | 13 | 24270087 | F | Island | |
| cg11057824 | 14 | 50471938 | F | S_Shore | C14orf182(body) |
| cg06314111 | 12 | 58119915 | F | Island | AGAP2(body); AGAP2- |
| | | | | | AS1(tss200) |
| cg00551910 | 14 | 70037973 | R | N_Shore | CCDC177(body) |
| cg02784823 | 19 | 49000897 | F | Island | LMTK3(body) |
| cg04220104 | 2 | 179387853 | F | | MIR548N(body); TTN- |
| | | | | | AS1(body); TTN- |
| | | | | | AS1(tss200) |
| cg11123440 | 8 | 11619852 | R | | C8orf49(body) |
| cg27246571 | 12 | 96389588 | R | Island | HAL(body) |
| cg14851700 | 1 | 182362230 | F | S_Shore | GLUL(tss1500) |
| cg05396897 | 1 | 247611448 | R | N_Shelf | NLRP3(body) |
| cg00873601 | 12 | 116044025 | R | | |
| cg04863892 | 7 | 27183375 | R | Island | HOXA- |
| | | | | | AS3(body); HOXA5 |
| | | | | | (tss200) |
| cg13904806 | 1 | 874697 | F | N_Shore | SAMD11(body) |
| cg08610426 | 19 | 49249123 | F | | IZUMO1(body) |
| cg01837362 | 12 | 34492938 | R | N_Shore | |
| cg18282375 | 20 | 3732920 | F | Island | HSPA12B(body) |
| cg14920846 | 1 | 201618209 | R | Island | NAV1(body) |
| cg09320662 | 12 | 133180698 | F | S_Shore | LRCOL1(body) |
| cg03775991 | 6 | 170589530 | R | Island | |
| cg13750264 | 10 | 134910540 | F | N_Shore | GPR123(body) |
| cg23188684 | 11 | 67383651 | F | Island | DOC2GP(tss1500) |
| cg01331992 | 9 | 19379118 | R | N_Shore | RPS6(body) |
| cg19827875 | 1 | 201618284 | F | Island | NAV1(body) |
| cg04865726 | 1 | 1365911 | R | S_Shelf | |
| cg14898243 | 10 | 70863693 | R | | SRGN(body) |
| cg06576532 | 10 | 3282437 | F | | |
| cg00011924 | 17 | 8301192 | R | | RNF222(tss200) |
| cg03463411 | 4 | 81118188 | F | Island | PRDM8(body); PRDM8 |
| | | | | | (tss1500) |
| cg05836043 | 18 | 7011388 | F | Island | LAMA1(body) |
| cg13541527 | 6 | 31804078 | F | S_Shore | C6orf48(body); SNORD52 |
| | | | | | (tss1500) |
| cg03415617 | 16 | 34726856 | F | | |
| cg22970003 | 7 | 157406032 | R | Island | PTPRN2(body) |
| cg01413354 | 9 | 136017755 | R | N_Shore | RALGDS(body) |
| cg17624673 | 5 | 140596187 | R | S_Shore | PCDHB13(body) |
| cg10323490 | 2 | 88469007 | F | N_Shore | THNSL2(tss1500) |
| cg26056277 | 2 | 166982925 | F | | SCN1A(body) |
| cg07637837 | 18 | 74824154 | F | Island | MBP(body) |
| cg17187762 | 22 | 28070120 | R | N_Shelf | |
| cg09748975 | 4 | 4864532 | F | Island | MSX1(body) |
| cg09652312 | 7 | 155284062 | R | Island | |
| cg19937979 | 14 | 70039915 | F | Island | CCDC177(body) |
| cg22410743 | 15 | 67574897 | R | | IQCH(body) |
| cg00344445 | 11 | 128647107 | R | | FLI1(body) |
| cg14573099 | 2 | 101761014 | F | | TBC1D8(body) |
| cg16322792 | 1 | 120165303 | F | Island | ZNF697(body) |
| cg16875104 | 7 | 30635889 | R | S_Shore | GARS(body) |
| cg10431713 | 17 | 33760230 | F | | SLFN12(tss1500) |
| cg26679004 | 10 | 88023135 | R | Island | GRID1(body) |
| cg14371731 | 10 | 81003175 | R | Island | ZMIZ1(body) |
| cg10213542 | 5 | 178692728 | F | | ADAMTS2(body) |
| cg06473363 | 6 | 31631797 | F | N_Shore | GPANK1(body) |
| cg02734505 | 12 | 54763081 | R | Island | ZNF385A(body) |
| cg15233961 | 10 | 96990543 | R | | |
| cg06470855 | 13 | 112997365 | R | Island | |
| cg26135325 | 1 | 152595322 | R | | LCE3A(body) |
| cg24049888 | 11 | 111250129 | F | | POU2AF1(body) |
| cg20088245 | 8 | 1321375 | R | Island | |
| cg03128011 | 8 | 1321333 | R | Island | |
| cg24852442 | 11 | 76903134 | R | | MYO7A(body) |
| cg19276111 | 1 | 24229232 | R | Island | CNR2(body) |
| cg00693004 | 17 | 43151433 | F | | NMT1(body) |
| cg16194588 | 19 | 49002477 | F | Island | LMTK3(body) |
| cg08551532 | 19 | 39998270 | F | Island | DLL3(body) |
| cg16481961 | 8 | 1765421 | F | S_Shore | MIR596(body) |
| cg20907614 | 8 | 29914963 | F | | |
| cg20806296 | 2 | 138582049 | F | | |

TABLE 10
Cross-validation results for different effect-size (absolute delta beta, |Δβ|) thresholds at p-value ≦0.05. Shown are the specificity (Spec)
and sensitivity (Sens) of the LOO procedure. Specificity is for 1056 normal blood samples derived from GEO (Spec GEO). The total number
of significant sites (CGs) in the resulting “Kabuki signature” set, the gene names (Names) and their total number (Genes)
corresponding to the significant sites are provided. One optimal combination was selected to be p-value ≦0.05 and |Δβ|>15%
(highlighted in bold). The p-values are corrected for multiple testing (Benjamini-Hochberg correction).

| | p-value ≦0.05 | | | | | |
|---|---|---|---|---|---|---|
| | | | Spec | | | |
| \|Δβ\| | Spec | Sens | (GEO) | CGs | Names | Genes |
| 5% | 100.0% | 90.9% | 99.9% | 13595 | Not shown | 6479 |
| 10% | 100.0% | 90.9% | 100.0% | 1941 | Not shown | 1093 |
| 15% | 100.0% | 100.0% | 100.0% | 287 | ADAMTS2; ADO; AGAP2; AGAP2-AS1; ANKRD20A11P; ANKRD26P3; ARHGAP35; | 162 |
| | | | | | ASB2; B4GALT5; BCL11B; BPI; BRCA1; C14orf182; C2CD2L; C6orf48; C8orf49; CASP8; | |
| | | | | | CCDC177; CD37; CDT1; CHCHD7; CHKA; CIDEB; CNR2; CSK; CTTN; CYP2A7; DAGLB; | |
| | | | | | DDO; DLG4; DLL3; DOC2GP; DPY19L1; EBF4; FAM134B; FAM65B; FARSA; FLI1; | |
| | | | | | FLJ31813; FOXA3; FOXK1; GARS; GLUL; GPANK1; GPR123; GPSM3; GRID1; GSTT1; | |
| | | | | | HAL; HLA-DQA1; HOXA-AS3; HOXA4; HOXA5; HOXB7; HOXC4; HSPA12B; IGFBP5; | |
| | | | | | IL17C; IQCH; IZUMO1; KCNK7; KCNQ2; KCTD11; KDM2B; KRT18; KRT8; LAIR2; | |
| | | | | | LAMA1; LAMB2; LAMC3; LCE3A; LINC00421; LMTK3; LOC146880; LOC399829; | |
| | | | | | LOC400043; LOC728392; LRCOL1; LTB4R; LTB4R2; MAP3K7CL; MBP; MCCC1; | |
| | | | | | METAP2; MFI2; MIR548N; MIR596; MRAS; MSX1; MYO1F; MYO7A; NAV1; NBR2; | |
| | | | | | NDRG1; NINJ2; NLRP3; NMT1; NPR2; NRXN2; OR5W2; P4HB; PCDHB13; PFN3; | |
| | | | | | PLAG1; PLD4; PNPLA8; POU2AF1; PRDM2; PRDM8; PTPRN2; RAB38; RAI1; | |
| | | | | | RALGDS; RBMS1; RGL3; RNF216; RNF222; RPL23A; RPL27A; RPS6; RPS8; RRP12; | |
| | | | | | SAMD11; SCN1A; SEMA6B; SFT2D1; SH2B2; SH3PXD2A; SH3RF3; SH3RF3-AS1; | |
| | | | | | SH3TC1; SHANK1; SHANK2; SLC1A4; SLC6A20; SLFN12; SNORA3; SNORA45; | |
| | | | | | SNORD38A; SNORD4A; SNORD52; SNRPC; SPEG; SRGN; SYMPK; SYTL1; TACC2; | |
| | | | | | TBC1D8; TBX1; TCEA2; TEX26; TEX26-AS1; THNSL2; TMEM151B; TNFAIP2; TOX; | |
| | | | | | TTN-AS1; ZMIZ1; ZMYND15; ZNF385A; ZNF697; ZNF890P | |
| 20% | 100.0% | 100.0% | 100.0% | 46 | ADO; AGAP2; AGAP2-AS1; BCL11B; C8orf49; CDT1; DOC2GP; EBF4; | 27 |
| | | | | | FAM65B; GARS; HOXA- AS3; HOXA4; HOXA5; HOXC4; KCNK7; LAMA1; LAMB2; | |
| | | | | | LOC728392; MFI2; MYO1F; NAV1; NINJ2; NLRP3; RNF216; SH3RF3; SH3RF3-AS1; | |
| | | | | | ZMIZ1 | |
| 25% | 100.0% | 54.5% | 99.1% | 10 | C8or149; EBF4; FAM65B; HOXC4; KCNK7; MYO1F | 6 |

TABLE 11
Cross-validation results for different effect-size (absolute delta beta, |Δβ|) thresholds at p-value ≦0.01. Shown are the specificity
(Spec) and sensitivity (Sens) of the LOO procedure. Specificity is for 1056 normal blood samples derived from GEO (Spec GEO). The total
number of significant sites (CGs) in the resulting “Kabuki signature” set, the gene names (Names) and their total number (Genes) corresponding to
the significant sites are provided. The p-values are corrected for multiple testing (Benjamini-Hochberg correction).

| | p-value ≦0.01 | | | | | |
|---|---|---|---|---|---|---|
| | | | Spec | | | |
| \|Δβ\| | Spec | Sens | (GEO) | CGs | Names | Genes |
| 5% | 100.0% | 90.9% | 99.9% | 9993 | Not shown | 5247 |
| 10% | 100.0% | 90.9% | 100.0% | 1704 | Not shown | 970 |
| 15% | 100.0% | 100.0% | 100.0% | 272 | ADAMTS2; ADO; AGAP2; AGAP2- | 153 |
| | | | | | AS1; ANKRD26P3; ARHGAP35; ASB2; B4GALT5; BCL11B; BPI; BRCA1; C14orf182; | |
| | | | | | C6orf48; C8orf49; CASP8; CCDC177; CD37; CDT1; CHCHD7; CHKA; CIDEB; CNR2; | |
| | | | | | CSK; CTTN; DAGLB; DDO; DLG4; DLL3; DOC2GP; DPY19L1; EBF4; FAM134B; | |
| | | | | | FAM65B; FARSA; FLI1; FLJ31813; FOXA3; FOXK1; GARS; GLUL; GPANK1; GPSM3; | |
| | | | | | GRID1; HAL; HLA-DQA1; HOXA-AS3; | |
| | | | | | HOXA4; HOXA5; HOXC4; HSPA12B; IGFBP5; IL17C; IQCH; IZUMO1; KCNK7; KCTD11; | |
| | | | | | KDM2B; KRT18; KRT8; LAIR2; LAMA1; LAMB2; LAMC3; LCE3A; LINC00421; LMTK3; | |
| | | | | | LOC146880; LOC399829; LOC400043; LOC728392; LRCOL1; LTB4R; LTB4R2; MAP3K7CL; | |
| | | | | | MBP; MCCC1; METAP2; MFI2; MIR548N; MIR596; MRAS; MSX1; MYO1F; MYO7A; | |
| | | | | | NAV1; NBR2; NDRG1; NINJ2; NLRP3; NMT1; NPR2; NRXN2; OR5W2; P4HB; PCDHB13; | |
| | | | | | PFN3; PLAG1; PLD4; PNPLA8; POU2AF1; PRDM2; PRDM8; PTPRN2; RAB38; RAI1; | |
| | | | | | RALGDS; RBMS1; RGL3; RNF216; RNF222; RPL23A; RPL27A; RPS6; RPS8; RRP12; | |
| | | | | | SAMD11; SEMA6B; SFT2D1; SH2B2; SH3PXD2A; SH3RF3; SH3RF3- | |
| | | | | | AS1; SH3TC1; SHANK1; SHANK2; SLC1A4; SLC6A20; SLFN12; SNORA3; SNORA45; | |
| | | | | | SNORD38A; SNORD4A; SNORD52; SNRPC; SPEG; SRGN; SYMPK; SYTL1; TACC2; | |
| | | | | | TBC1D8; TBX1; TCEA2; TEX26; TEX26-AS1; THNSL2; TMEM151B; TNFAIP2; TOX; TTN- | |
| | | | | | AS1; ZMIZ1; ZMYND15; ZNF385A; ZNF890P | |
| 20% | 100.0% | 100.0% | 100.0% | 46 | ADO; AGAP2; AGAP2- | 27 |
| | | | | | AS1; BCL11B; C8orf49; CDT1; DOC2GP; EBF4; FAM65B; GARS; HOXA- | |
| | | | | | AS3; HOXA4; HOXA5; HOXC4; KCNK7; LAMA1; LAMB2; LOC728392; MFI2; MYO1F; | |
| | | | | | NAV1; NINJ2; NLRP3; RNF216; SH3RF3; SH3RF3-AS1; ZMIZ1 | |
| 25% | 100.0% | 81.8% | 99.1% | 10 | C8orf49; EBF4; FAM65B; HOXC4; KCNK7; MYO1F | 6 |

TABLE 12
Cross-validation results for different effect-size (absolute delta beta, |Δβ|) thresholds at p-value ≦0.005. Shown are the specificity (Spec)
and sensitivity (Sens) of the LOO procedure. Specificity is for 1056 normal blood samples derived from GEO (Spec GEO). The total
number of significant sites (CGs) in the resulting “Kabuki signature” set, the gene names (Names) and their total number (Genes)
corresponding to the significant sites are provided. The p-values are corrected for multiple testing (Benjamini-Hochberg correction).

| | p-value ≦0.005 | | | | | |
|---|---|---|---|---|---|---|
| | | | Spec | | | |
| \|Δβ\| | Spec | Sens | (GEO) | CGs | Names | Genes |
| 5% | 100.0% | 90.9% | 100.0% | 8490 | Not shown | 4680 |
| 10% | 100.0% | 90.9% | 100.0% | 1569 | Not shown | 902 |
| 15% | 100.0% | 100.0% | 100.0% | 267 | ADAMTS2; ADO; AGAP2; AGAP2-AS1; ANKRD26P3; | 150 |
| | | | | | ARHGAP35; ASB2; B4GALT5; BCL11B; BPI; BRCA1; C14orf182; C6orf48; C8orf49; | |
| | | | | | CASP8; CCDC177; CD37; CDT1; CHCHD7; CHKA; CIDEB; CNR2; CSK; CTTN; DAGLB; | |
| | | | | | DDO; DLG4; DLL3; DOC2GP; DPY19L1; EBF4; FAM134B; FAM65B; FARSA; FLI1; | |
| | | | | | FLJ31813; FOXA3; FOXK1; GARS; GLUL; GPANK1; GPSM3; GRID1; HAL; HOXA- | |
| | | | | | AS3; HOXA4; HOXA5; HOXC4; HSPA12B; IGFBP5; IL17C; IQCH; IZUMO1; KCNK7; | |
| | | | | | KCTD11; KDM2B; KRT18; KRT8; LAIR2; LAMA1; LAMB2; LAMC3; LCE3A; LINC00421; | |
| | | | | | LMTK3; LOC146880; LOC399829; LOC400043; LOC728392; LRCOL1; LTB4R; | |
| | | | | | LTB4R2; MAP3K7CL; MBP; MCCC1; METAP2; MFI2; MIR548N; MIR596; MRAS; | |
| | | | | | MSX1; MYO1F; MYO7A; NAV1; NBR2; NDRG1; NINJ2; NLRP3; NMT1; NPR2; NRXN2; | |
| | | | | | OR5W2; P4HB; PCDHB13; PFN3; PLAG1; PLD4; PNPLA8; POU2AF1; PRDM2; PTPRN2; | |
| | | | | | RAB38; RAI1; RALGDS; RGL3; RNF216; RNF222; RPL23A; RPL27A; RPS6; | |
| | | | | | RPS8; RRP12; SAMD11; SEMA6B; SFT2D1; SH2B2; SH3PXD2A; SH3RF3; SH3RF3- | |
| | | | | | AS1; SH3TC1; SHANK1; SHANK2; SLC1A4; SLC6A20; SLFN12; SNORA3; SNORA45; | |
| | | | | | SNORD38A; SNORD4A; SNORD52; SNRPC; SPEG; SRGN; SYMPK; SYTL1; TACC2; | |
| | | | | | TBC1D8; TBX1; TCEA2; TEX26; TEX26-AS1; THNSL2; TMEM151B; TNFAIP2; TOX; | |
| | | | | | TTN-AS1; ZMIZ1; ZMYND15; ZNF385A; ZNF890P | |
| 20% | 100.0% | 100.0% | 100.0% | 46 | ADO; AGAP2; AGAP2-AS1; BCL11B; C8orf49; CDT1; DOC2GP; EBF4; | 27 |
| | | | | | FAM65B; GARS; HOXA- AS3; HOXA4; HOXA5; HOXC4; KCNK7; LAMA1; LAMB2; | |
| | | | | | LOC728392; MFI2; MYO1F; NAV1; NINJ2; NLRP3; RNF216; SH3RF3; SH3RF3-AS1; ZMIZ1 | |
| 25% | 100.0% | 90.9% | 99.1% | 10 | C8orf49; EBF4; FAM65B; HOXC4; KCNK7; MYO1F | 6 |

TABLE 13
Cross-validation results for different effect-size (absolute delta beta, |Δβ|) thresholds at p-value ≦0.001. Shown
are the specificity (Spec) and sensitivity (Sens) of the LOO procedure. Specificity is for 1056 normal blood samples derived from
GEO (Spec GEO). The total number of significant sites (CGs) in the resulting “Kabuki signature” set, the gene names (Names)
and their total number (Genes) corresponding to the significant sites are provided. The p-values are corrected for multiple testing
(Benjamini-Hochberg correction).

| | p-value ≦0.001 | | | | | |
|---|---|---|---|---|---|---|
| | | | Spec | | | |
| \|Δβ\| | Spec | Sens | (GEO) | CGs | Names | Genes |
| 5% | 100.0% | 90.9% | 100.0% | 5492 | Not shown | 3337 |
| 10% | 100.0% | 100.0% | 100.0% | 1248 | Not shown | 745 |
| 15% | 100.0% | 100.0% | 100.0% | 232 | ADAMTS2; ADO; AGAP2; AGAP2- | 130 |
| | | | | | AS1; ANKRD26P3; ARHGAP35; ASB2; B4GALT5; BCL11B; BPI; C14orf182; | |
| | | | | | C6orf48; CASP8; CCDC177; CD37; CDT1; CHCHD7; CHKA; CIDEB; | |
| | | | | | CNR2; CSK; DAGLB; DDO; DLG4; DOC2GP; DPY19L1; EBF4; FAM134B; | |
| | | | | | FAM65B; FARSA; FLI1; FLJ31813; FOXA3; FOXK1; GARS; GLUL; | |
| | | | | | GPANK1; GPSM3; GRID1; HAL; HOXA- | |
| | | | | | AS3; HOXA4; HOXA5; HOXC4; HSPA12B; IGFBP5; IL17C; IQCH; KCNK7; | |
| | | | | | KCTD11; KDM2B; KRT18; KRT8; LAIR2; LAMA1; LAMB2; LAMC3; LCE3A; | |
| | | | | | LINC00421; LMTK3; LOC146880; LOC400043; LOC728392; LRCOL1; | |
| | | | | | LTB4R; LTB4R2; MAP3K7CL; MBP; MCCC1; METAP2; MFI2; MIR548N; | |
| | | | | | MRAS; MSX1; MYO1F; MYO7A; NAV1; NLRP3; NPR2; NRXN2; | |
| | | | | | OR5W2; P4HB; PCDHB13; PFN3; PLAG1; PLD4; PNPLA8; POU2AF1; | |
| | | | | | PRDM2; RGL3; RNF216; RNF222; RPL23A; RPL27A; RPS6; RPS8; RRP12; | |
| | | | | | SAMD11; SEMA6B; SFT2D1; SH2B2; SH3PXD2A; SH3RF3; SH3RF3- | |
| | | | | | AS1; SH3TC1; SHANK1; SLC1A4; SLC6A20; SNORA3; SNORA45; SNORD38A; | |
| | | | | | SNORD4A; SNORD52; SNRPC; SPEG; SRGN; SYMPK; SYTL1; | |
| | | | | | TACC2; TBC1D8; TCEA2; THNSL2; TMEM151B; TNFAIP2; TOX; TTN-AS1; | |
| | | | | | ZMIZ1; ZMYND15; ZNF385A; ZNF890P | |
| 20% | 100.0% | 100.0% | 100.0% | 43 | ADO; AGAP2; AGAP2- | 24 |
| | | | | | AS1; BCL11B; CDT1; EBF4; FAM65B; GARS; HOXA- | |
| | | | | | AS3; HOXA4; HOXA5; HOXC4; KCNK7; LAMA1; LAMB2; LOC728392; | |
| | | | | | MFI2; MYO1F; NAV1; NLRP3; RNF216; SH3RF3; SH3RF3-AS1; ZMIZ1 | |
| 25% | 100.0% | 90.9% | 98.9% | 9 | EBF4; FAM65B; HOXC4; KCNK7; MYO1F | 5 |

TABLE 14
Cross-validation results for different effect-size (absolute delta beta, |Δβ|) thresholds at p-value ≦0.0001. Shown
are the specificity (Spec) and sensitivity (Sens) of the LOO procedure. Specificity is for 1056 normal blood samples derived from
GEO (Spec GEO). The total number of significant sites (CGs) in the resulting “Kabuki signature” set, the gene names (Names) and
their total number (Genes) corresponding to the significant sites are provided. The p-values are corrected for multiple testing
(Benjamini-Hochberg correction).

| | p-value ≦0.0001 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 100.0% | 2696 | Not shown | 1822 |
| 10% | 100.0% | 100.0% | 100.0% | 801 | Not shown | 504 |
| 15% | 100.0% | 100.0% | 100.0% | 181 | ADO; AGAP2; AGAP2- | 104 |
| | | | | | AS1; ARHGAP35; ASB2; BCL11B; BPI; C14orf182; C6orf48; CASP8; CCDC177; CD37; CDT1; | |
| | | | | | CHCHD7; CHKA; CIDEB; CNR2; CSK; DAGLB; EBF4; FAM134B; FAM65B; FLJ31813; | |
| | | | | | FOXK1; GARS; GPANK1; GPSM3; GRID1; HAL; HOXA- | |
| | | | | | AS3; HOXA4; HOXA5; HOXC4; HSPA12B; IGFBP5; IL17C; IQCH; KCNK7; KCTD11; | |
| | | | | | KDM2B; KRT18; KRT8; LAIR2; LAMA1; LAMB2; LCE3A; LMTK3; LOC146880; LOC400043; | |
| | | | | | LRCOL1; LTB4R; LTB4R2; METAP2; MFI2; MIR548N; MSX1; MYO1F; MYO7A; NAV1; | |
| | | | | | NLRP3; NRXN2; P4HB; PCDHB13; PFN3; PLAG1; PLD4; PNPLA8; PRDM2; RGL3; | |
| | | | | | RNF216; RPL23A; RPL27A; RPS6; RPS8; RRP12; SAMD11; SEMA6B; SH2B2; SH3PXD2A; | |
| | | | | | SH3RF3; SH3RF3- | |
| | | | | | AS1; SH3TC1; SHANK1; SLC1A4; SLC6A20; SNORA3; SNORA45; SNORD38A; SNORD4A; | |
| | | | | | SNORD52; SPEG; SRGN; SYTL1; TACC2; TBC1D8; TCEA2; THNSL2; TMEM151B; | |
| | | | | | TNFAIP2; TOX; TTN-AS1; ZMIZ1; ZMYND15; ZNF385A | |
| 20% | 100.0% | 100.0% | 100.0% | 39 | ADO; AGAP2; AGAP2-AS1; BCL11B; CDT1; EBF4; FAM65B; GARS; HOXA- | 23 |
| | | | | | AS3; HOXA4; HOXA5; HOXC4; KCNK7; LAMA1; LAMB2; MFI2; MYO1F; NAV1; NLRP3; | |
| | | | | | RNF216; SH3RF3; SH3RF3-AS1; ZMIZ1 | |
| 25% | 100.0% | 81.8% | 98.9% | 9 | EBF4; FAM65B; HOXC4; KCNK7; MYO1F | 5 |

TABLE 15
Cross-validation results for different effect-size (absolute delta beta, |Δβ|) thresholds at p-value ≦0.00001. Shown are the specificity (Spec) and sensitivity (Sens) of the LOO
procedure. Specificity is for 1056 normal blood samples derived from GEO (Spec GEO). The total number of significant sites (CGs) in the resulting “Kabuki signature” set, the gene names (Names)
and their total number (Genes) corresponding to the significant sites are provided. The p-values are corrected for multiple testing (Benjamini-Hochberg correction).

| | p-value ≦0.00001 | | | | | |
|---|---|---|---|---|---|---|
| | | | Spec | | | |
| \|Δβ\| | Spec | Sens | (GEO) | CGs | Names | Genes |
| 5% | 100.0% | 100.0% | 100.0% | 1188 | Not shown | 893 |
| 10% | 100.0% | 100% | 100% | 447 | ADO; AFAP1; AFAP1-AS1; AGAP2; AGAP2- | 314 |
| | | | | | AS1; AKT3; ANKRD30B; ANXA6; ARHGAP31; ARHGAP32; ARHGEF7; ARL5C; ARPC1B; ARSG; ASB2; ASUN; ATP11A; ATP6V1G2- | |
| | | | | | DDX39B; AXIN1; BAG2; BAHCC1; BCL11B; BRD2; C10orf11; C12orf79; C1orf53; C6orf48; C6orf62; C9orf106; CACNA1H; CACNG8; CAMTA1; | |
| | | | | | CAPZB; CASP8; CASZ1; CBLN2; CCDC88A; CCT7; CD37; CDT1; CIAPIN1; CNR2; CNST; CNTN5; COMT; COQ2; COQ9; COX4I2; CRIP2; | |
| | | | | | CSK; CSNK2B; CSRP2BP; CXXC1; DAGLB; DBX2; DDX39A; DDX39B; DGKI; DIO2; DIO2- | |
| | | | | | AS1; DLG4; DOK1; DZIP3; EBF4; EEF1D; EFNA1; EMILIN2; ERH; ETS1; EVA1B; EVC2; EXOC8; FAM110D; FAM63A; FAM65B; FGF20; FIGNL2; | |
| | | | | | FLCN; FLJ12825; FLJ31813; FNDC3B; FOXK1; FOXN3; FST; FYB; GARS; GAS5; GMDS- | |
| | | | | | AS1; GNG4; GNG7; GOLGA3; GPANK1; GPSM3; GUK1; HAL; HBP1; HDAC4; HIC1; HLA- | |
| | | | | | DOA; HLX; HNRNPA1; HNRNPA1P10; HNRNPH1; HOXA- | |
| | | | | | AS3; HOXA4; HOXA5; HOXA6; HOXC4; HSPA12B; IBTK; IGFBP5; IL17C; IL17RE; INPP5E; IRAK3; JMJD1C; KAT6A; KCNA2; KCNAB1; KCNK7; | |
| | | | | | KDM2B; KDM3A; KIAA1524; KIRREL3; KLHDC7B; KRT18; KRT8; KTI12; LAMA1; LAMB2; LAMP1; LHX6; LMTK3; LOC146880; LOC389705 | |
| | | | | | LONP2; LOXL3; LPAR5; LPAR6; LRBA; LSM3; LSR; LTB4R; LTBR; LYAR; MAB21L2; MAP3K6; MARS2; MBNL1; MBOAT2; MDH1; MEN1; | |
| | | | | | METAP2; MFI2; MICAL3; MIR1296; MIR4285; MIR4520B; MIR4763; MIR548AE2; MIR548N; MIRLET7A3; MIRLET7B; MIRLET7BHG; MRPL44; | |
| | | | | | MRPS15; MRPS18B; MSX1; MXRA8; MYL1; MYO1F; NAV1; NDUFA3; NFXL1; NPM1; NRXN2; NTMT1; NUFIP2; OR5B17; ORAI2; OSCAR; | |
| | | | | | P4HB; PAPPA; PARK7; PARP4; PCF11; PCGF3; PDE4A; PDE6A; PET117; PEX13; PFN3; PHGDH; PKP1; PLD4; PLIN5; PM20D2; PNO1; POLE2; | |
| | | | | | PPP1R10; PRDM2; PSMD14; PTDSS2; PTPN6; PURG; PUS10; RAB11FIP3; RASAL1; RASGRP2; RB1; RBFOX3; RMDN2; RMDN2- | |
| | | | | | AS1; RNF216; RNF38; RPAP3; RPL14; RPL23A; RPL35; RPL37; RPS12; RPS18; RPS6; RPS8; RPSA; RRP12; RUFY1; SAMD11; SCARF2; SCNN1A; | |
| | | | | | SEC31B; SEMA6B; SERTAD4; SH2B2; SH3PXD2A; SH3RF3; SH3RF3- | |
| | | | | | AS1; SH3TC1; SIX2; SLC16A6; SLC17A5; SLC1A4; SLC33A1; SLC39A9; SLC50A1; SLC6A20; SLCO3A1; SLMAP; SMIM8; SMYD2; SNORA33; | |
| | | | | | SNORA6; SNORD100; SNORD38A; SNORD44; SNORD4A; SNORD52; SNORD72; SNORD75; SNORD76; SNORD77; SNORD78; SNORD79; | |
| | | | | | SNORD80; SNX27; SNX6; SPRTN; SRGN; SRSF1; SSTR5-AS1; STAU2- | |
| | | | | | AS1; TACC2; TAF8; TAOK3; TAP2; TBC1D22A; TBC1D8; TBX4; TCERG1L; TCFL5; TCHH; TFAP2E; TFB2M; TGFBI; THNSL2; TNFAIP2; TNR; | |
| | | | | | TOX; TRAK1; TRIM67; TTN-AS1; TVP23A; TXNDC12; UBA6; UBA6- | |
| | | | | | AS1; UBE2J2; UBE2R2; UPF1; USP42; VOPP1; VPS52; WDR37; WRN; YAP1; ZBTB49; ZC3H12D; ZFAND2A; ZKSCAN1; ZMIZ1; ZMYND15; | |
| | | | | | ZNF385A; ZNF787; ZSWIM8; ZSWIM8-AS1 | |
| 15% | 100.0% | 100% | 100% | 111 | ADO; AGAP2; AGAP2- | 67 |
| | | | | | AS1; ASB2; BCL11B; C6orf48; CASP8; CD37; CDT1; CNR2; CSK; DAGLB; EBF4; FAM65B; FLJ31813; FOXK1; GARS; GPANK1; GPSM3; HAL; | |
| | | | | | HOXA- | |
| | | | | | AS3; HOXA4; HOXA5; HOXC4; HSPA12B; IGFBP5; KDM2B; KRT18; KRT8; LAMA1; LAMB2; LOC146880; LTB4R; METAP2; MFI2; MIR548N; | |
| | | | | | MYO1F; NAV1; NRXN2; PFN3; PLD4; PRDM2; RNF216; RPL23A; RPS6; RPS8; RRP12; SAMD11; SEMA6B; SH3PXD2A; SH3RF3; SH3RF3- | |
| | | | | | AS1; SH3TC1; SLC1A4; SLC6A20; SNORD38A; SNORD4A; SNORD52; SRGN; TACC2; TBC1D8; THNSL2; TNFAIP2; TOX; TTN- | |
| | | | | | AS1; ZMIZ1; ZMYND15 | |
| 20% | 100.0% | 100% | 100% | 29 | ADO; AGAP2; AGAP2- | 18 |
| | | | | | AS1; BCL11B; CDT1; EBF4; FAM65B; GARS; HOXA4; HOXC4; LAMA1; LAMB2; MFI2; MYO1F; RNF216; SH3RF3; SH3RF3-AS1; ZMIZ1 | |
| 25% | 100.0% | 90.9% | 99.7% | 6 | FAM65B; HOXC4; MYO1F | 3 |

TABLE 16
Three additional CpG loci corresponding to two genes were identified as showing a statistically significant (corrected p
value <0.01) difference in CS and non-CS controls. “Mean not-CHARGE refers to the mean β-value for the CpG loci in the non-CS
cases “Mean CHARGE” refers to the mean β-value for the CpG loci in the CS samples.

| | | Benjamini- | | | | | | | |
|---|---|---|---|---|---|---|---|---|---|
| | | Hochberg | | | DNA | | | | |
| | | corrected p- | | Absolute | methylation | Mean not- | Mean | Gene | |
| Illumina ID | p-value | value | deltaBeta | deltaBeta | effect | CHARGE | CHARGE | Symbol | Genome_Build |
| cg14422498 | 1.33E−06 | 0.00308636 | −0.10816215 | 0.10816215 | LOSS | 0.3808205 | 0.272658 | | 37 |
| cg18657389 | 3.17E−06 | 0.00541815 | −0.10816215 | 0.11427340 | LOSS | 0.7313574 | 0.617084 | EVPL | 37 |
| cg25285743 | 7.66E−07 | 0.00228633 | 0.10211681 | 0.10211681 | GAIN | 0.5901659 | 0.692283 | LMO3 | 37 |

| | | | Genomic | | |
|---|---|---|---|---|---|
| | | | Coordinate | | Relation to |
| | | | (NCBI, | | transcription |
| Illumina ID | Strand | Chromosome | hg19) | Relation_to_UCSC_CpG_Island | start site (TSS) |
| cg14422498 | R | 9 | 100639423 | | |
| cg18657389 | R | 17 | 74023630 | | EVPL (tss200) |
| cg25285743 | F | 12 | 16701533 | | LMO3 |
| | | | | | (3′UTR); |
| | | | | | LMO3 (3′UTR) |

TABLE 17
75 additional CpG loci corresponding to 28 Genes were identified as showing a statistically significant (p-value ≦0.05) difference in KS and
non-KS controls. “Mean not-Kabuki” refers to the mean beta-value for the CpG loci in the non-KS cases. “Mean Kabuki” refers to the mean beta-value
for the CpG loci in the KS samples.

| | | Benjamini- | | | | | | |
|---|---|---|---|---|---|---|---|---|
| | | Hochberg | | | DNA | | | |
| | | corrected p- | | absDelta | Methylation | Mean not- | | |
| Illumina ID | p-value | value | deltaBeta | Beta | Effect | Kabuki | Mean Kabuki | Gene Symbol |
| cg03657281 | 0.000545826 | 0.039123657 | −0.22287617 | 0.222876166 | Loss | 0.707974691 | 0.485098525 | SLC22A23; SLC22A23; |
| | | | | | | | | SLC22A23; SLC22A23 |
| cg24169822 | 2.96E−06 | 0.003253178 | −0.20777829 | 0.207778288 | Loss | 0.447022355 | 0.239244067 | HOXA4 |
| cg03724423 | 1.78E−05 | 0.007879037 | −0.18951298 | 0.189512977 | Loss | 0.381331218 | 0.191818242 | HOXA4 |
| cg04321618 | 2.96E−06 | 0.003253178 | −0.1859374 | 0.1859374 | Loss | 0.4195601 | 0.2336227 | HOXA4 |
| cg14700524 | 9.91E−05 | 0.017451795 | −0.17890157 | 0.178901568 | Loss | 0.815736418 | 0.63683485 | |
| cg12876594 | 4.44E−05 | 0.012044763 | −0.17715184 | 0.177151839 | Loss | 0.497182273 | 0.320030433 | NPR2 |
| cg04317399 | 9.91E−05 | 0.017451795 | −0.17562929 | 0.175629286 | Loss | 0.390439536 | 0.21481025 | HOXA4; HOXA4 |
| cg24869272 | 6.66E−05 | 0.014525204 | −0.17496219 | 0.174962191 | Loss | 0.409302991 | 0.2343408 | TSPAN4; TSPAN4; |
| | | | | | | | | TSPAN4; TSPAN4; |
| | | | | | | | | TSPAN4; TSPAN4; |
| | | | | | | | | TSPAN4 |
| cg05783384 | 1.04E−05 | 0.006001284 | −0.17302223 | 0.17302223 | Loss | 0.789634755 | 0.616612525 | |
| cg06942814 | 2.81E−05 | 0.009696319 | −0.17288902 | 0.17288902 | Loss | 0.515048645 | 0.342159625 | HOXA4 |
| cg07967717 | 1.48E−06 | 0.002458452 | −0.17177699 | 0.171776986 | Loss | 0.368327636 | 0.19655065 | CNR2 |
| cg17457637 | 0.000205609 | 0.024756111 | −0.16984355 | 0.169843545 | Loss | 0.386699145 | 0.2168556 | HOXA4 |
| cg25952581 | 1.48E−06 | 0.002458452 | −0.16970611 | 0.169706108 | Loss | 0.4250646 | 0.255358492 | HOXA4 |
| cg23510089 | 2.81E−05 | 0.009696319 | −0.16888611 | 0.168886111 | Loss | 0.767464827 | 0.598578717 | |
| cg07548255 | 9.91E−05 | 0.017451795 | −0.16867061 | 0.168670608 | Loss | 0.585514391 | 0.416843783 | SH3RF3; SH3RF3- |
| | | | | | | | | AS1; SH3RF3-AS1 |
| cg13935577 | 4.44E−05 | 0.012044763 | −0.16856998 | 0.168569985 | Loss | 0.783746818 | 0.615176833 | BTBD11; BTBD11 |
| cg24201793 | 1.48E−06 | 0.002458452 | −0.16739174 | 0.167391739 | Loss | 0.648610864 | 0.481219125 | MBOAT2 |
| cg25702651 | 6.66E−05 | 0.014525204 | −0.16688422 | 0.166884217 | Loss | 0.4287815 | 0.261897283 | |
| cg02483029 | 0.000545826 | 0.039123657 | −0.16379654 | 0.163796542 | Loss | 0.701013409 | 0.537216867 | SPIDR; SPIDR; SPIDR; |
| | | | | | | | | SPIDR |
| cg23884241 | 2.96E−06 | 0.003253178 | −0.16298323 | 0.162983233 | Loss | 0.5839747 | 0.420991467 | HOXA4 |
| cg11685316 | 0.000736644 | 0.045047655 | −0.16281272 | 0.162812723 | Loss | 0.737778573 | 0.57496585 | MFSD6L; MFSD6L |
| cg19497523 | 9.91E−05 | 0.017451795 | −0.16142362 | 0.16142362 | Loss | 0.850669945 | 0.689246325 | TMPRSS9 |
| cg08883485 | 0.000205609 | 0.024756111 | −0.16112403 | 0.16112403 | Loss | 0.484720964 | 0.323596933 | NAV1 |
| cg15122841 | 5.92E−06 | 0.004562719 | −0.15940461 | 0.159404613 | Loss | 0.807004455 | 0.647599842 | HDAC4 |
| cg21801165 | 1.48E−06 | 0.002458452 | −0.15885777 | 0.158857765 | Loss | 0.530243582 | 0.371385817 | |
| cg15630950 | 9.91E−05 | 0.017451795 | −0.15795437 | 0.157954371 | Loss | 0.604374155 | 0.446419783 | HLA-DOA |
| cg03534375 | 2.96E−06 | 0.003253178 | −0.15335462 | 0.153354621 | Loss | 0.535936055 | 0.382581433 | SLMAP |
| cg00921309 | 1.78E−05 | 0.007879037 | −0.15220007 | 0.152200067 | Loss | 0.5088694 | 0.356669333 | |
| cg02713669 | 6.66E−05 | 0.014525204 | −0.15216219 | 0.152162194 | Loss | 0.292733764 | 0.14057157 | SH3RF3; SH3RF3- |
| | | | | | | | | AS1; SH3RF3-AS1 |
| cg26040809 | 0.000545826 | 0.039123657 | −0.1514797 | 0.151479697 | Loss | 0.829734064 | 0.678254367 | ADARB2 |
| cg07021906 | 5.92E−06 | 0.004562719 | −0.15122097 | 0.15122097 | Loss | 0.736244036 | 0.585023067 | SLC7A5 |
| cg02142461 | 1.48E−06 | 0.002458452 | −0.15102594 | 0.151025942 | Loss | 0.568091 | 0.417065058 | LYAR; LYAR; ZBTB49 |
| cg00562553 | 2.96E−06 | 0.003253178 | −0.15088227 | 0.15088227 | Loss | 0.801209545 | 0.650327275 | HOXA4 |
| cg26125366 | 1.48E−06 | 0.002458452 | −0.1508059 | 0.150805895 | Loss | 0.361926945 | 0.21112105 | |
| cg14270725 | 5.92E−06 | 0.004562719 | −0.15076771 | 0.150767705 | Loss | 0.317108145 | 0.16634044 | MXRA8; MXRA8; |
| | | | | | | | | MXRA8; MXRA8; |
| | | | | | | | | MXRA8 |
| cg23926439 | 0.000545826 | 0.039123657 | 0.150255174 | 0.150255174 | Gain | 0.535782309 | 0.68603748 | |
| cg02661079 | 2.81E−05 | 0.009696319 | 0.151650952 | 0.151650952 | Gain | 0.364989373 | 0.516640325 | CDH22 |
| cg10193721 | 9.91E−05 | 0.017451795 | 0.152593035 | 0.152593035 | Gain | 0.190991682 | 0.343584717 | LTB4R; LTB4R2; CIDEB; |
| | | | | | | | | CIDEB; LTB4R2 |
| cg12301347 | 4.44E−05 | 0.012044763 | 0.152873337 | 0.152873337 | Gain | 0.288168655 | 0.441041992 | |
| cg08352439 | 1.48E−06 | 0.002458452 | 0.153206312 | 0.153206312 | Gain | 0.582253655 | 0.735459967 | VOPP1 |
| cg24363820 | 0.000205609 | 0.024756111 | 0.153494638 | 0.153494638 | Gain | 0.476936145 | 0.630430783 | CPT1B; CPT1B; CPT1B; |
| | | | | | | | | CPT1B; CPT1B; |
| | | | | | | | | CPT1B; CPT1B; |
| | | | | | | | | CPT1B; CPT1B; CPT1B; |
| | | | | | | | | CHKB-CPT1B |
| cg27619353 | 1.04E−05 | 0.006001284 | 0.154038076 | 0.154038076 | Gain | 0.388803791 | 0.542841867 | LGALS1 |
| cg25334934 | 5.92E−06 | 0.004562719 | 0.15465902 | 0.15465902 | Gain | 0.651384455 | 0.806043475 | |
| cg10290504 | 0.000205609 | 0.024756111 | 0.154996448 | 0.154996448 | Gain | 0.199256269 | 0.354252717 | |
| cg10770023 | 0.000205609 | 0.024756111 | 0.155278024 | 0.155278024 | Gain | 0.452634409 | 0.607912433 | CPT1B; CPT1B; CPT1B; |
| | | | | | | | | CPT1B; CPT1B; |
| | | | | | | | | CPT1B; CPT1B; |
| | | | | | | | | CPT1B; CHKB- |
| | | | | | | | | CPT1B |
| cg26631039 | 0.000143483 | 0.020691318 | 0.155448474 | 0.155448474 | Gain | 0.137210776 | 0.29265925 | GLI2 |
| cg05654765 | 0.000143483 | 0.020691318 | 0.157465431 | 0.157465431 | Gain | 0.389924327 | 0.547389758 | LAMB2; LAMB2 |
| cg08498747 | 1.48E−06 | 0.002458452 | 0.158076749 | 0.158076749 | Gain | 0.569288509 | 0.727365258 | |
| cg16276982 | 0.000736644 | 0.045047655 | 0.158794724 | 0.158794724 | Gain | 0.306828109 | 0.465622833 | |
| cg26986681 | 0.000400864 | 0.033829502 | 0.160018319 | 0.160018319 | Gain | 0.231060573 | 0.391078892 | IGFBP7-AS1 |
| cg16081457 | 2.96E−06 | 0.003253178 | 0.1612716 | 0.1612716 | Gain | 0.5209443 | 0.6822159 | |
| cg05156901 | 0.000736644 | 0.045047655 | 0.161506804 | 0.161506804 | Gain | 0.555427955 | 0.716934758 | CPT1B; CPT1B; CPT1B; |
| | | | | | | | | CPT1B; CPT1B; |
| | | | | | | | | CPT1B; CPT1B; |
| | | | | | | | | CPT1B; CHKB- |
| | | | | | | | | CPT1B |
| cg22344745 | 0.000143483 | 0.020691318 | 0.162023911 | 0.162023911 | Gain | 0.537765064 | 0.699788975 | |
| cg20517050 | 1.78E−05 | 0.007879037 | 0.162136438 | 0.162136438 | Gain | 0.650460645 | 0.812597083 | HOXA5; HOXA- |
| | | | | | | | | AS3 |
| cg01308968 | 0.000545826 | 0.039123657 | 0.164175747 | 0.164175747 | Gain | 0.575034036 | 0.739209783 | IGFBP7-AS1 |
| cg12765123 | 0.000400864 | 0.033829502 | 0.165860469 | 0.165860469 | Gain | 0.574899173 | 0.740759642 | |
| cg03294458 | 0.000545826 | 0.039123657 | 0.166611545 | 0.166611545 | Gain | 0.12960339 | 0.296214935 | WNK4 |
| cg10932486 | 1.04E−05 | 0.006001284 | 0.167217033 | 0.167217033 | Gain | 0.556037109 | 0.723254142 | |
| cg02916332 | 1.78E−05 | 0.007879037 | 0.168286167 | 0.168286167 | Gain | 0.608908 | 0.777194167 | HOXA5; HOXA- |
| | | | | | | | | AS3 |
| cg26310551 | 0.000288445 | 0.028858761 | 0.168287927 | 0.168287927 | Gain | 0.252093282 | 0.420381208 | LTB4R; LTB4R2; CIDEB; |
| | | | | | | | | CIDEB; LTB4R2 |
| cg17432857 | 1.04E−05 | 0.006001284 | 0.168403377 | 0.168403377 | Gain | 0.606293673 | 0.77469705 | HOXA5; HOXA- |
| | | | | | | | | AS3 |
| cg07330481 | 1.48E−06 | 0.002458452 | 0.169538806 | 0.169538806 | Gain | 0.573050227 | 0.742589033 | ARL5C; ARL5C |
| cg23129930 | 6.66E−05 | 0.014525204 | 0.170164367 | 0.170164367 | Gain | 0.569918991 | 0.740083358 | HOXA6; HOXA- |
| | | | | | | | | AS3; HOXA-AS3 |
| cg02248486 | 5.92E−06 | 0.004562719 | 0.174414246 | 0.174414246 | Gain | 0.672434445 | 0.846848692 | HOXA5; HOXA5; HOXA- |
| | | | | | | | | AS3 |
| cg18737081 | 2.96E−06 | 0.003253178 | 0.175031395 | 0.175031395 | Gain | 0.734303555 | 0.90933495 | ZMIZ1 |
| cg11178337 | 2.96E−06 | 0.003253178 | 0.175563736 | 0.175563736 | Gain | 0.129339864 | 0.3049036 | |
| cg11724970 | 1.04E−05 | 0.006001284 | 0.176344059 | 0.176344059 | Gain | 0.683343091 | 0.85968715 | HOXA5; HOXA- |
| | | | | | | | | AS3 |
| cg19112186 | 0.000205609 | 0.024756111 | 0.178717061 | 0.178717061 | Gain | 0.529355164 | 0.708072225 | CPT1B; CPT1B; CPT1B; |
| | | | | | | | | CPT1B; CPT1B; |
| | | | | | | | | CPT1B; CPT1B; |
| | | | | | | | | CPT1B; CHKB- |
| | | | | | | | | CPT1B |
| cg27053299 | 1.48E−06 | 0.002458452 | 0.18052838 | 0.18052838 | Gain | 0.557516736 | 0.738045117 | CLYBL |
| cg02721176 | 0.000400864 | 0.033829502 | 0.186840873 | 0.186840873 | Gain | 0.256788927 | 0.4436298 | CCDC172 |
| cg02005600 | 5.92E−06 | 0.004562719 | 0.190667314 | 0.190667314 | Gain | 0.654958836 | 0.84562615 | HOXA5; HOXA- |
| | | | | | | | | AS3 |
| cg26516362 | 0.000400864 | 0.033829502 | 0.193294684 | 0.193294684 | Gain | 0.271304049 | 0.464598733 | RUFY1; RUFY1; RUFY1 |
| cg19759481 | 1.04E−05 | 0.006001284 | 0.216017614 | 0.216017614 | Gain | 0.605454836 | 0.82147245 | HOXA5; HOXA5; HOXA- |
| | | | | | | | | AS3 |
| cg20354552 | 0.000736644 | 0.045047655 | 0.220332162 | 0.220332162 | Gain | 0.08997291 | 0.310305072 | SLFN12 |
| cg20744163 | 2.96E−06 | 0.003253178 | 0.23788097 | 0.23788097 | Gain | 0.723305764 | 0.961186733 | ZMIZ1 |

| | | | Genomic | | | Relation to |
|---|---|---|---|---|---|---|
| | | | Coordinate | | | transcription start |
| Illumina ID | Genome_Build | Chromosome | (NCBI, hg19) | Strand | Relation_to_UCSC_CpG_Island | site (TSS) |
| cg03657281 | 37 | 6 | 3270030 | F | | SLC22A23 (body) |
| cg24169822 | 37 | 7 | 27170994 | F | S_Shore | HOXA4 (tss1500) |
| cg03724423 | 37 | 7 | 27170755 | R | S_Shore | HOXA4 (tss1500) |
| cg04321618 | 37 | 7 | 27170880 | R | S_Shore | HOXA4 (tss1500) |
| cg14700524 | 37 | 10 | 3282231 | R | | |
| cg12876594 | 37 | 9 | 35791798 | F | Island | NPR2 (tss1500) |
| cg04317399 | 37 | 7 | 27170313 | F | Island | HOXA4 (body) |
| cg24869272 | 37 | 11 | 850296 | R | Island | TSPAN4 (body) |
| cg05783384 | 37 | 2 | 218843735 | R | Island | |
| cg06942814 | 37 | 7 | 27170819 | F | S_Shore | HOXA4 (tss1500) |
| cg07967717 | 37 | 1 | 24229682 | F | S_Shore | CNR2 (body) |
| cg17457637 | 37 | 7 | 27170717 | F | S_Shore | HOXA4 (tss1500) |
| cg25952581 | 37 | 7 | 27170961 | R | S_Shore | HOXA4 (tss1500) |
| cg23510089 | 37 | 4 | 73531188 | F | | |
| cg07548255 | 37 | 2 | 109746754 | F | Island | SH3RF3 (body); SH3RF |
| | | | | | | 3-AS1 (tss200) |
| cg13935577 | 37 | 12 | 107974897 | R | Island | BTBD11 (body) |
| cg24201793 | 37 | 2 | 9144764 | F | S_Shore | MBOAT2 (tss1500) |
| cg25702651 | 37 | 3 | 192675515 | R | | |
| cg02483029 | 37 | 8 | 48297271 | R | | SPIDR (body) |
| cg23884241 | 37 | 7 | 27169957 | R | Island | HOXA4 (body) |
| cg11685316 | 37 | 17 | 8702564 | R | Island | MFSD6L (body) |
| cg19497523 | 37 | 19 | 2425476 | R | Island | TMPRSS9 (body) |
| cg08883485 | 37 | 1 | 201619787 | F | Island | NAV1 (body) |
| cg15122841 | 37 | 2 | 240181892 | F | | HDAC4 (body) |
| cg21801165 | 37 | 13 | 50210231 | F | | |
| cg15630950 | 37 | 6 | 32976897 | R | S_Shore | HLA-DOA (body) |
| cg03534375 | 37 | 3 | 57743163 | R | S_Shore | SLMAP (tss200) |
| cg00921309 | 37 | 8 | 48091487 | F | | |
| cg02713669 | 37 | 2 | 109746691 | R | Island | SH3RF3 (body); SH3RF3- |
| | | | | | | AS1 (tss200) |
| cg26040809 | 37 | 10 | 1505626 | F | N_Shore | ADARB2 (body) |
| cg07021906 | 37 | 16 | 87866833 | R | | SLC7A5 (body) |
| cg02142461 | 37 | 4 | 4293079 | R | S_Shore | LYAR (tss1500); ZBTB49 |
| | | | | | | (body) |
| cg00562553 | 37 | 7 | 27169740 | F | Island | HOXA4 (body) |
| cg26125366 | 37 | 18 | 31806577 | R | S_Shore | |
| cg14270725 | 37 | 1 | 1289806 | R | Island | MXRA8 (body) |
| cg23926439 | 37 | 1 | 228890884 | R | Island | |
| cg02661079 | 37 | 20 | 44829722 | R | Island | CDH22 (body) |
| cg10193721 | 37 | 14 | 24780691 | F | Island | CIDEB (tss200); LTB4R |
| | | | | | | (tss200); LTB4R2 (body) |
| cg12301347 | 37 | 22 | 46285638 | R | Island | |
| cg08352439 | 37 | 7 | 55637123 | F | N_Shelf | VOPP1 (body) |
| cg24363820 | 37 | 22 | 51016703 | R | Island | CHKB- |
| | | | | | | CPT1B (body); CPT1B |
| | | | | | | (body); CPT1B (tss200) |
| cg27619353 | 37 | 22 | 38071651 | F | N_Shore | LGALS1 (body) |
| cg25334934 | 37 | 2 | 121269348 | R | | |
| cg10290504 | 37 | 11 | 116578271 | F | Island | |
| cg10770023 | 37 | 22 | 51016644 | R | Island | CHKB- |
| | | | | | | CPT1B (body); CPT1B |
| | | | | | | (body); CPT1B (tss200) |
| cg26631039 | 37 | 2 | 121625022 | F | Island | GLI2 (body) |
| cg05654765 | 37 | 3 | 49170727 | F | | LAMB2 (tss200) |
| cg08498747 | 37 | 17 | 41747669 | R | | |
| cg16276982 | 37 | 15 | 29968032 | R | S_Shore | |
| cg26986681 | 37 | 4 | 58060609 | R | N_Shore | IGFBP7-AS1 (body) |
| cg16081457 | 37 | 12 | 81103680 | R | S_Shore | |
| cg05156901 | 37 | 22 | 51016646 | R | Island | CHKB- |
| | | | | | | CPT1B (body); CPT1B |
| | | | | | | (body); CPT1B (tss200) |
| cg22344745 | 37 | 1 | 227746294 | F | Island | |
| cg20517050 | 37 | 7 | 27183806 | R | Island | HOXA- |
| | | | | | | AS3 (body); HOXA5 (tss |
| | | | | | | 1500) |
| cg01308968 | 37 | 4 | 58061859 | R | Island | IGFBP7-AS1 (body) |
| cg12765123 | 37 | 10 | 132100019 | F | | |
| cg03294458 | 37 | 17 | 40935998 | R | Island | WNK4 (body) |
| cg10932486 | 37 | 5 | 61028265 | F | | |
| cg02916332 | 37 | 7 | 27183591 | F | Island | HOXA- |
| | | | | | | AS3 (body); HOXA5 (tss |
| | | | | | | 1500) |
| cg26310551 | 37 | 14 | 24780540 | F | Island | CIDEB (body); LTB4R (tss |
| | | | | | | 200); LTB4R2 (body) |
| cg17432857 | 37 | 7 | 27184438 | R | Island | HOXA- |
| | | | | | | AS3 (body); HOXA5 (tss |
| | | | | | | 1500) |
| cg07330481 | 37 | 17 | 37322330 | F | S_Shore | ARL5C (body) |
| cg23129930 | 37 | 7 | 27186993 | F | Island | HOXA- |
| | | | | | | AS3 (body); HOXA6 (body) |
| cg02248486 | 37 | 7 | 27183196 | R | Island | HOXA- |
| | | | | | | AS3 (body); HOXA5 (body) |
| cg18737081 | 37 | 10 | 80999807 | F | N_Shelf | ZMIZ1 (body) |
| cg11178337 | 37 | 17 | 43065745 | R | | |
| cg11724970 | 37 | 7 | 27182493 | R | N_Shore | HOXA- |
| | | | | | | AS3 (body); HOXA5 (body) |
| cg19112186 | 37 | 22 | 51016638 | R | Island | CHKB- |
| | | | | | | CPT1B (body); CPT1B |
| | | | | | | (body); CPT1B (tss200) |
| cg27053299 | 37 | 13 | 100548780 | F | Island | CLYBL (body) |
| cg02721176 | 37 | 10 | 118084587 | R | | CCDC172 (body) |
| cg02005600 | 37 | 7 | 27183686 | R | Island | HOXA- |
| | | | | | | AS3 (body); HOXA5 (tss |
| | | | | | | 1500) |
| cg26516362 | 37 | 5 | 178986906 | F | Island | RUFY1 (body) |
| cg19759481 | 37 | 7 | 27183401 | R | Island | HOXA- |
| | | | | | | AS3 (body); HOXA5 (tss |
| | | | | | | 200) |
| cg20354552 | 37 | 17 | 33760249 | F | | SLEN12 (tss1500) |
| cg20744163 | 37 | 10 | 80999841 | F | N_Shelf | ZMIZ1 (body) |

### REFERENCES

1. Berger, S. L., Kouzarides, T., Shiekhattar, R. & Shilatifard, A. An operational definition of epigenetics. Genes Dev 23, 781-3 (2009).
2. Turinsky, A. L. et al. DAnCER: disease-annotated chromatin epigenetics resource. Nucleic Acids Res 39, D889-94 (2010).
3. Ng, S. B. et al. Exome sequencing identifies MLL2 mutations as a cause of Kabuki syndrome. Nat Genet 42, 790-3 (2010).
4. Lederer, D. et al. Deletion of KDM6A, a Histone Demethylase Interacting with MLL2, in Three Patients with Kabuki Syndrome. Am J Hum Genet 90, 119-24 (2012).
5. Hoischen, A. et al. De novo mutations of SETBP1 cause Schinzel-Giedion syndrome. Nat Genet 42, 483-5 (2010).
6. Hoischen, A. et al. De novo nonsense mutations in ASXL1 cause Bohring-Opitz syndrome. Nat Genet 43, 729-31 (2011).
7. Gibson, W. T. et al. Mutations in EZH2 Cause Weaver Syndrome. Am J Hum Genet 90, 110-8 (2012).
8. Tatton-Brown, K. et al. Germline mutations in the oncogene EZH2 cause Weaver syndrome and increased human height. Oncotarget 2, 1127-33 (2011).
9. Campeau, P. M. et al. Mutations in KAT6B, Encoding a Histone Acetyltransferase, Cause Genitopatellar Syndrome. Am J Hum Genet (2012).
10. Clayton-Smith, J. et al. Whole-exome-sequencing identifies mutations in histone acetyltransferase gene KAT6B in individuals with the Say-Barber-Biesecker variant of Ohdo syndrome. Am J Hum Genet 89, 675-81 (2011).
11. Simpson, M. A. et al. De Novo Mutations of the Gene Encoding the Histone Acetyltransferase KAT6B Cause Genitopatellar Syndrome. Am J Hum Genet (2012).
12. van Bokhoven, H. Genetic and epigenetic networks in intellectual disabilities. Annu Rev Genet 45, 81-104 (2011).
13. Tatton-Brown, K. et al. Mutations in the DNA methyltransferase gene DNMT3A cause an overgrowth syndrome with intellectual disability. Nat Genet 46, 385-8 (2014).
14. Luscan, A. et al. Mutations in SETD2 cause a novel overgrowth condition. J Med Genet 51, 512-7 (2014).
15. Bajpai, R. et al. CHD7 cooperates with PBAF to control multipotent neural crest formation. Nature 463, 958-62 (2010).
16. Simoes-Costa, M. & Bronner, M. E. Insights into neural crest development and evolution from genomic analysis. Genome Res 23, 1069-80 (2013).
17. Micucci, J. A. et al. CHD7 and retinoic acid signaling cooperate to regulate neural stem cell and inner ear development in mouse models of CHARGE syndrome. Hum Mol Genet 23, 434-48 (2014).
18. Schulz, Y. et al. CHD7, the gene mutated in CHARGE syndrome, regulates genes involved in neural crest cell guidance. Hum Genet 133, 997-1009 (2014).
19. Sperry, E. D. et al. The chromatin remodeling protein CHD7, mutated in CHARGE syndrome, is necessary for proper craniofacial and tracheal development. Dev Dyn 243, 1055-66 (2014).
20. Hurd, E. A. et al. Loss of Chd7 function in gene-trapped reporter mice is embryonic lethal and associated with severe defects in multiple developing tissues. Mamm Genome 18, 94-104 (2007).
21. Hsu, P. et al. CHARGE syndrome: A review. J Paediatr Child Health 50, 504-11 (2014).
22. Issekutz, K. A., Graham, J. M., Jr., Prasad, C., Smith, I. M. & Blake, K. D. An epidemiological analysis of CHARGE syndrome: preliminary results from a Canadian study. Am J Med Genet A 133A, 309-17 (2005).
23. Blake, K. D. et al. CHARGE association: an update and review for the primary pediatrician. Clin Pediatr (Phila) 37, 159-73 (1998).
24. Vissers, L. E. et al. Mutations in a new member of the chromodomain gene family cause CHARGE syndrome. Nat Genet 36, 955-7 (2004).
25. Janssen, N. et al. Mutation update on the CHD7 gene involved in CHARGE syndrome. Hum Mutat 33, 1149-60 (2012).
26. Bartels, C. F., Scacheri, C., White, L., Scacheri, P. C. & Bale, S. Mutations in the CHD7 gene: the experience of a commercial laboratory. Genet Test Mol Biomarkers 14, 881-91 (2010).
27. Schnetz, M. P. et al. Genomic distribution of CHD7 on chromatin tracks H3K4 methylation patterns. Genome research 19, 590-601 (2009).
28. Schnetz, M. P. et al. CHD7 targets active gene enhancer elements to modulate ES cell-specific gene expression. PLoS Genet 6, e1001023 (2010).
29. Grafodatskaya, D. et al. Multilocus loss of DNA methylation in individuals with mutations in the histone H3 lysine 4 demethylase KDMSC. BMC Med Genomics 6, 1 (2013).
30. Verloes, A. Updated diagnostic criteria for CHARGE syndrome: a proposal. Am J Med Genet A 133A, 306-8 (2005).
31. Chen, Y. A. et al. Cross-Reactive DNA Microarray Probes Lead to False Discovery of Autosomal Sex-Associated DNA Methylation. Am J Hum Genet in press(2012).
32. Chen, Y. A. et al. Discovery of cross-reactive probes and polymorphic CpGs in the Illumina Infinium HumanMethylation450 microarray. Epigenetics 8, 203-209 (2013).
33. Ng, S. B. et al. Exome sequencing identifies MLL2 mutations as a cause of Kabuki syndrome. Nat Genet 42, 790-3 (2010).
34. Banka, S. et al. How genetically heterogeneous is Kabuki syndrome?: MLL2 testing in 116 patients, review and analyses of mutation and phenotypic spectrum. Eur J Hum Genet 20, 381-8 (2012).
35. Grafodatskaya, D. et al. Multilocus loss of DNA methylation in individuals with mutations in the histone H3 lysine 4 demethylase KDM5C. BMC Med Genomics 6, 1 (2013).
36. Bogershausen, N. & Wollnik, B. Unmasking Kabuki syndrome. Clin Genet (2012).
37. Fischbach, G. D. & Lord, C. The Simons Simplex Collection: a resource for identification of autism genetic risk factors. Neuron 68, 192-5 (2010).
38. Chen, Y. A. et al. Cross-Reactive DNA Microarray Probes Lead to False Discovery of Autosomal Sex-Associated DNA Methylation. Am J Hum Genet in press(2012).
39. Chen, Y. A. et al. Discovery of cross-reactive probes and polymorphic CpGs in the Illumina Infinium HumanMethylation450 microarray. Epigenetics 8, 203-209 (2013).

1. A method of detecting and/or screening for CHARGE syndrome (CS), or an increased likelihood of CS, in a human subject, comprising:
determining a sample methylation profile from a sample comprising DNA from said subject, said sample profile comprising the methylation level of at least 3, optionally at least 5, at least 8, at least 10, at least 25, at least 44, at least 50, at least 75, at least 100, at least 125, at least 140, or all CpG loci from (i) Tables 2 and/or 16 and/or (ii) associated CpG loci residing within 300 nucleotides, optionally within 150 nucleotides, of the CpG loci of (i); and
(a) determining the level of similarity of said sample profile to one or more control profiles, wherein (i) a high level of similarity of the sample profile to a CS specific control profile; (ii) a low level of similarity to a non-CS control profile: and/or (iii) a higher level of similarity to a CS specific control profile than to a non-CS control profile indicates the presence of, or an increased likelihood of, CS; and/or
(b) determining a sample methylation profile from a sample comprising DNA from said subject, said sample profile comprising the methylation level of at least 2, optionally at least 3, at least 4, at least 6, at least 8, at least 10, at least 16, at least 20, at least 25, at least 30, at least 35, at least 40, or all the genes from Tables 2 and/or 16: and
determining the level of similarity of said sample profile to one or more control profiles, wherein (i) a high level of similarity of the sample profile to an CS specific control profile; (ii) a low level of similarity to a non-CS control profile: and/or (iii) a higher level of similarity to a CS specific control profile than to a non-CS control profile indicates the presence of or an increased likelihood of, CS.

2. The method of claim 1, wherein the selected CpG lad comprise CpG loci from Tables 2 and/or 16 having an absolute CS delta-beta value≧0.10, ≧0.11, ≧0.12, ≧0.13, ≧0.15, ≧0.18, ≧0.20 or ≧0.22; and/or (ii) associated CpG loci residing within 300 nucleotides, optionally within 150 nucleotides, of the CpG loci of (I).

3-12. (canceled)

13. The method of claim 1, wherein a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25; and/or wherein a higher level of similarity to the CS specific profile than to the non-CS control profile is indicated by a higher correlation value computed between the sample profile and the CS specific profile than an equivalent correlation value computed between the sample profile and the non-CS control profile, optionally wherein the correlation value is a correlation coefficient.

14. (canceled)

15. (canceled)

16. The method of claim 1, wherein methylation level is measured as a β-value.

17-22. (canceled)

23. The method of claim 1, wherein the sample is derived from blood, fibroblast tissue, buccal tissue, lymphoblastoid cell line, saliva or a prenatal sample, optionally a CVS, placenta, circulating fetal DNA and/or amniotic fluid sample.

24. The method of claim 1, wherein he human subject is a fetus,

25-30. (canceled)

31. A method of determining a course of management for an individual with CHARGE syndrome (CS), or an increased likelihood of CS, comprising:
a) identifying an individual with CS or an increased likelihood of CS, according to the method of claim 1; and
b) assigning a course of management for CS and/or symptoms of a CS, comprising) testing for at least one medical condition associated with CS and ii) applying an appropriate medical intervention based on the results of the testing.

32. The method of claim 31, wherein the medical condition is selected from ophthalmic colobomas, cardiovascular anomalies, hearing loss, airway conditions such as choanal atresia/stenosis or tracheoesophageal fistula, feeding issues, retinal detachment, growth delay, delayed puberty, renal anomalies, developmental difficulties, behavioural problems, dual sensory loss and neuropsychological issues such as attention deficit hyperactivity disorder or autism.

33. A kit for detecting and/or screening for CHARGE syndrome, or an increased likelihood of CS, in a sample, comprising:
a) at least one detection agent for determining the methylation level of:
i) at least 3, optionally at least 5, at least 8, at least 10, at least 25, at least 44, at least 50, at least 75, at least 100. at least 125, at least 140, or all CpG loci from (i) Tables 2 and/or 16 and/or (ii) associated CpG loci residing within 300 nucleotides, optionally within 150 nucleotides, of the CpG loci of (1); and/or
ii) at least 2, optionally at least 3, at least 4, at least 6, at least 8, at least 10, at least 16, at least 20, at least 25, at least 30, at least 35, at least 40, or all the genes from Tables 2 and/or 16; and
b) instructions for use. 34, (Currently amended) The kit according to claim 33, further comprising bisulfite conversion reagents, methylation-dependent restriction enzymes, methylation-sensitive restriction enzymes, PCR reagents, probes, primers and/or a computer-readable medium that causes a computer to compare methylation levels from a sample at the selected CpG loci to one or more control profiles and compute a correlation value between the sample and control profile.

35. (canceled)

36. A method of detecting and/or screening for Kabuki syndrome (KS), or an increased likelihood of KS, in a human subject, comprising:
determining a sample methylation profile from a sample comprising DNA from said subject, said sample profile comprising the methylation level of at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and
(a) determining, the level of similarity of said sample profile to one or more control profiles, wherein (i) a high level of similarity of the sample profile to a KS specific control profile; (ii) a low level of similarity to a non-KS control profile; and/or (iii) a higher level of similarity to a KS specific control profile than to a non-KS control profile indicates the presence of, or an increased likelihood of, KS: and/or
(b) determining a sample methylation profile from a sample comprising DNA from said subject, said sample profile comprising the methylation level of at least 3, optionally at least 4, at least 6. at least 8 at least 10 at least 15, at least 20 at least 25, at least 50, at least 75, at least 100, at least 125, or all the genes from Tables 9 and/or 17; and
determining the level of similarity of said sample profile to one or more control profiles, wherein (i) a high level of similarity of the sample profile to an KS specific control similarity to a KS specific control profile than to non-KS control profile indicates the presence of, or an increased likelihood of, KS.

37. The method of claim 36, wherein the selected CpG loci comprise CpG loci from Tables 2 and/or 16 having an absolute KS delta-beta value ≧0.15, optionally ≧0.16, ≧0.18, ≧0.20, ≧0.22, ≧0.24 or ≧0.25; and/or (ii) associated CpG loci residing within 300 nucleotides, optionally within 150 nucleotides, of the CpG loci of (i).

38-45. (canceled)

46. The method of claim 36, wherein a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25; and/or wherein a higher level similarity to the KS specific profile than to the non-KS control profile is indicated by a higher correlation value computed between the sample profile and the KS specific profile than an equivalent correlation value computed between the sample profile and the non-KS control profile, optionally wherein the correlation value is a correlation coefficient.

47. (canceled)

48. (canceled)

49. The method of claim 36, wherein methylation level is measured as a β-value.

50-55. (canceled)

56. The method of claim 36, wherein the sample is derived from blood, fibroblast tissue, buccal tissue, lymphoblastoid cell line, saliva or a prenatal sample, optionally a CVS, placenta, circulating fetal DNA and/or amniotic fluid sample,

57. The method of claim 36, wherein the human subject is a fetus.

58-63. (canceled)

64. A method of determining a course of management for an individual with Kabuki syndrome (KS), or an increased likelihood of KS, comprising:
a) identifying an individual with KS or an increased likelihood of KS according to the method of claim 36; and
b) assigning a course of management for KS and/or symptoms of a KS, comprising i) testing for at least one medical condition associated with KS and ii) applying an appropriate medical intervention based on the results of the testing.

65. The method of claim 64 wherein the medical condition is selected from ophthalmic abnormalities, cardiovascular anomalies, hearing loss, kidney, abnormalities, skeletal anomalies, dental abnormalities, feeding difficulties, endocrine problems, infection, autoimmune disorders, seizures and developmental disorders.

66. A kit for detecting and/or screening for Kabuki syndrome, or an increased likelihood of KS, in a sample, comprising:
a) at least one detection agent for determining the methylation level of;
iii) at least 6, optionally at least 8, at least 10, at least 15, at least 20, at least 25, at least 46, at least 50, at least 75, at least 100, at least 125, at least 150, at least 200, at least 250, or all CpG loci from (i) Tables 9 and/or 17 and/or (ii) associated CpG loci residing within 300 nucleotides, optionally within 150 nucleotides, of the CpG loci of (i); and/or
iv) at least 3, optionally at least 4, at least 6, at least 8, at least 10, at least 15, at least 20, at least 25, at least 50, at least 75, at least 100, at least 125, or all the genes from Tables 9 and 17; and
b) instructions for use.

67. The kit according to claim 66, further comprising bisulfite conversion reagents, methylation-dependent restriction enzymes, methylation-sensitive restriction enzymes, PCR reagents, probes, primers and/or a computer-readable medium that causes a computer to compare methylation levels from a sample at the selected CpG loci to one or more control profiles and compute a correlation value between the sample and control profile.

68. (canceled)
